SEQUENCE LISTING (20 sequences)

SEQ ID NO: 1 - DNA, 2751 bp, Mus musculus
cctagggtctcagggctaagctgggtctcaagtggggggcagacagagaggtgatccagtgctgaaaacactgctggagccagggtgtctgggagctgctgggctagaagcaagaaggcagcagaaggaaggccagcttaaagacagcctacggtggatcatgggctctatgttccggagtgaagaggtggccttggtccagctcctgttgcccacagggtctgcttacaactgtgtgagccagcttggtgagctgggcctcgtggagttcagagacctcaacgaatccgtgagcgccttccagagacgcttcgtggtagacgtccagcgctgcgaggaactggagaagacgtttaccttcttgcgggaagaagtgcagcgggcaggtctgacgctggccccacctgaggggacactgccagcaccacctccccgagacctgctgcgcatccaggaggaaacggatcgcttggcccaggagcttcgggatgttcgtggcaatcagcaggcactgcgggcccagctgcaccagctgcggcttcactcggctgtgctgggccagagccacagcccgccggtggcagctgaccatactgaaggacccttctctgagacaacacccctgcttccaggcacccgggggccacattcagacctgaaggtcaattttgtggcaggtgctgtggagccctacaaggctgcggccctggagcgcctgctgtggagagcttgccgcggcttcctcattgccagctttcgggagactgaggggcagctggaggacccagtgacgggtgagcctgcaacttggatgacctttgtcatctcctactggggtgagcagatcggacagaagatccgaaagatcacagactgcttccactgtcatgtcttcccatacctggagcaggaggaagcccggttcaggaccttgcagcagctgcagcagcaaagccaggagcttcaggaagtcctgggggagacagaccggttcctgagccaggtgttgggccgggtgcagcagctgctgcccccatggcaggtgcagatccacaagatgaaggcagtgtacctgaccctcaaccagtgcagtgtgaacaccacacacaagtgcctcatcgcggaggtctggtgtgccgcgagggacctgcccactgtgcagcaggcgctgcagagcggctcaagtgaggaaggagtgagtgctgtcgctcaccgcatcccctgccaggacatgcctccaaccctcatcaggaccaaccgcttcacctccagcttccagggcattgtggatgcctatggtgtgggccgctacagggaagttaaccctgctccctacaccatcatcacattcccctttctcttcgctgtgatgtttggcgatgtggggcacggactgctcatgtttctctttgccctggccatggtcctcactgaaaaccgtccagctgtgaaggctgcacaaaacgagatctggcagaccttctttgggggtcgatacctactcctgctcatgggtctgttctccgtctacactggcttcatctacaatgagtgcttcagccgagccaccactattttcccctcaggttggagtgtggctgccatggccaaccagtcaggctggagtgacgagtatctgtcccagcactccatgctcaccctgaaccctaacatcactggtgtcttccttggaccatatccctttggcattgacccgatctggagcctggccacgaaccacctgagctttctcaactccttcaagatgaagatgtctgtcatccttggggtcacgcacatggcctttggggtgttcctcagcatcttcaaccacgtgcactttggccagtcccaccggctactgctggagaccttgcctgagctcatcttcctgctgggtctctttggctaccttgtgttcctcattgtctacaagtgggtgaatgtctcagctgctagcgcctcctcggcccccagtatcctcattcacttcatcaatatgttcctcttctctcaaaaccccaccaatcatctgctcttccatgggcaggaagtggtacagtacgtgctggtggtcctggctttggctaccgttcctatcctgttgctgggcacacccttgtacctgctgcgccagcaccgccacagaagaaacactcagagaaggccagcaggccagcaggatgaggacacggacaagcttctggcctcccctgatgcatccaccttggagaacagctggagccctgatgaggagaaggctgggagcccaggggatgaagaaactgagtttgtcccttctgagatcttcatgcaccaagcaatccacaccattgagttctgcctgggctgcatctccaacacagcctcctacttgcgtctctgggccctgagcctggcccatgcccagctgtctgaggtcctgtgggccatggtgatgcgcataggcctgggcatgggccgagagatcggcgtggcagctgtggtgctggtccctgtgtttgctgcctttgctgtgttgactgtggccatcctgttagtgatggaggggctctcagccttcctgcatgccctgcggttgcactgggtggagttccagaacaagttctattcaggcaccggctacaagctcagccccttcaccttcactgtggacagtgactagctacccactgcaatccctgcttctccgaagtgggacaggaataaagagctggctggcccagaaaaaaaaaaaaaaaaaaaaaaaaa

SEQ ID NO: 2 - protein, 834 aa, Mus musculus
MGSMFRSEEVALVQLLLPTGSAYNCVSQLGELGLVEFRDLNESVSAFQRRFVVDVQRCEELEKTFTFLREEVQRAGLTLAPPEGTLPAPPPRDLLRIQEETDRLAQELRDVRGNQQALRAQLHQLRLHSAVLGQSHSPPVAADHTEGPFSETTPLLPGTRGPHSDLKVNFVAGAVEPYKAAALERLLWRACRGFLIASFRETEGQLEDPVTGEPATWMTFVISYWGEQIGQKIRKITDCFHCHVFPYLEQEEARFRTLQQLQQQSQELQEVLGETDRFLSQVLGRVQQLLPPWQVQIHKMKAVYLTLNQCSVNTTHKCLIAEVWCAARDLPTVQQALQSGSSEEGVSAVAHRIPCQDMPPTLIRTNRFTSSFQGIVDAYGVGRYREVNPAPYTIITFPFLFAVMFGDVGHGLLMFLFALAMVLTENRPAVKAAQNEIWQTFFGGRYLLLLMGLFSVYTGFIYNECFSRATTIFPSGWSVAAMANQSGWSDEYLSQHSMLTLNPNITGVFLGPYPFGIDPIWSLATNHLSFLNSFKMKMSVILGVTHMAFGVFLSIFNHVHFGQSHRLLLETLPELIFLLGLFGYLVFLIVYKWVNVSAASASSAPSILIHFINMFLFSQNPTNHLLFHGQEVVQYVLVVLALATVPILLLGTPLYLLRQHRHRRNTQRRPAGQQDEDTDKLLASPDASTLENSWSPDEEKAGSPGDEETEFVPSEIFMHQAIHTIEFCLGCISNTASYLRLWALSLAHAQLSEVLWAMVMRIGLGMGREIGVAAVVLVPVFAAFAVLTVAILLVMEGLSAFLHALRLHWVEFQNKFYSGTGYKLSPFTFTVDSD

SEQ ID NO: 3 - DNA, 2742 bp, Mus musculus
gctatatgtgcgcctgcgccgcgctgctgcggggccagtcgggacggaggagacaagatggcgttgcgagcgatgcggggaatcgtgaacggggccgcacccgaactgcccgtgcccaccggtgggccgatggccggagctcgggagcaggcgctggcggtgagccggaactacctatcccagcctcgtctcacctacaagactgtctctggggtgaatggtccactagtgatcctagatcatgtgaagtttcccagatacgctgagattgtccacttgacattaccagatggcacaaagagaagtgggcaagtcctagaagttagtggctccaaagcagtggttcaggtatttgaagggacatctggtatagacgccaagaaaacatcctgtgagtttactggagatattctccgaacaccagtgtctgaggatatgcttggtcgagtattcaacggatcaggaaaacccattgaccgaggccctgtggtgctggctgaagacttccttgacatcatgggtcagccaatcaaccctcagtgtcggatctatccagaagagatgattcagacgggcatttccgccatcgatggcatgaacagtattgctaggggacagaaaatccccatcttttctgctgctggattaccacacaacgagattgcagctcagatctgtcgccaggctggtttggtaaagaaatccaaagatgtagtggactatagtgaagaaaattttgccattgtgtttgctgctatgggagtaaacatggaaacagcccggttctttaaatctgactttgaagaaaatggctcaatggacaatgtctgcctttttttgaatctggctaatgacccaaccattgagcggatcatcactcctcgcctggctctgaccacggccgagtttctggcatatcagtgtgagaagcacgtgctggtgatcctgacggacatgagctcctacgctgaagcgctgcgagaggtttcagctgccagggaagaggtccctggtcggcgaggcttcccaggctacatgtataccgacttagccacaatctatgaacgtgccggtcgagtggaaggtagaaacggctctattacccaaatccctattctcaccatgcccaatgacgatatcactcatcccatccctgacttgactgggtacattactgagggccagatctatgtggacagacagctgcacaacagacagatttaccctcctattaatgtgctgccctcactctctcggttaatgaagtcagctatcggagaaggaatgaccagaaaggatcacgctgatgtgtctaaccagttgtatgcgtgctatgccatcggtaaggacgtgcaagccatgaaagccgtggtgggagaggaagccctgacctcagatgatctcctttacctggaatttctgcagaagtttgagaagaacttcattactcagggtccctatgaaaaccgaactgtttatgagactttggacattggctggcagttgcttcgaatcttccccaaagaaatgctgaagagaatccctcagagcaccctgagcgaattttaccctcgagactctgcaaaacactagtagctgctgctgcttgtgtggcgtgaccctcttgtgaagtactggttctgtttcctgattccttttgcactcctccatcccacctgtgagtgggagtttacctgttaccctgtaattaaaaacaaaggctaggtaactattgtgccagtgttgacagtttaaactgctaacagattgagagatccctgctcagaaacctcacctttagtgctttctttaaagaagctgaggggtgaagtttgcttactgatgtatctatttgtacagatagtggagagctagttgctaataatgtcttgtttgggtctcccaaaccctacctctcaactcccttaagagtatcactgttctgaagctaaaatgcttcagtctcaaatttaggggcaaagtggagactggaagaattctcctttcagaagaaccacgaggctctggctgagcctccctctggagtgctagtgtacctgtgggtctgtcctctgctctgtgcagatgggttttactgtctgctgtgttttcttacgaaaagagtttcgttctgccagtgctgtgagttgggaattcctgcgtggcgactttctctgaggcccacagagaatcaggtaacattgcacagcctgcgttctcctgcagtggtggcacgccttgtgctgacgacccccactgggctacagtcacaggagaattgagacttggaaaaggcttgggtacaattaagaaaaaccctacatcccaccctcctcttgacgcgtttctggtggtttcggtttctgcgttgccctccagatcctgaggtgggccgtgagacgacttgctttaaactctgttcaatccccaggctttctgagtccgctctaccttttctgggtgatcttgtggcacatgtagcaatgtttctttcattcctgtcccttcctggcttgagctcttggctgtattctgtgtgcctctaccatgtctgctgtttgggtctctgcgctgtgtgttctcaggtgcagccatgactccccactctgtgcattccaccttccagttgtttttctctgagggggtagtgggggggtcagcatgattatattttaatgtagaaaatgtgacatctcgttataaatgaaaataaatgttaaattaaatggaagttacctgaaa

SEQ ID NO: 4 - protein, 511 aa, Mus musculus
MALRAMRGIVNGAAPELPVPTGGPMAGAREQALAVSRNYLSQPRLTYKTVSGVNGPLVILDHVKFPRYAEIVHLTLPDGTKRSGQVLEVSGSKAVVQVFEGTSGIDAKKTSCEFTGDILRTPVSEDMLGRVFNGSGKPIDRGPVVLAEDFLDIMGQPINPQCRIYPEEMIQTGISAIDGMNSIARGQKIPIFSAAGLPHNEIAAQICRQAGLVKKSKDVVDYSEENFAIVFAAMGVNMETARFFKSDFEENGSMDNVCLFLNLANDPTIERIITPRLALTTAEFLAYQCEKHVLVILTDMSSYAEALREVSAAREEVPGRRGFPGYMYTDLATIYERAGRVEGRNGSITQIPILTMPNDDITHPIPDLTGYITEGQIYVDRQLHNRQIYPPINVLPSLSRLMKSAIGEGMTRKDHADVSNQLYACYAIGKDVQAMKAVVGEEALTSDDLLYLEFLQKFEKNFITQGPYENRTVYETLDIGWQLLRIFPKEMLKRIPQSTLSEFYPRDSAKH

SEQ ID NO: 5 - DNA, 34 bp, Mus musculus
gaattcggtggtatgggggagcttttccggagtg

SEQ ID NO: 6 - DNA, 40 bp, Mus musculus
gtcgactagatgacagtgtacggagctgggttaatctctc

SEQ ID NO: 7 - DNA, 33 bp, Mus musculus
gaattcggtggtatgggctctctcttccgcagc

SEQ ID NO: 8 - DNA, 45 bp, Mus musculus
gtcgactagatgatggtaaagagagctgggttcacttctctgtag

SEQ ID NO: 9 - DNA, 39 bp, Mus musculus
gaattcggtggtatgggctctatgttccggagtgaagag

SEQ ID NO: 10 - DNA, 36 bp, Mus musculus
gtcgacattaggtgtagggagcagggttaacttccc

SEQ ID NO: 11 - DNA, 42 bp, Mus musculus
gaattcggtggtatggcatctgtgtttcgaagtgaggagatg

SEQ ID NO: 12 - DNA, 41 bp, Mus musculus
gtcgactagatgatagtgtagggagctgggtttatctctcg

SEQ ID NO: 13 - DNA, 30 bp, Mus musculus
gaattcggtggtatggccacaacagtagac

SEQ ID NO: 14 - DNA, 60 bp, Mus musculus
gtcgacttaaccaccgtggtgatggtgatgatgaccgccgagcgccgtgtcggatgcggg

SEQ ID NO: 15 - DNA, 25 bp, Mus musculus
gaattcggtggtatggcgttgcgag

SEQ ID NO: 16 - DNA, 69 bp, Mus musculus
gtcgacttaaccaccgtggtgatggtgatgatgaccgccgtgttttgcagagtctcgagggtaaaattc

SEQ ID NO: 17 - DNA, 25 bp, Mus musculus
gaattcggtggtatggcgttgcgag

SEQ ID NO: 18 - DNA, 40 bp, Mus musculus
gtcgactaacaggatgttttcttggcgtctataccagatg

SEQ ID NO: 19 - DNA, 39 bp, Mus musculus
gaattcggaggagaagacttccttgacatcatgggtcag

SEQ ID NO: 20 - DNA, 28 bp, Mus musculus
gtcgacctagtgttttgcagagtctcga

(19) United States
(12) Patent Application Publication
(10) Pub. No.: US 2011/0189306 A1
(43) Pub. Date: Aug. 4, 2011

(54) COMPOUNDS, COMPOSITIONS AND TREATMENTS FOR V-ATPase RELATED DISEASES

(76) Inventors: Norbert Kartner, Toronto (CA); Morris Frank Manolson, Toronto (CA)

(21) Appl. No.: 13/005,921

(22) Filed: Jan. 13, 2011

Related U.S. Application Data
(60) Provisional application No. 61/294,809, filed on Jan. 13, 2010.

(51) Int. Cl. A61K 33/16; C07C 243/38; A61K 31/166; C07D 317/46; A61K 31/36; A61K 38/22; A61K 38/23; A61K 31/56; A61K 31/59; A61P 19/00; A61P 35/00

(52) U.S. Cl. 424/676; 564/150; 514/615; 549/438; 514/466; 514/9.7; 514/11.9; 514/171; 514/167

(57) ABSTRACT

The present invention relates to therapeutic and/or prophylactic uses of hydrazide compounds and to pharmaceutical compositions containing one or more of these compounds as an active component for treating a disease or disorder requiring modulation of vacuolar (H+)-ATPases.

### CROSS-REFERENCE TO RELATED APPLICATION

This application claims the benefit of U.S. Provisional Application No. 61/294,809, filed Jan. 13, 2010, the entire disclosure of which is incorporated herein by reference.

### FIELD OF INVENTION

The invention relates to compositions and methods for treatment of disorders or diseases requiring modulation of vacuolar (or vacuolar-type or V-type) (H+)-ATPases (V-ATPases).

### BACKGROUND OF INVENTION

Bone loss is seen in many diseases, including osteoporosis (OP) and inflammatory arthritis (IA). OP alone accounts for 11.4 million sufferers in North America, with 38 million at risk. Annually, 2.2 million OP-related fractures incur direct costs of treatment exceeding $20 billion. Of these, approximately 300,000 are OP-related hip surgery patients, with nearly 20% dying within a year of surgery and half of the survivors remaining disabled and suffering markedly reduced quality of life, and lifespan, with added indirect costs. Present treatments have limited efficacy and potentially serious side effects. Development of therapeutics with high specificity and efficacy to restore and maintain normal bone mineral density is highly desirable.

Osteoclasts (OCs) are the cells that demineralize bone matrix and V-ATPases are the multi-subunit molecular pumps in the OC plasma membrane (PM) that are responsible for the required acid secretion.(see FIG. 1) Increased osteoclast activity can lead to excessive bone loss. V-ATPases present on the osteoclast membrane pump acid onto the bone surface to dissolve bone, making them potential drug targets. It would be of value to be able to selectively modulate the V-ATPases or acid pump.

### SUMMARY OF INVENTION

The present invention relates to therapeutic and/or prophylactic uses of hydrazide compounds and to pharmaceutical compositions containing one or more of these compounds as an active component modulating V-ATPases, in particular V-ATPase-mediated secretion by mammalian cells.

Accordingly, the invention provides a hydrazide compound and compositions comprising a hydrazide compound. Hydrazide compounds and compositions can be in therapeutically effective amounts for modulating V-ATPases in a subject. In embodiments, the invention provides a hydrazide compound and compositions comprising a hydrazide compound in a therapeutically effective amount for controlling V-ATPase-mediated acid secretion in a subject. In embodiments, the invention provides a hydrazide or a composition comprising a hydrazide compound in a therapeutically effective amount for treating a disease or disorder disclosed herein, in particular a disease or disorder involving disruptions to normal V-ATPase activity and/or function or requiring modulation of V-ATPase-mediated secretion. In embodiments, the invention provides a hydrazide or a composition comprising a hydrazide compound in a therapeutically effective amount for treating a disease or disorder, or requiring regulation of biological phenomena including, but not limited to: intra-organellar acidification of intracellular organelles; urinary acidification; bone resorption; inflammatory arthritis, osteoarthritis, periodontal disease, fertility; angiogenesis; cellular invasiveness (e.g., tumor cell invasiveness); tumor cell proliferation and metastasis; and the development of drug resistance in tumor cells. In particular embodiments the invention provides a hydrazide or a composition comprising a hydrazide compound in a therapeutically effective amount for treating a disease associated with loss of bone mass in a subject. The compositions of the invention generally comprise a hydrazide compound in a pharmaceutically acceptable carrier, excipient, or vehicle.

In aspects, a pharmaceutical composition of the invention comprises a therapeutically effective amount of a hydrazide compound to provide a beneficial effect, in particular a sustained beneficial effect following treatment.

The invention further provides methods for preparing a composition of the invention. In an aspect, the invention provides a method of preparing a pharmaceutical composition comprising a hydrazide compound adapted for use in a disease or disorder disclosed herein. A method can comprise mixing one or more hydrazide compound and optionally a pharmaceutically acceptable carrier, excipient, or vehicle. A pharmaceutically acceptable carrier, excipient, or vehicle may be selected that is effective to physically stabilize the hydrazide compound(s). After compositions have been prepared, they can be placed in an appropriate container and labeled for treatment of an indicated condition. For administration of a composition of the invention, such labeling would include amount, frequency, and method of administration.

In some aspects, the invention provides methods to make commercially available pills, tablets, caplets, soft and hard gelatin capsules, lozenges, sachets, cachets, vegicaps, liquid drops, elixirs, suspensions, emulsions, solutions, syrups, aerosols (as a solid or in a liquid medium) suppositories, sterile injectable solutions, and/or sterile packaged powders, which contain a hydrazide compound adapted for use in a disease or disorder disclosed herein.

The invention also contemplates the use of one or more hydrazide compound, composition or method of the invention to prevent and/or ameliorate severity, symptoms, and/or reduce periodicity of recurrence of a disease or disorder disclosed herein. Therefore, the invention contemplates the prevention and/or treatment, in a subject, of a disease or disorder disclosed herein, using a hydrazide compound or a composition of the invention. In particular, the invention provides a method for treating a disease or disorder disclosed herein in a subject comprising administering to the subject a therapeutically effective amount of one or more hydrazide compound or a composition of the invention. A method of the invention can be used therapeutically or prophylactically in a subject susceptible to or having a predisposition to a disease or disorder disclosed herein.

In an aspect, the invention provides a method for the prevention and/or intervention of a disease or disorder disclosed herein in a subject comprising administration of at least one hydrazide compound or composition of the invention to the subject.

The invention provides a method of treating a disease or disorder disclosed herein comprising administering at least one hydrazide compound or a composition of the invention to a subject in need thereof to thereby produce beneficial effects, in particular sustained beneficial effects following treatment. In an embodiment, the compound or composition is administered orally or systemically.

In an aspect, the invention provides a method for ameliorating progression of a disease or disorder disclosed herein, or obtaining a less severe stage of a disease or disorder disclosed herein in a subject suffering from such disease or disorder comprising administering a therapeutically effective amount of one or more hydrazide compound or a composition of the invention.

The invention relates to a method of delaying the progression of a disease or disorder disclosed herein comprising administering a therapeutically effective amount of one or more hydrazide compound or a composition of the invention.

The invention also relates to a method of increasing survival of a subject suffering from a disease or disorder disclosed herein comprising administering a therapeutically effective amount of one or more hydrazide compound or a composition of the invention.

In an embodiment, the invention relates to a method of improving the lifespan of a subject suffering from a disease or disorder disclosed herein comprising administering a therapeutically effective amount of one or more hydrazide compound or a composition of the invention.

The compositions and methods are useful in the prevention, palliation, and/or treatment of a disease or disorder disclosed herein and their manifestations irrespective of the origin of the condition in a subject. In particular, the compositions and methods of the invention are useful for the treatment and/or prophylaxis of diseases associated with loss of bone mass, such as osteoporosis and related osteopenic diseases, Paget's disease, hyperparathyroidism and related diseases. The compositions and methods of the invention are also considered to possess anti-tumour activity, antiviral activity (for example against Semliki Forest, Vesicular Stomatitis, Newcastle Disease, Influenza A and B, HIV viruses), antiulcer activity (e.g., for treating chronic gastritis and peptic ulcer induced by Helicobacter pylori), immunosuppressant activity, antilipidemic activity, anti-atherosclerotic activity and to be useful for the treatment of AIDS and neurodegenerative diseases such as Alzheimer's disease. The compositions and methods of the invention are also considered useful in inhibiting angiogenesis and may be useful in treating angiogenic diseases such as rheumatoid arthritis, diabetic retinopathy, psoriasis and solid tumours.

The hydrazide compounds and compositions are useful for treating subjects afflicted with pathological bone loss, including but not restricted to conditions such as osteoporosis, inflammatory arthritis, periodontal disease and metastatic bone cancer. In an embodiment, the hydrazide compounds and compositions are used to inhibit V-ATPase-mediated acid secretion in mammalian osteoclasts, thereby reducing bone loss. Hydrazide compounds and compositions may also be used to limit or prevent cancer metastasis. In an embodiment the hydrazide compounds and compositions are used to inhibit V-ATPase-mediated acid secretion in mammalian tumour cells, thereby reducing cancer invasion and metastasis.

In an embodiment, the present invention provides a method for increasing bone density in a mammal in need thereof comprising administering to said mammal an effective amount of a hydrazide compound of the invention.

In embodiments, the invention also contemplates a method for enhancing bone formation in a mammal in need thereof by administering to the mammal an effective amount of a hydrazide compound of the invention and at least one bone enhancing agent. Examples of suitable bone enhancing agents include a synthetic hormone, a natural hormone, oestrogen, calcitonin, tamoxifen, a bisphosphonate, a bisphosphonate analog, vitamin D, a vitamin D analog, a mineral supplement, a statin drug, a selective oestrogen receptor modulator and sodium fluoride.

A treatment method of the invention may be sustained over several days, weeks, months or years thereby having a major beneficial impact on the severity of a disease or disorder and its complications.

The invention also contemplates the use of one or more hydrazide compound as a medicament, or for the preparation of a medicament for preventing and/or treating a disease or disorder disclosed herein. The invention additionally provides uses of a pharmaceutical composition of the invention as a medicament or in the preparation of medicaments for the prevention and/or treatment of a disease or disorder disclosed herein. In aspects of the invention, the medicaments provide beneficial effects, preferably sustained beneficial effects following treatment. A medicament may be in a form suitable for consumption by a subject, for example, a pill, tablet, caplet, soft and hard gelatin capsule, lozenge, sachet, cachet, vegicap, liquid drop, elixir, suspension, emulsion, solution, syrup, aerosol (as a solid or in a liquid medium) suppository, sterile injectable solution, and/or sterile packaged powder.

A composition or method of the invention may be administered to a healthy subject or a subject suffering from a disease or disorder disclosed herein. Accordingly, in an embodiment, a hydrazide compound or a composition of the invention is to be administered before or after the onset of symptoms in a subject.

The invention also provides a kit comprising a hydrazide compound or a pharmaceutical composition of the invention in kit form. In an aspect, the invention provides a kit comprising one or more hydrazide compound or composition of the invention, a container, and instructions for use in treating and/or preventing a disease or disorder disclosed herein.

These and other aspects, features, and advantages of the present invention should be apparent to those skilled in the art from the figures and following description.

### DESCRIPTION OF THE FIGURES

FIG. 1 shows a possible scenario for NTa interaction with the B subunit in the intact V-ATPase complex. For clarity, some subunits of the complex are semi-transparent. Mammalian V-ATPases have a bipartite structure consisting of an integral membrane V0 sector (subunits a, c, c″, d, e, and Ac45, with stoichiometry ac.sub.˜5 c″deAc45) and a peripherally bound V1 sector (subunits A-H, with stoichiometry A.sub.3 B.sub.3 E.sub.3 G.sub.3 DFCH) (Zhang et al., 2008). From the hollow core of the A.sub.3 B.sub.3 catalytic head group protrudes a central rotor, composed of subunits D and F. Subunits C, H and the three EG subunit heterodimers form a stator ‘cage’ that is anchored to the membrane via interaction with the N-terminal domain of subunit ‘a’, NTa. The stator allows the rotor to develop torque, upon ATP hydrolysis, to drive the proteolipid c-barrel. Rotation of the c-barrel past the interface with the C-terminal domain of the ‘a’ subunit, CTa, drives proton translocation from the cytoplasm to the lumen. Subunit e and Ac45 have unknown functions. We have shown that NTa binds to A, B and H subunits in the V1 sector. Protein structure algorithms suggest that NTa is bifurcated (Zhang et al., 2008), so it predicted here that one finger binds an AB groove in the catalytic head group, the other cradling the H subunit and possibly interacting with the d subunit (Thaker et al., 2009). The second finger to also inserts into a second, adjacent AB groove. Note that the B subunit is oriented with the N-terminal domain (NT) to the top (distal to the V1/V0 interface) and C-terminal domain (CT) to the bottom. An asterisk indicates the F-actin binding site, which is localized to the NT domain in all B subunits (Lee, et al. 1999).

FIG. 2A shows the cell population used to construct an osteoclast cDNA library for use in yeast two hybrid analysis. The photomicrograph shows TRAP staining of RANKL-differentiated RAW 264.7 osteoclasts. RAW 264.7 is a mouse tissue culture model of bone-resorbing cells, after the cultured cells have been treated with the cytokine, RANKL. Positive TRAP staining (red coloration) indicates that ostoclast differentiation has taken place. Another indication of differentiation is cell-cell fusion, producing large multi-nucleate osteoclasts. Larger osteoclasts, i.e. with more nuclei, are generally more active in bone resorption than small osteoclasts. A representative distribution of small, intermediate and large osteoclasts in the cell culture used to construct the HybriZAP-2.1 yeast two hybrid cDNA library is shown (see Materials and Methods). Cells were grown for 3 days in DMEM with 10% FBS and 100 ng/ml soluble recombinant RANKL prior to RNA extraction. Scale bar, lower right, is 100 .mu.m.

FIG. 2B shows the initial evidence that the NTa3 domain interacts with the B2 subunit. Upper panel, YRG-2 yeast cotransformed with empty pAD vector and pBD-NTa3 (.phi.•a3, top row), or pAD-B2 and pBD-NTa3 (B2•a3, bottom row) were spotted onto SD agar in two-fold serial dilutions starting at 10.sup.4 cells (left to right). On this solid histidine-containing medium, all cells are expected to grow the same, with sparser colonies as the cells are diluted more from left to right. Lower panel, same as upper panel, but on SD medium lacking histidine. In this case only cells that can activate the HIS3 gene to produce their own histidine are able to grow. All ‘a’ subunit constructs in pAD showed autoactivation (top row), so some growth was seen with expression of the NTa3 fusion alone; nevertheless, cells simultaneously expressing NTa3 and B2 fusions had a growth advantage due to activation of the HIS3 reporter gene. This is seen as growth in the right-most colonies of the bottom row. This would only occur if there is an interaction between NTa3 and B2 which enhances the transcription of the HIS3 reporter gene.

FIG. 2C shows independent verification of NTa3 and B2 interaction, using the second reporter gene of the HybriZAP yeast two hybrid system, lacZ, and affinity bead pulldown assays. Upper panel, blue staining with X-gal indicates activation of lacZ (B2•a3, upper left panel). This would only occur if there is an interaction between NTa3 and B2 which enhances the transcription of the lacZ reporter gene. Only background level staining is seen with empty vector pAD and pBD-NTa3 cotransformation (.phi.•a3, upper right panel), which is a negative control. Lower panel, pulldown assays using B2 and NTa3 as GST and TRX fusion proteins, respectively. Glutathione Sepharose 4B beads coated with GST-B2 from whole bacterial lysates were washed and exposed to lysates of bacteria expressing TRX-NTa3 (see Materials and Methods). Beads were washed, eluted and subjected to SDS-PAGE and Western blotting, using anti-His-Tag antibody (TRX contains a His-Tag). Staining with anti-GST antibody revealed the TRX-NTa3 band for pulldowns using beads coated with GST-B2 (B2, bottom left lane), but not with control empty-vector GST (GST, bottom right lane). The band in the lower left lane indicates that immobilized B2 was able to bind and pull down NTa3. The empty lane to the right indicates that TRX-NTa3 does not interact with the GST fusion partner of B2 alone.

FIG. 3A shows gel electrophoresis bands of affinity purified NTa and B subunit proteins expressed in bacteria. Affinity purified fusion proteins (2 μg, ea.) were separated on 8% SDS-PAGE and stained with Coomassie Blue 8250 (see Materials and Methods). Left panel shows proteins expressed from pET32a(+) vector; M, mol. wt. standards; followed by (2 μg each) a1, TRX-NTa1 fusion protein; a2, TRX-NTa2; a3, TRX-NTa3; a4, TRX-NTa4. Right panel shows proteins expressed from pGEX-4T-1 vector; B1, GST-B1(GH) (left lane, affinity purified on GST beads; right lane, repurified on Ni(II) beads); B2, GST-B2(GH) (left lane, affinity purified on GST beads; right lane, repurified on Ni(II) beads). Photographs are of dried gels. Major protein bands in all cases indicate high purity of the purified proteins.

FIG. 3B shows pulldowns of TRX fusions of all N-terminal ‘a’ subunit domains with GST fusions of either B1 or B2 subunits. Glutathione beads were coated with bacterial lysates from cells expressing either GST-B1 or GST-B2 fusion proteins (1 μg/ml total protein). After washing, the beads were exposed to lysates containing TRX-NTa fusions derived from a1, a2, a3 and a4 subunits (1 μg/ml total protein). Washed beads were eluted and run on 8% SDS-PAGE, blotted, and probed with anti-GST antibody. Image was developed using chemiluminescence. Note that a3 shows the highest apparent binding in the pulldown assay, especially when pulled down with B2, suggesting a uniquely high affinity for the a3-B2 interaction.

FIG. 4 shows ELISA saturation curves of B1 and B2 binding to NTa3. ELISA plates were coated with TRX-NTa3 and probed with two-fold serial dilutions of analyte (GST-B1, or GST-B2 fusion proteins, 9.8 μM to 160 nM). Binding of GST alone was negligible, as were the signals obtained without either the ligand (TRX-NTa3 fusion protein), or either analyte (data not shown). Absorbance at 450 nm was determined after staining with an anti-GST-HRP sandwich and color development with TMB (see Materials and Methods). Absorbance at the reference wavelength of 600 nm was subtracted to normalize optical variance among wells (less than 1% of maximum signal). Each curve shows means and SD bars (n=6).

FIG. 5A shows purified proteins used in pulldown assays and ELISA to localize the a3-B2 interaction within domains of B2. Left panel shows a Coomassie Blue stained gel with purified proteins run in separate lanes, as indicated. These proteins are judged to be of sufficient purity to be of use in the pulldown assays and ELISA analysis described herein. Lanes from left to right are identified as: MW, mol. wt. standards (values shown to the left); NTa3, TRX-NTa3 fusion protein (N-terminal domain of a3); B2, full length B2 fusion protein, GST-B2(GH); NTB2, GST-NTB2 (N-terminal domain of B2); CTB2, GST-CTB2 (C-terminal domain of B2). Loading was 2 μg per lane. Right panel shows a Western blot with pulldowns of TRX-NTa3 with GST-B2(GH), GST-NTB2, and GST-CTB2, as indicated. Blot was probed with anti-His-Tag antibody (TRX contains a His-Tag epitope). Note that the NTB2 pulldown is not greatly different from the control obtained with GST alone in these experiments (identical blot and exposure, with intervening lanes removed). The pulldown assay is described in Materials and Methods.

FIG. 5B shows an example of determining the domain of interaction of the B subunit with NTa3. This ELISA method is as described in FIG. 3, but compares the binding of B2 (full length B2 subunit, GST-B2(GH)) with that of CTB2 (C-terminal domain of B2, GST-CTB2) and NTB2 (the N-terminal domain of B2, GST-NTB2) to NTa3 (the N-terminal domain of a3, TRX-NTa3). Fusion protein analytes were two-fold serially diluted from 400 nM to 24.4 μM. Each curve shows means and SD bars (n=6). This figure shows that the binding site for the interaction between NTa3 and B2 is within the CTB2 domain.

FIG. 6 shows a typical primary screening result from the high-throughput ELISA. 10,000 synthetic chemical compounds from the Chembridge DIVERSet collection were screened using the ELISA method described herein, executed on a robotics platform (see Materials and Methods). Shown are results for 500 compounds where ELISA absorbances are analysed using the B-score statistic to smooth systematic machine error and take into account positive and negative controls (Brideau et al. 2003). The blue line indicates the mean and yellow lines indicate +/−3SD. Outliers with a B-score less than −10 are circled in red and are regarded as strong hits. Strong hits are retested in the primary screen and if again are deemed to be strong hits are picked for secondary screening. One such compound that is a subject of this invention is shown in FIG. 6B.

FIG. 6B shows the chemical structure of compound KM91104 (STR00001), 3,4-dihydroxy-N′-(2-hydroxybenzylidene)benzohydrazide. This compound is a subject of this invention and was picked as described in FIG. 6A, and is described further in subsequent figures as a lead candidate for development of a therapeutic for treatment of bone loss disease and cancer metastasis.

FIG. 7 shows a protein assay on cell cultures treated with various amounts of compound KM91104 to determine its effect on cellular growth. Concentration range of compound tested was from 0.3 to 40 μM, as indicated (n=3, error bars are +/−SD). Concentration of KM91104 up to 20 μM did not significantly affect cell growth. C indicates control, vehicle only added. The test cell line was undifferentiated RAW 264.7 cells.

FIG. 8 shows an MTT assay to determine cyotoxicity of compound KM91104. The concentration range of compound tested was from 0.3 to 40 μM, as indicated (n=3). Yellow MTT is converted to purple formazan by mitochondrial reductases. Formazan is measured spectrophotometrically to deduce metabolic activity of test cells. Reduced metabolic activity indicates metabolic dysfunction due to cytotoxicity. In this case, significant cytotoxicity is not seen up to 2.5 μM KM91104 concentration. C indicates control, vehicle only added. The test cell line was undifferentiated RAW 264.7 cells.

FIG. 9 shows a total solubilized TRAP assay to determine the effect of compound KM91104 on RANKL-mediated differentiation of RAW 264.7 cells. The concentration range of compound tested was from 0.3 to 40 μM, as indicated (see Materials and Methods). C indicates control, vehicle only added. TRAP is a marker for osteoclast differentiation and will convert colorless p-nitrophenylphosphate to yellow nitrophenol under acidic conditions. The yellow color is quantified spectrophotometrically and indicates degree of differentiation after 3 days of growth with RANKL. This figure shows that Differentiation is not appreciably affected up to 20 μM concentration of KM91104.

FIG. 10 shows a qualitative TRAP staining analysis and photomicrography to determine the effect of compound KM91104 on RANKL-mediated differentiation of RAW 264.7 cells. The concentration range of compound tested was from 0.6 to 40 μM, as follows (in μM): a, control, vehicle only; b, 0.63; c, 1.25; d, 2.5; e, 5.0; f, 10; g, 20; h, 40. This figure indicates that essentially all cells stain for TRAP at all concentrations of KM91104; however, there is a noticeable reduction in osteoclast size with increased compound concentration above 1.25 μM and a reduction of cell density at the highest concentration. The latter observation is consistent with the conclusion of the protein assay of FIG. 7. The observation of reduced osteoclast size in spite of consistent TRAP staining suggests that at concentrations above 1.25 μM differentiation is unaffected, but fusion of differentiated pre-osteoclasts to produce larger multinucleate cells is impaired.

FIG. 11 shows a quantitative assessment of the TRAP staining analysis, based on the methods of FIG. 10. Plotted are cell counts for multinucleated cells with greater than 3 nuclei after treatment with compound KM91104. This figure confirms the qualitative conclusion of FIG. 10 that there is a drop off in the number of larger osteoclasts above 1.25 μM concentration.

FIG. 12 shows qualitative TRAP staining analysis using osteoclasts that were RANKL-differentiated from mouse bone marrow-derived macrophages (BMM) after treatment with compound KM91104 at 1.25 μM. Panels show: a, control, vehicle only; b and c are duplicates with 1.25 μM KM91104. This figure shows that at this concentration the compound does not significantly affect the formation of large osteoclasts that are authentic osteoclasts derived from primary animal tissue culture.

FIG. 13 shows hydroxyapatite resorption by RANKL-differentiated RAW 264.7 cells in the presence of various concentrations of compound KM91104. RAW 264.7 cells (American Type Culture Collection (ATCC), cat. no. TIB-71) were differentiated in the presence of 100 ng/ml RANKL on synthetic mineralized surfaces of Corning Osteo-Assay Surface 96-well plates (Corning Inc. Life Sciences, Lowell, Mass.; cat. no. 3988XX1). Cells were cultured for 5 days in 200 μl α-MEM (Grand Island Biological Co. (GIBCO)/Invitrogen Corp. cat. no. 12571-063) with 10% fetal bovine serum (FBS; (GIBCO/Invitrogen) cat. no. 10437-028, Lot 1233072). Exposure of cells to RANKL and KM91104 was continuous throughout the experiment. The complete medium was changed on the third day. On day 5, cells were removed using 3% sodium hypochlorite solution. Plates were air dried and then imaged using digital brightfield photomicrography (Leica DM IRE2 with OpenLab software, Leica Microsystems, Richmond Hill, Canada). Image analysis was carried out with NIH ImageJ software (National Institutes of Health, Bethesda, Md.). Concentrations of KM91104 (μM) in medium were as follows: a, control, vehicle only; b, 0.63; c, 1.25; d, 2.5; e, 5.0; f, 10; g, 20; h, 40.

FIG. 14 shows the results of quantitative image analysis of resorption areas (white) shown in FIG. 13. The concentration range of KM91104 tested was from 0.3 to 40 μM, as indicated (see Materials and Methods). C indicates control, vehicle only added. Highly significant reduction in resorption was observed even at the lowest concentration. Error bars indicate +/−SD (n=3). Approximately half-maximal inhibition of resorption can be achieved with a concentration of KM91104 of 1.25 μM, which does not affect cell growth or cause cytotoxicity and which does not affect osteoclast differentiation or fusion to form large osteoclasts.

### DETAILED DESCRIPTION OF EMBODIMENTS OF THE INVENTION

For convenience, certain terms employed in the specification, examples, and appended claims are collected here.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a,” “an,” and “the” include plural referents unless the content clearly dictates otherwise. Thus, for example, reference to a composition comprising “a compound” includes a mixture of two or more compounds.

As used herein the terms “administering” and “administration” refer to a process by which a therapeutically effective amount of a compound or composition contemplated herein is delivered to a subject for prevention and/or treatment purposes. Compositions are administered in accordance with good medical practices taking into account the subject's clinical condition, the site and method of administration, dosage, patient age, sex, body weight, and other factors known to physicians.

The term “treating” refers to reversing, alleviating, or inhibiting the progress of a disease or disorder, or one or more symptoms of such disease or disorder, to which such term applies. Depending on the condition of the subject, the term also refers to preventing a disease or disorder, and includes preventing the onset of a disease or disorder, or preventing the symptoms associated with a disease or disorder. A treatment may be either performed in an acute or chronic way. The term also refers to reducing the severity of a disease or disorder or symptoms associated with such disease or disorder prior to affliction with the disorder. Such prevention or reduction of the severity of a disease or disorder prior to affliction refers to administration of a compound or composition of the present invention to a subject that is not at the time of administration afflicted with the disease or disorder. “Preventing” also refers to preventing the recurrence of a disease or disorder or of one or more symptoms associated with such disease or disorder. “Treatment” and “therapeutically,” refer to the act of treating, as “treating” is defined above. The purpose of prevention and intervention is to combat the disease or disorder and includes the administration of an active compound to prevent or delay the onset of the symptoms or complications, or alleviating the symptoms or complications, or eliminating the disease or disorder.

In aspects of the invention where the disease or disorder is osteopetrosis or osteoporosis treatment may comprise (i) partial or complete reversal of osteopetrosis or osteoporosis, (ii) prevention of, or decrease or slowing of the rate of osteopetrosis or osteoporosis, (iii) inhibition or slowing of the rate of biochemical processes which take place during osteopetrosis or osteoporosis; and/or (iv) prevention, slowing, halting and/or reversing the process of osteopetrosis or osteoporosis.

The terms “subject”, “individual”, or “patient” are used interchangeably herein and refer to an animal preferably a warm-blooded animal such as a mammal. Mammal includes without limitation any members of the Mammalia. A mammal, as a subject or patient in the present disclosure, can be from the family of Primates, Carnivora, Proboscidea, Perissodactyla, Artiodactyla, Rodentia, and Lagomorpha. In a particular embodiment, the mammal is a human. In other embodiments, animals can be treated; the animals can be vertebrates, including both birds and mammals. In aspects of the invention, the terms include domestic animals bred for food or as pets, including equines, bovines, sheep, poultry, fish, porcines, canines, felines, and zoo animals, goats, apes (e.g. gorilla or chimpanzee), and rodents such as rats and mice.

In aspects of the invention, the terms refer to organisms to be treated by the methods of the present invention. In the context of particular aspects of the invention, the term “subject” generally refers to an individual who will receive or who has received treatment (e.g., administration of a hydrazide compound(s) or compositions) for a disorder disclosed herein.

Typical subjects for treatment include persons afflicted with or suspected of having or being pre-disposed to a disease or disorder disclosed herein, or persons susceptible to, suffering from or that have suffered a disease or disorder disclosed herein. A subject may or may not have a genetic predisposition for a disease or disorder disclosed herein, such as osteopetrosis or osteoporosis. In certain aspects, a subject may be a healthy subject.

As utilized herein, the term “healthy subject” means a subject, in particular a mammal, having no diagnosed disease or disorder, infirmity, or ailment disclosed herein.

As used herein, the term “co-administered” refers to the administration of one or more hydrazide compound and additional therapeutic agent or therapies to a subject. In aspects, the administration of two or more agents/therapies is concurrent. In other aspects, a first agent/therapy is administered prior to a second agent/therapy. In this aspect, each component may be administered separately, but sufficiently close in time to provide the desired effect, in particular a beneficial, additive, or synergistic effect. The formulations, routes of administration and the appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

A “beneficial effect” refers to an effect of a hydrazide compound, a composition or treatment of the invention, including favorable pharmacological and/or therapeutic effects, and improved biological activity. In aspects of the invention, the beneficial effects include without limitation enhanced stability, a longer half life, and/or enhanced uptake. In other aspects the beneficial effects include one or more of the following: (a) reduction of V-ATPase activity; (b) reduction or suppression of bone resorption and/or remodeling; (c) reduction or suppression of osteopetrosis or osteoporosis; (d) inhibition of bone resorption in vitro; (e) inhibition of renal brush border V-ATPase; (f) inhibition of macrophage microsome V-ATPase; (g) less potency in inhibiting lysosomal V-ATPase compared to osteoclast V-ATPase; (h) reduction or blocking of LDL metabolism in cells, which depends on acidification of lysosome; (i) reduction in V-ATPase mediated secretion; (j) reduction or suppression of tumor metastasis; (k) inhibition of intra-organellar acidification of intracellular organelles; (l) inhibition of urinary acidification; (m) inhibition of fertility; (n) inhibition of drug-resistance of tumor cells; (o) inhibition of tumor cell proliferation; (p) inhibition of cellular invasiveness; (q) reduction or suppression of gastric acidification and (R) inhibition of angiogenesis.

A beneficial effect can be a statistically significant effect in terms of statistical analysis of an effect of a hydrazide compound or a composition of the invention, versus the effects without the compound or composition that is not within the scope of the invention. Statistically significant” or “significantly different” effects or levels may represent levels that are higher or lower than a standard. In aspects of the invention, the difference may be 1.5, 2, 3, 4, 5, 6, 7, 8, 9, 10, 15, 20, 25 or 50 times higher or lower compared with the effect obtained without a hydrazide compound or a composition of the invention.

In aspects, the beneficial effect is a “sustained beneficial effect” where the beneficial effect is sustained for a prolonged period of time after termination of treatment. A treatment can be sustained over several days, weeks, months or years thereby having a major beneficial impact on the severity of the disease or disorder and its complications. In aspects of the invention, a beneficial effect may be sustained for a prolonged period of at least about 1 to 3 days, 2 to 4 weeks, 2 to 5 weeks, 3 to 5 weeks, 2 to 6 weeks, 2 to 8 weeks, 2 to 10 weeks, 2 to 12 weeks, 2 to 14 weeks, 2 to 16 weeks, 2 to 20 weeks, 2 to 24 weeks, 2 weeks to 12 months, 2 weeks to 18 months, 2 weeks to 24 months, or several years following treatment. The period of time a beneficial effect is sustained may correlate with the duration and timing of the treatment. A subject may be treated continuously for about or at least about 1 to 3 days, 1 week, 2 to 4 weeks, 2 to 6 weeks, 2 to 8 weeks, 2 to 10 weeks, 2 to 12 weeks, 2 to 14 weeks, 2 to 16 weeks, 2 weeks to 6 months, 2 weeks to 12 months, 2 weeks to 18 months, or several years, periodically or continuously.

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. Examples of carriers etc. include but are not limited to saline, buffered saline, dextrose, water, glycerol, ethanol, and combinations thereof. The use of such media and agents for an active substance is well known in the art.

“Therapeutically effective amount” relates to the amount or dose of a hydrazide compound or composition of the invention that will lead to one or more desired effects, in particular, one or more beneficial effects, more particularly therapeutic effects. A therapeutically effective amount of a substance can vary according to factors such as the disorder state, age, sex, and weight of the subject, and the ability of the substance to elicit a desired response in the subject. A dosage regimen may be adjusted to provide the optimum therapeutic response (e.g., beneficial effects). For example, several divided doses may be administered daily or the dose may be proportionally reduced as indicated by the exigencies of the therapeutic situation. A therapeutically effect amount can be a V-ATPase-inhibiting effective amount. A V-ATPase “inhibiting-effective amount,” as utilized in accordance with a compound, composition and method of the present invention, includes the dose necessary to achieve a V-ATPase “inhibiting-effective level” of the active compound in an individual patient. The V-ATPase inhibiting-effective amount can be defined, for example, as that amount required to be administered to a subject to achieve a V-ATPase inhibiting-effective blood level, tissue level, and/or intracellular level of a hydrazide compound of the present invention to effect the desired medical treatment.

“Disorder(s)” and “disease(s)” are used interchangeably herein and include a condition involving disruptions to normal V-ATPase activity and/or function, requiring modulation of V-ATPase-mediated secretion; or requiring regulation of biological phenomena including, but not limited to: intra-organellar acidification of intracellular organelles; urinary acidification; gastric acidification, bone resorption; fertility; angiogenesis; cellular invasiveness (e.g., tumor cell invasiveness); tumor cell proliferation and metastasis; and the development of drug resistance in tumor cells.

Examples of disorders and diseases encompassed by the present invention include without limitation osteoporosis associated with the peri and post menopausal conditions; Paget's disease; hypercalcemia associated with bone neoplasms and all the types of osteoporotic diseases including without limitation primary osteoporosis, involutional, Type I or postmenopausal, Type II or senile, juvenile, idiopathic in young adults, secondary osteoporosis, endocrine abnormality, hyperthyroidism, hypogonadism, ovarian agenesis or Turner's syndrome, hyperadrenocorticism or Cushing's syndrome, hyperparathyroidism, bone marrow abnormalities, multiple myeloma and related disorders, systemic mastocytosis, disseminated carcinoma, Gaucher's disease, connective tissue abnormalities, Osteogenesis imperfecta, homocystinuria, Ehlers-Danlos syndrome, Marfan's syndrome, Menke's syndrome, immobilisation or weightlessness, Sudeck's atrophy, chronic obstructive pulmonary disease, chronic alcoholism, chronic heparin administration, chronic ingestion of anticonvulsant drugs. The invention also encompasses the following diseases or disorders: tumours, in particular tumours related to renal cancer, melanoma, colon cancer, lung cancer and leukemia, renal tubular acidosis, viral conditions (for example those related to Semliki Forest virus, Vesicular Stomatitis virus, Newcastle Disease virus, Influenza A and B viruses, HIV virus), ulcers (for example chronic gastritis and peptic ulcer induced by Helicobacter pylori), autoimmune diseases and transplantation (for use as immunosuppressant agents), glaucoma, abnormal urinary acidification, hypercholesterolemic (for use as antilipidemic agents), atherosclerotic diseases, AIDS, neurodegenerative diseases such as Alzheimer's disease, angiogenic diseases including rheumatoid arthritis, diabetic retinopathy, psoriasis and solid tumours.

In embodiments, the disorders and diseases are those that can be controlled by the inhibition of V-ATPase including, for example, osteoporosis, Alzheimer's disease, glaucoma, and abnormal urinary acidification.

In embodiments of the invention, the disorder is a bone remodeling disorder selected from the group consisting of osteoporosis, Paget's disease, achondroplasia, osteochodrytis, hyperparathyroidism, osteogenesis imperfecta, congenital hypophosphatasia, fribromatous lesions, fibrous displasia, multiple myeloma, abnormal bone tomover, osteolytic bone disease, osteomalacia and periodontal disease. In particular embodiments, the bone disorder is osteoporosis, including primary osteoporosis, secondary osteoporosis, post-menopausal osteoporosis, male osteoporosis and steroid induced osteoporosis.

In embodiments of the invention, the disease or disorder is associated with loss of bone mass, such as osteoporosis and related osteopenic diseases.

In embodiments, the disorder is a disorder requiring increasing bone density in a subject for example a bone fracture, bone trauma, or a bone deficit condition associated with post-traumatic bone surgery, post-prosthetic joint surgery, post-plastic bone surgery, post-dental surgery, bone chemotherapy treatment or bone radiotherapy treatment.

In embodiments of the invention, the disorders or diseases are those which utilize an acid-promoted cell penetration mechanism. For example, the hydrazide compounds and compositions of the present invention can be used to inhibit the entry of viruses (e.g., baculoviruses and retroviruses), or to inhibit the entry of protein toxins (e.g., diphtheria toxin), into cells.

In embodiments, the hydrazide compounds and compositions of the present invention can be used to inhibit fertility in an animal, for example, a human, or to inhibit the proliferation, invasiveness or metastasis of tumor cells, or to promote the sensitivity of cancer toward drugs by inhibiting the ability of cancer cells to develop resistance to drugs, thereby facilitating and/or making possible the chemotherapeutic treatment of cancer.

In embodiments, the hydrazide compounds and compositions of the invention can be used for inhibiting cell survival and/or promoting cell death following exposure to cytotoxic agents (e.g. chemotherapeutic agents) and stress such as radiation or chemotherapy exposure. In a particular embodiment, the compounds may be used within or about 48 hours after the first exposure to the cytotoxic agent in an amount effective to prevent formation of acidic vesicular organelles in cells, thereby promoting cell death.

In embodiments, the hydrazide compounds and compositions of the invention can be used for promoting cell death of a cell which has been exposed to irradiation.

“Pharmaceutically acceptable salt(s),” means a salt that is pharmaceutically acceptable and has the desired pharmacological properties. By pharmaceutically acceptable salts is meant those salts which are suitable for use in contact with the tissues of a subject or patient without undue toxicity, irritation, allergic response and the like, and are commensurate with a reasonable benefit/risk ratio. Pharmaceutically acceptable salts are described for example, in S. M. Berge, et al., J. Pharmaceutical Sciences, 1977, 66:1. Suitable salts include salts that may be formed where acidic protons in the compounds are capable of reacting with inorganic or organic bases. Suitable inorganic salts include those formed with alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N-methylglucamine, and the like. Suitable salts also include acid addition salts formed with inorganic acids (e.g. hydrochloric and hydrobromic acids) and organic acids (e.g. acetic acid, citric acid, maleic acid, and the alkane- and arene-sulfonic acids such as methanesulfonic acid and benezenesulfonic acid). When there are two acidic groups present, a pharmaceutically acceptable salt may be a mono-acid-mono-salt or a di-salt; and similarly where there are more than two acidic groups present, some or all of such groups can be salified.

The term “pure” in general means better than 90%, 92%, 93%, 94%, 95%, 96%, 97%, 98% or 99% pure, and “substantially pure” means a compound synthesized such that the compound, as made as available for consideration into a composition or therapeutic dosage of the invention, has only those impurities that can not readily nor reasonably be removed by conventional purification processes.

A ‘hydrazide compound” refers to a compound of the formula I as described or defined herein. In aspects of the invention, a hydrazide compound comprises a compound set out in Table 1. In other aspects of the invention, a hydrazide compound of the formula I does not include a compound set out in Table 1.

The term “solvate” means a physical association of a compound with one or more solvent molecules or a complex of variable stoichiometry formed by a solute (for example, a hydrazide compound of the invention) and a solvent, for example, water, ethanol, methanol or acetic acid. This physical association may involve varying degrees of ionic and covalent bonding, including hydrogen bonding. In certain instances, the solvate will be capable of isolation, for example, when one or more solvent molecules are incorporated in the crystal lattice of the crystalline solid. In general, the solvents selected do not interfere with the biological activity of the solute. Solvates encompass both solution-phase and isolatable solvates. Representative solvates include hydrates, ethanolates, methanolates, and the like. In particular aspects of the invention the solvates are water and methanol.

The term “hydrate” means a solvate wherein the solvent molecule(s) is/are H₂O, including, mono-, di-, and various poly-hydrates thereof. Solvates can be formed using various methods known in the art.

The term “prodrug” means a covalently-bonded derivative or carrier of the parent compound or active drug substance which undergoes at least some biotransformation prior to exhibiting its pharmacological effect(s). In general, such prodrugs have metabolically cleavable groups and are rapidly transformed in vivo to yield the parent compound, for example, by hydrolysis in blood, and generally include esters and amide analogs of the parent compounds. The prodrug is formulated with the objectives of improved chemical stability, improved patient acceptance and compliance, improved bioavailability, prolonged duration of action, improved organ selectivity, improved formulation (e.g., increased hydrosolubility), and/or decreased side effects (e.g., toxicity). In general, prodrugs themselves have weak or no biological activity and are stable under ordinary conditions. Prodrugs can be readily prepared from the parent compounds using methods known in the art, such as those described in A Textbook of Drug Design and Development, Krogsgaard-Larsen and H. Bundgaard (eds.), Gordon & Breach, 1991, particularly Chapter 5: “Design and Applications of Prodrugs”; Design of Prodrugs, H. Bundgaard (ed.), Elsevier, 1985; Prodrugs: Topical and Ocular Drug Delivery, K. B. Sloan (ed.), Marcel Dekker, 1998; Methods in Enzymology, K. Widder et al. (eds.), Vol. 42, Academic Press, 1985, particularly pp. 309 396; Burger's Medicinal Chemistry and Drug Discovery, 5th Ed., M. Wolff (ed.), John Wiley & Sons, 1995, particularly Vol. 1 and pp. 172 178 and pp. 949 982; Pro-Drugs as Novel Delivery Systems, T. Higuchi and V. Stella (eds.), Am. Chem. Soc., 1975; and Bioreversible Carriers in Drug Design, E. B. Roche (ed.), Elsevier, 1987.

The term “co-crystal” as used herein means a crystalline material comprised of two or more unique solids at room temperature, each containing distinctive physical characteristics, such as structure, melting point, and heats of fusion. Co-crystals can be formed by an active pharmaceutical ingredient (API) and a co-crystal former either by hydrogen bonding or other non-covalent interactions, such as pi stacking and van der Waals interactions. An aspect of the invention provides for a co-crystal wherein the co-crystal former is a second API. In another aspect, the co-crystal former is not an API. In another aspect, the co-crystal comprises more than one co-crystal former. For example, two, three, four, five, or more co-crystal formers can be incorporated in a co-crystal with an API. Pharmaceutically acceptable co-crystals are described, for example, in “Pharmaceutical co-crystals,” Journal of Pharmaceutical Sciences, Volume 95 (3) Pages 499-516, 2006. Methods for producing co-crystals are discussed in the United States Patent Application 20070026078.

A “polymer” refers to molecules comprising two or more monomer subunits that may be identical repeating subunits or different repeating subunits. A monomer generally comprises a simple structure, low-molecular weight molecule containing carbon. Polymers may optionally be substituted. Polymers that can be used in the present invention include without limitation vinyl, acryl, styrene, carbohydrate derived polymers, polyethylene glycol (PEG), polyoxyethylene, polymethylene glycol, poly-trimethylene glycols, polyvinylpyrrolidone, polyoxyethylene-polyoxypropylene block polymers, and copolymers, salts, and derivatives thereof. In aspects of the invention, the polymer is poly(2-acrylamido-2-methyl-1-propanesulfonic acid); poly(2-acrylamido-2-methyl,-1-propanesulfonic acid-coacrylonitrile, poly(2-acrylamido-2-methyl-1-propanesulfonic acid-co-styrene), poly(vinylsulfonic acid); poly(sodium 4-styrenesulfonic acid); and sulfates and sulfonates derived therefrom; poly(acrylic acid), poly(methylacrylate), poly(methyl methacrylate), and poly(vinyl alcohol). In embodiments of the invention, the polymer is a polyethylene glycol (PEG).

A “peptide” carrier includes one, two, three, four, or five or more amino acids covalently linked through a peptide bond. A peptide can comprise one or more naturally occurring amino acids, and analogs, derivatives, and congeners thereof A peptide can be modified to increase its stability, bioavailability, solubility, etc. “Peptide analogue” and “peptide derivative” as used herein include molecules which mimic the chemical structure of a peptide and retain the functional properties of the peptide. A carrier can be an amino acid such as alanine, glycine, proline, methionine, serine, threonine, histidine, asparagine, alanyl-alanyl, prolyl-methionyl, or glycyl-glycyl. A carrier can be a polypeptide such as albumin, antitrypsin, macroglobulin, haptoglobin, caeruloplasm, transferring, α- or β-lipoprotein, β- or γ-globulin or fibrinogen. A peptide can be attached to a hydrazide compound through a functional group on the side chain of certain amino acids (e.g. serine) or other suitable functional groups. A carrier may comprise four or more amino acids with groups attached to three or more of the amino acids through functional groups on side chains. In an aspect, the carrier is one amino acid, in particular a sulfonate derivative of an amino acid, for example cysteic acid.

The term “alkyl”, either alone or within other terms such as “thioalkyl” and “arylalkyl”, means a monovalent, saturated hydrocarbon radical which may be a straight chain (i.e. linear) or a branched chain. An alkyl radical for use in the present invention generally comprises from about 1 to 15 carbon atoms, particularly from about 1 to 10, 1 to 8 or 1 to 7, more particularly about 1 to 6 carbon atoms, or 1 to 4 carbon atoms. Illustrative alkyl radicals include methyl, ethyl, n-propyl, n-butyl, n-pentyl, n-hexyl, isopropyl, isobutyl, isopentyl, amyl, sec-butyl, tert-butyl, tert-pentyl, n-heptyl, n-octyl, n-nonyl, n-decyl, undecyl, n-dodecyl, n-tetradecyl, pentadecyl, n-hexadecyl, heptadecyl, n-octadecyl, nonadecyl, eicosyl, dosyl, n-tetracosyl, and the like, along with branched variations thereof. In certain aspects of the invention an alkyl radical is a C₁-C₆ lower alkyl comprising or selected from the group consisting of methyl, ethyl, n-propyl, n-butyl, n-pentyl, n-hexyl, isopropyl, isobutyl, isopentyl, amyl, tributyl, sec-butyl, tert-butyl, tert-pentyl, and n-hexyl. In certain aspects of the invention an alkyl radical is a C₁-C₄ lower alkyl comprising or selected from the group consisting of methyl, ethyl, n-propyl, n-butyl, isobutyl or sec-butyl. An alkyl radical may be optionally substituted with substituents as defined herein at positions that do not significantly interfere with the preparation of the hydrazide compounds and do not significantly reduce the efficacy of the compounds. In certain aspects of the invention, an alkyl radical is substituted with substituents, in particular one to five substituents, including halo, lower alkoxy, lower aliphatic, a substituted lower aliphatic, hydroxy, cyano, nitro, thio, amino, keto, aldehyde, ester, amide, substituted amino, carboxyl, sulfonyl, sulfinyl, sulfenyl, sulfate, sulfoxide, substituted carboxyl, halogenated lower alkyl (e.g. CF₃), halogenated lower alkoxy, hydroxycarbonyl, lower alkoxycarbonyl, lower alkylcarbonyloxy, or lower alkylcarbonylamino. Substituents on an alkyl group may themselves be substituted.

In aspects of the invention, “substituted alkyl” refers to an alkyl or an alkane possessing less than 6 carbons, preferably less than 4 carbons, where at least one of the hydrogen atoms has been replaced by a halogen, an amino, a hydroxy, a nitro, a thio, a ketone, an aldehyde, an ester, an amide, a lower aliphatic, or a substituted lower aliphatic. Examples of such groups include, but are not limited to, 1-chloromethyl and the like.

As used herein in respect to certain aspects of the invention, the term “lower-alkyl-substituted-amino” refers to any alkyl unit containing up to and including eight carbon atoms where one of the aliphatic hydrogen atoms is replaced by an amino group. Examples of such groups include, but are not limited to, ethylamino and the like.

As used herein in respect to certain aspects of the invention, the term “lower-alkyl-substituted-halogen” refers to any alkyl chain containing up to and including eight carbon atoms where one of the aliphatic hydrogen atoms is replaced by a halogen. Examples of such groups include, but are not limited to, chlorethyl and the like.

As used herein the term “alkenyl” refers to an unsaturated, acyclic branched or straight-chain hydrocarbon radical comprising at least one double bond. An alkenyl radical may contain from about 2 to 15, or 2 to 10 carbon atoms, in particular from about 3 to 8 carbon atoms and more particularly about 3 to 6 or 2 to 6 carbon atoms. Suitable alkenyl radicals include without limitation ethenyl, propenyl (e.g., prop-1-en-1-yl, prop-1-en-2-yl, prop-2-en-1-yl(allyl), and prop-2-en-2-yl), buten-1-yl, but-1-en-2-yl, 2-methyl-prop-1-en-1-yl, but-2-en-1-yl, but-2-en-2-yl, buta-1,3-dien-1-yl, buta-1,3-dien-2-yl, hexen-1-yl, 3-hydroxyhexan-1-yl, hepten-1-yl, and octen-1-yl, and the like. An alkenyl radical may be optionally substituted similar to alkyl.

In aspects of the invention, “substituted alkenyl” includes an alkenyl group substituted by, for example, one to three substituents, preferably one to two substituents, such as alkyl, alkoxy, haloalkoxy, alkylalkoxy, haloalkoxyalkyl, alkanoyl, alkanoyloxy, cycloalkyl, cycloalkoxy, acyl, acylamino, acyloxy, amino, alkylamino, alkanoylamino, aminoacyl, aminoacyloxy, cyano, halogen, hydroxyl, carboxyl, carboxylalkyl, carbamyl, keto, thioketo, thiol, alkylthio, sulfonyl, sulfonamido, thioalkoxy, nitro, and the like.

As used herein, the term “alkynyl” refers to an unsaturated, branched or straight-chain hydrocarbon radical comprising one or more triple bonds. An alkynyl radical may contain about 1 to 15, or 2-10 carbon atoms, particularly about 3 to 8 carbon atoms and more particularly about 3 to 6 carbon atoms. Suitable alkynyl radicals include without limitation ethynyl, such as prop-1-yn-1-yl and prop-2-yn-1-yl, butynyls such as but-1-yn-1-yl, but-1-yn-3-yl, and but-3-yn-1-yl, pentynyls such as pentyn-1-yl, pentyn-2-yl, 4-methoxypentyn-2-yl, and 3-methylbutyn-1-yl, hexynyls such as hexyn-1-yl, hexyn-2-yl, hexyn-3-yl, and 3,3-dimethylbutyn-1-yl radicals and the like. An alkynyl may be optionally substituted similar to alkyl.

In aspects of the invention, “substituted alkynyl” includes an alkynyl group substituted by, for example, a substituent, such as, alkyl, alkoxy, alkanoyl, alkanoyloxy, cycloalkyl, cycloalkoxy, acyl, acylamino, acyloxy, amino, alkylamino, alkanoylamino, aminoacyl, aminoacyloxy, cyano, halogen, hydroxyl, carboxyl, carboxylalkyl, carbamyl, keto, thioketo, thiol, alkylthio, sulfonyl, sulfonamido, thioalkoxy, nitro, and the like.

As used herein the term “alkylene” refers to a linear or branched radical having from about 1 to 10, 1 to 8, 1 to 6, or 2 to 6 carbon atoms and having attachment points for two or more covalent bonds. Examples of such radicals are methylene, ethylene, propylene, butylene, pentylene, hexylene, ethylidene, methylethylene, and isopropylidene.

As used herein the term “alkenylene” refers to a linear or branched radical having from about 2 to 10, 2 to 8, or 2 to 6 carbon atoms, at least one double bond, and having attachment points for two or more covalent bonds. Examples of alkenylene radicals include 1,1-vinylidene (—CH₂═C—), 1,2-vinylidene (—CH═CH—), and 1,4-butadienyl (—CH═CH—CH═CH—).

As used herein the term “halo” refers to a halogen such as fluorine, chlorine, bromine or iodine atoms.

As used herein the term “hydroxyl” or “hydroxy” refers to an —OH group.

As used herein the term “cyano” refers to a carbon radical having three of four covalent bonds shared by a nitrogen atom, in particular A cyano group may be substituted with substituents described herein.

As used herein the term “alkoxy” refers to a linear or branched oxy-containing radical having an alkyl portion of one to about ten carbon atoms, such as a methoxy radical, which may be substituted. In aspects of the invention an alkoxy radical may comprise about 1-10, 1-8, 1-6, or 1-3 carbon atoms. In embodiments of the invention, an alkoxy radical comprises about 1-6 carbon atoms and includes a C₁-C₆ alkyl-O-radical wherein C₁-C₆ alkyl has the meaning set out herein. In embodiments of the invention, an alkoxy radical comprises about 1-4 carbon atoms and includes a C₁-C₄ alkyl-O-radical wherein C₁-C₄ alkyl has the meaning set out herein. Examples of alkoxy radicals include without limitation methoxy, ethoxy, propoxy, butoxy, isopropoxy and tert-butoxy alkyls.

An “alkoxy” radical may optionally be substituted with one or more substitutents disclosed herein including alkyl atoms to provide “alkylalkoxy” radicals; halo atoms, such as fluoro, chloro or bromo, to provide “haloalkoxy” radicals (e.g. fluoromethoxy, chloromethoxy, trifluoromethoxy, difluoromethoxy, trifluoroethoxy, fluoroethoxy, tetrafluoroethoxy, pentafluoroethoxy, and fluoropropox) and “haloalkoxyalkyl” radicals (e.g. fluoromethoxymethyl, chloromethoxymethyl, trifluoromethoxymethyl, difluoromethoxy-ethyl, and trifluoroethoxymethyl).

As used herein the term “alkenyloxy” refers to linear or branched oxy-containing radicals having an alkenyl portion of about 2 to 10 carbon atoms, such as an ethenyloxy or propenyloxy radical. An alkenyloxy radical may be a “lower alkenyloxy” radical having about 2 to 6 carbon atoms. Examples of alkenyloxy radicals include without limitation ethenyloxy, propenyloxy, butenyloxy, and isopropenyloxy alkyls. An “alkenyloxy” radical may be substituted with one or more substitutents disclosed herein including halo atoms, such as fluoro, chloro or bromo, to provide “haloalkenyloxy” radicals (e.g. trifluoroethenyloxy, fluoroethenyloxy, difluoroethenyloxy, and fluoropropenyloxy).

As used herein, the term “cycloalkyl” refers to an optionally substituted, saturated hydrocarbon ring system containing 1 to 2 rings and 3 to 7 carbons per ring. Examples of cycloalkyl groups include single ring structures such as cyclopropyl, cyclobutyl, cyclopentyl, cyclohexyl, cycloheptyl, cyclooctyl, cyclononyl, cyclodecyl, cyclododecyl, and the like, or multiple ring structures such as adamantanyl, and the like. In certain aspects of the invention the cycloalkyl radicals are “lower cycloalkyl” radicals having from about 3 to 10, 3 to 8, 3 to 6, or 3 to 4 carbon atoms, in particular cyclopropyl, cyclobutyl, cyclopentyl, cyclohexyl and cycloheptyl. A cycloalkyl radical may be optionally substituted with groups as disclosed herein.

In aspects of the invention, “substituted cycloalkyl” includes cycloalkyl groups having from 1 to 5 (in particular 1 to 3) substituents including without limitation alkyl, alkenyl, alkoxy, cycloalkyl, substituted cycloalkyl, acyl, acylamino, acyloxy, amino, aminoacyl, aminoacyloxy, oxyacylamino, cyano, halogen, hydroxyl, carboxyl, carboxylalkyl, keto, thioketo, thiol, thioalkoxy, hydroxyamino, alkoxyamino, and nitro.

As used herein, the term “aryl”, alone or in combination, refers to a carbocyclic aromatic system containing one, two or three rings wherein such rings may be attached together in a pendant manner or may be fused. In aspects of the invention an aryl radical comprises 4 to 10, 4 to 8, or 4 to 6 carbon atoms. Illustrative “aryl” radicals includes without limitation aromatic radicals such as phenyl, benzyl, naphthyl, indenyl, benzocyclooctenyl, benzocycloheptenyl, pentalenyl, azulenyl, tetrahydronaphthyl, indanyl, biphenyl, acephthylenyl, fluorenyl, phenalenyl, phenanthrenyl, and anthracenyl. An aryl radical may be optionally substituted with groups as disclosed herein, in particular hydroxyl, alkyl (“arylalkyl”), carbonyl, carboxyl, thiol (“thioalkyl”), amino, and/or halo, in particular a substituted aryl includes without limitation arylamine and arylalkylamine.

In aspects of the invention, an aryl radical may be optionally substituted with one to four substituents such as alkyl, substituted alkyl, alkenyl, substituted alkenyl, alkynyl, substituted alkynyl, halo, hydroxy, alkoxy, alkanoyl, alkanoyloxy, aryloxy, aralkyloxy, amino, alkylamino, arylamino, aralkylamino, dialkylamino, alkanoylamino, thiol, alkylthio, ureido, nitro, cyano, carboxy, carboxyalkyl, carbamyl, alkoxycarbonyl, alkylthiono, arylthiono, arylsulfonylamine, sulfonic acid, alkysulfonyl, sulfonamido, and the like. A substituent may be further substituted by hydroxy, halo, alkyl, alkoxy, alkenyl, alkynyl, aryl or aralkyl. In aspects of the invention an aryl radical is substituted with hydroxyl, alkyl, carbonyl, carboxyl, thiol, amino, and/or halo. The term “aralkyl” refers to an aryl or a substituted aryl group bonded directly through an alkyl group, such as benzyl. Other particular examples of substituted aryl radicals include chlorobenzyl, and amino benzyl.

As used herein, the term “aryloxy” refers to aryl radicals, as defined above, attached to an oxygen atom. Exemplary aryloxy groups include napthyloxy, quinolyloxy, isoquinolizinyloxy, and the like.

As used herein the term “arylalkoxy,” refers to an aryl group attached to an alkoxy group. Representative examples of arylalkoxy groups include, but are not limited to, 2-phenylethoxy, 3-naphth-2-ylpropoxy, and 5-phenylpentyloxy.

As used herein the term “heteroaryl” refers to fully unsaturated heteroatom-containing ring-shaped aromatic radicals having at least one heteroatom selected from carbon, nitrogen, sulfur and oxygen. A heteroaryl radical may contain one, two or three rings and the rings may be attached in a pendant manner or may be fused. In aspects of the invention the term refers to fully unsaturated heteroatom-containing ring-shaped aromatic radicals having from 3 to 15, 3 to 10, 3 to 8, 5 to 15, 5 to 10, or 5 to 8 ring members selected from carbon, nitrogen, sulfur and oxygen, wherein at least one ring atom is a heteroatom. Examples of “heteroaryl” radicals, include without limitation, an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, tetrazolyl and the like; an unsaturated condensed heterocyclic group containing 1 to 5 nitrogen atoms, in particular, indolyl, isoindolyl, indolizinyl, indazolyl, quinazolinyl, pteridinyl, quinolizidinyl, phthalazinyl, naphthyridinyl, quinoxalinyl, cinnolinyl, phenanthridinyl, acridinyl, phenanthrolinyl, phenazinyl, carbazolyl, purinyl, benzimidazolyl, quinolyl, isoquinolyl, quinolinyl, isoquinolinyl, indazolyl, benzotriazolyl, tetrazolopyridazinyl and the like; an unsaturated 3 to 6-membered heteromonocyclic group containing an oxygen atom, in particular, 2-furyl, 3-furyl, pyranyl, and the like; an unsaturated 5 to 6-membered heteromonocyclic group containing a sulfur atom, in particular, thienyl, 2-thienyl, 3-thienyl, and the like; unsaturated 5 to 6-membered heteromonocyclic group containing 1 to 2 oxygen atoms and 1 to 3 nitrogen atoms, in particular, furazanyl, benzofurazanyl, oxazolyl, isoxazolyl, and oxadiazolyl; an unsaturated condensed heterocyclic group containing 1 to 2 oxygen atoms and 1 to 3 nitrogen atoms, in particular benzoxazolyl, benzoxadiazolyl and the like; an unsaturated 5 to 6-membered heteromonocyclic group containing 1 to 2 sulfur atoms and 1 to 3 nitrogen atoms, for example, thiazolyl, isothiazolyl, thiadiazolyl and the like; an unsaturated condensed heterocyclic group containing 1 to 2 sulfur atoms and 1 to 3 nitrogen atoms such as benzothiazolyl, benzothiadiazolyl and the like. The term also includes radicals where heterocyclic radicals are fused with aryl radicals, in particular bicyclic radicals such as benzofuranyl, benzothiophenyl, phthalazinyl, chromenyl, xanthenyl, and the like. A heteroaryl radical may be optionally substituted with groups as disclosed herein, for example with an alkyl, amino, halogen, etc., in particular a heteroarylamine.

A heteroaryl radical may be optionally substituted with groups disclosed herein, for example with an alkyl, amino, halogen, etc., in particular a substituted heteroaryl radical is a heteroarylamine.

The term “heterocyclic” refers to saturated and partially saturated heteroatom-containing ring-shaped radicals having at least one heteroatom selected from carbon, nitrogen, sulfur and oxygen. In an aspect, the term refers to a saturated and partially saturated heteroatom-containing ring-shaped radicals having from about 3 to 15, 3 to 10, 5 to 15, 5 to 10, or 3 to 8 ring members selected from carbon, nitrogen, sulfur and oxygen, wherein at least one ring atom is a heteroatom. Exemplary saturated heterocyclic radicals include without limitation a saturated 3 to 6-membered heteromonocyclic group containing 1 to 4 nitrogen atoms [e.g. pyrrolidinyl, imidazolidinyl, piperidinyl, and piperazinyl]; a saturated 5 to 6-membered heteromonocyclic group containing 1-2 oxygen atoms; a saturated 3 to 6-membered heteromonocyclic group containing 1 to 2 oxygen atoms and 1 to 3 nitrogen atoms [e.g. morpholinyl; sydnonyl]; and, a saturated 3 to 6-membered heteromonocyclic group containing 1 to 2 sulfur atoms and 1 to 3 nitrogen atoms [e.g., thiazolidinyl] etc. Examples of partially saturated heterocyclyl radicals include without limitation dihydrothiophene, dihydropyranyl, dihydrofuranyl and dihydrothiazolyl. Illustrative heterocyclic radicals include without limitation aziridinyl, azetidinyl, 2-pyrrolinyl, 3-pyrrolinyl, pyrrolidinyl, azepinyl, 1,3-dioxolanyl, 2H-pyranyl, 4H-pyranyl, piperidinyl, 1,4-dioxanyl, morpholinyl, pyrazolinyl, 1,4-dithianyl, thiomorpholinyl, 1,2,3,6-tetrahydropyridinyl, oxiranyl, oxetanyl, tetrahydrofuranyl, tetrahydropyranyl, tetrahydropyridinyl, tetrahydrothiopyranyl, thioxanyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3H-indolyl, quinuclidinyl, quinolizinyl, and the like.

The foregoing heteroaryl and heterocyclic groups may be C-attached or N-attached (where such is possible).

As used herein the term “sulfonyl”, used alone or linked to other terms such as alkylsulfonyl, refers to the divalent radicals —SO₂—. In aspects of the invention, the sulfonyl group may be attached to a substituted or unsubstituted hydroxyl, alkyl group, ether group, alkenyl group, alkynyl group, aryl group, cycloalkyl group, cycloalkenyl group, cycloalkynyl group, heterocyclic group, carbohydrate, peptide, or peptide derivative.

The term “sulfinyl”, used alone or linked to other terms such as alkylsulfinyl (i.e. —S(O)-alkyl), refers to the divalent radicals —S(O)—.

The term “sulfonate” is art recognized and includes a group represented by the formula:

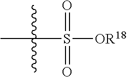

wherein R¹⁸ is an electron pair, hydrogen, alkyl, cycloalkyl, aryl, alkenyl, alkynyl, cycloalkenyl, cycloalkynyl, heterocyclic, carbohydrate, peptide, or peptide derivative.

The term “sulfate”, used alone or linked to other terms, is art recognized and includes a group that can be represented by the formula:

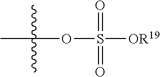

wherein R¹⁹ is an electron pair, hydrogen, alkyl, alkenyl, alkynyl, cycloalkyl, cycloalkenyl, cycloalkynyl, aryl, heterocyclic, carbohydrate, peptide or peptide derivative.

The term “sulfoxide” refers to the radical —S═O.

As used herein the term “amino”, alone or in combination, refers to a radical where a nitrogen atom (N) is bonded to three substituents being any combination of hydrogen, hydroxyl, alkyl, cycloalkyl, alkenyl, alkynyl, aryl, heterocyclic, or heteroaryl with the general chemical formula —NR²¹ where R²⁰ and R²¹ can be any combination of hydrogen, hydroxyl, alkyl, cycloalkyl, alkoxy, alkenyl, alkynyl, aryl, carbonyl carboxyl, amino, heteroaryl, or heterocyclic which may or may not be substituted. Optionally one substituent on the nitrogen atom may be a hydroxyl group (—OH) to provide an amine known as a hydroxylamine. Illustrative examples of amino groups are amino (—NH₂), alkylamino, acylamino, cycloamino, acycloalkylamino, arylamino, and arylalkylamino, in particular methylamino, ethylamino, dimethylamino, 2-propylamino, butylamino, isobutylamino, cyclopropylamino, benzylamino, allylamino, hydroxylamino, cyclohexylamino, piperidinyl, hydrazinyl, benzylamino, diphenylmethylamino, and tritylamino, which may or may not be substituted.

As used herein the term “thiol” means —SH. A thiol may be substituted with a substituent disclosed herein, in particular alkyl(thioalkyl), aryl(thioaryl), alkoxy (thioalkoxy) or carboxyl.

The term “sulfenyl” used alone or linked to other terms such as alkylsulfenyl, refers to the radical —SR²⁵ wherein R²⁵ is not hydrogen. In aspects of the invention R²⁵ is substituted or unsubstituted alkyl, cycloalkyl, alkenyl, alkynyl, aryl, heterocyclic, heteroaryl, carbonyl, carbamoyl, alkoxy, or carboxyl.

As used herein, the term “thioalkyl”, alone or in combination, refers to a chemical functional group where a sulfur atom (S) is bonded to an alkyl, which may be substituted. Examples of thioalkyl groups are thiomethyl, thioethyl, and thiopropyl. A thioalkyl may be substituted with a substituted or unsubstitute carboxyl, aryl, heterocylic, carbonyl, or heterocyclic.

As used herein the term “thioaryl”, alone or in combination, refers to a chemical functional group where a sulfur atom (S) is bonded to an aryl group with the general chemical formula —SR²⁶ where R²⁶ is aryl which may be substituted. Illustrative examples of thioaryl groups and substituted thioaryl groups are thiophenyl, chlorothiophenyl, para-chlorothiophenyl, thiobenzyl, 4-methoxy-thiophenyl, 4-nitro-thiophenyl, and para-nitrothiobenzyl.

As used herein the term “thioalkoxy”, alone or in combination, refers to a chemical functional group where a sulfur atom (S) is bonded to an alkoxy group with the general chemical formula —SR²⁷ where R²⁷ is an alkoxy group which may be substituted. A “thioalkoxy group” may have 1-6 carbon atoms i.e. a —S—(O)—C₁-C₆ alkyl group wherein C₁-C₆ alkyl have the meaning as defined above. Illustrative examples of a straight or branched thioalkoxy group or radical having from 1 to 6 carbon atoms, also known as a C₁-C₆ thioalkoxy, include thiomethoxy and thiomethoxy.

As used herein, the term “carbonyl” refers to a carbon radical having two of the four covalent bonds shared with an oxygen atom.

As used herein, the term “carboxyl”, alone or in combination, refers to —C(O)OR¹⁵— or —C(═O)OR¹⁵ wherein R¹⁵ is hydrogen, alkyl, alkenyl, alkynyl, cycloalkyl, cycloalkenyl, amino, thiol, aryl, heteroaryl, thioalkyl, thioaryl, thioalkoxy or a heterocyclic, which may optionally be substituted. In aspects of the invention, —C(O)OR¹⁵ provides an ester or an amino acid derivative. An esterified form is also particularly referred to herein as a “carboxylic ester”. In aspects of the invention a “carboxyl” may be substituted, in particular substituted with alkyl which is optionally substituted with one or more of amino, amino, halo, alkylamino, aryl, carboxyl or a heterocyclic. Examples of carboxyl groups are methoxycarbonyl, butoxycarbonyl, tert.alkoxycarbonyl such as tert.butoxycarbonyl, arylmethoxycarbonyl having one or two aryl radicals including without limitation phenyl optionally substituted by for example lower alkyl, lower alkoxy, hydroxyl, halo, and/or nitro, such as benzyloxycarbonyl, methoxybenzyloxycarbonyl, diphenylmethoxycarbonyl, 2-bromoethoxycarbonyl, 2-iodoethoxycarbonyltert.buty-lcarbonyl, 4-nitrobenzyloxycarbonyl, diphenylmethoxy-carbonyl, benzhydroxycarbonyl, di-(4-methoxyphenyl-methoxycarbonyl, 2-bromoethoxycarbonyl, or 2-iodoethoxy-carbonyl. In aspects of the invention, the carboxyl group may be an alkoxy carbonyl, in particular methoxy carbonyl, ethoxy carbonyl, isopropoxy carbonyl, t-butoxycarbonyl, t-pentyloxycarbonyl, or heptyloxy carbonyl, especially methoxy carbonyl or ethoxy carbonyl.

As used herein, the term “carbamoyl”, alone or in combination, refers to amino, monoalkylamino, dialkylamino, monocycloalkylamino, alkylcycloalkylamino, and dicycloalkylamino radicals, attached to one of two unshared bonds in a carbonyl group.

As used herein, the term “carboxamide” refers to the group —CONH—.

As used herein, the term “nitro” means —NO₂—.

As used herein, the term “acyl”, alone or in combination, means a carbonyl or thiocarbonyl group bonded to a radical selected from, for example, optionally substituted, hydrido, alkyl (e.g. haloalkyl), alkenyl, alkynyl, alkoxy (“acyloxy” including acetyloxy, butyryloxy, iso-valeryloxy, phenylacetyloxy, benzoyloxy, p-methoxybenzoyloxy, and substituted acyloxy such as alkoxyalkyl and haloalkoxy), aryl, halo, heterocyclyl, heteroaryl, sulfinyl (e.g. alkylsulfinylalkyl), sulfonyl (e.g. alkylsulfonylalkyl), cycloalkyl, cycloalkenyl, thioalkyl, thioaryl, amino (e.g. alkylamino or dialkylamino), and aralkoxy. Illustrative examples of “acyl” radicals are formyl, acetyl, 2-chloroacetyl, 2-bromoacetyl, benzoyl, trifluoroacetyl, phthaloyl, malonyl, nicotinyl, and the like.

In aspects of the invention, “acyl” refers to a group —C(O)R³⁰, where R³⁰ is hydrogen, alkyl, cycloalkyl, cycloheteroalkyl, aryl, arylalkyl, heteroalkyl, heteroaryl, and heteroarylalkyl. Examples include, but are not limited to formyl, acetyl, cyclohexylcarbonyl, cyclohexylmethylcarbonyl, benzoyl, benzylcarbonyl and the like.

As used herein the term “phosphonate” refers to a C—PO(OH)₂ or C—PO(OR³²)₂ group wherein R³² is alkyl or aryl which may be substituted.

As used herein, “ureido” refers to the group “—NHCONH—”. A ureido radical includes an alkylureido comprising a ureido substituted with an alkyl, in particular a lower alkyl attached to the terminal nitrogen of the ureido group. Examples of an alkylureido include without limitation N′-methylureido, N′-ethylureido, N′-n-propylureido, N′-1-propylureido and the like. A ureido radical also includes a N′,N′-dialkylureido group containing a radical —NHCON where the terminal nitrogen is attached to two optionally substituted radicals including alkyl, aryl, heterocylic, and heteroaryl.

The terms used herein for radicals including “alkyl”, “alkoxy”, “alkenyl”, “alkynyl”, “hydroxyl” etc. refer to both unsubstituted and substituted radicals. The term “substituted,” as used herein, means that any one or more moiety on a designated atom (e.g., hydrogen) is replaced with a selection from a group disclosed herein, provided that the designated atom's normal valency is not exceeded, and that the substitution results in a stable compound. Combinations of substituents and/or radicals are permissible only if such combinations result in stable compounds. “Stable compound” refers to a compound that is sufficiently robust to survive isolation to a useful degree of purity from a reaction mixture, and formulation into an efficacious therapeutic agent.

A radical in a hydrazide compound may be substituted with one or more substituents apparent to a person skilled in the art, including without limitation, alkyl, alkoxy, alkenyl, alkynyl, alkanoyl, alkylene, alkenylene, hydroxyalkyl, haloalkyl, haloalkylene, haloalkenyl, alkoxy, alkenyloxy, alkenyloxyalkyl, alkoxyalkyl, aryl, alkylaryl, haloalkoxy, haloalkenyloxy, heterocyclic, heteroaryl, alkylsulfonyl, sulfinyl, sulfonyl, sulfenyl, alkylsulfinyl, aralkyl, heteroaralkyl, cycloalkyl, cycloalkenyl, cycloalkoxy, cycloalkenyloxy, amino, oxy, halo, azido, thio, ═O, ═S, cyano, hydroxyl, phosphonato, phosphinato, thioalkyl, alkylamino, acylamino, arylsulfonyl, alkylcarbonyl, arylcarbonyl, aryloxy, aroyl, aralkanoyl, aralkoxy, aryloxyalkyl, haloaryloxyalkyl, and acyl groups. These substitutents may themselves be substituted.

### Hydrazide Compounds

A ‘hydrazide compound” includes a compound of the formula I

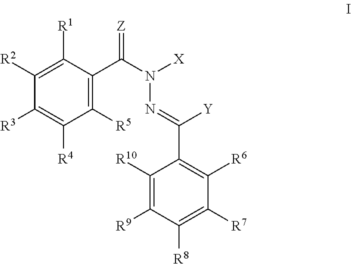

wherein
X is hydrogen or optionally substituted alkyl;
Y is hydrogen or optionally substituted alkyl, halo or alkoxy;

### Z is ═O, ═S; and

R¹, R², R³, R⁴, R⁵, R⁶, R⁷, R⁸, R⁹, and R¹⁰ are independently hydrogen, optionally substituted hydroxyl, alkyl, alkenyl, alkynyl, alkylene, alkenylene, alkoxy, alkenyloxy, cycloalkyl, cycloalkenyl, cycloalkynyl, cycloalkoxy, aryl, aryloxy, arylalkoxy, aroyl, heteroaryl, heterocyclic, acyl, acyloxy, sulfonyl, sulfinyl, sulfenyl, sulfoxide, sulfate, sulfonate, amino, imino, azido, thiol, thioalkyl, thioalkoxy, thioaryl, nitro, ureido, cyano, halo, ═O, ═S, phosphonate, carboxyl, carbonyl, carbamoyl, or carboxamide, or R⁶ and R⁷ may form an aryl, cycloalkyl, heteroaryl or heterocyclic ring; or an isomer or a pharmaceutically acceptable salt thereof.

In embodiments of the invention, a hydrazide compound of the formula I is provided wherein X is hydrogen or optionally substituted alkyl; Y is hydrogen or optionally substituted alkyl, halo or alkoxy; Z is ═O, ═S; and R¹, R², R³, R⁴, R⁵, R⁶ R⁷, R⁸, R⁹, and R¹⁰ are independently hydrogen, optionally substituted hydroxyl, alkyl, alkenyl, alkynyl, alkylene, alkenylene, alkoxy, alkenyloxy, acyl, acyloxy, sulfonyl, sulfinyl, sulfenyl, sulfoxide, sulfate, sulfonate, amino, imino, azido, thiol, nitro, ureido, cyano, halo, carboxyl, carbonyl, carbamoyl, or carboxamide; or R⁶ and R⁷ may form an aryl, cycloalkyl, heteroaryl or heterocyclic ring; or an isomer or a pharmaceutically acceptable salt thereof.

In an aspect, the invention provides a compound of the Formula I wherein X is hydrogen.

In an aspect, the invention provides a compound of the Formula I wherein X is alkyl.

In an aspect, the invention provides a compound of the Formula I wherein Y is hydrogen.

In an aspect, the invention provides a compound of the Formula I wherein Y is alkyl.

In an aspect, the invention provides a compound of the Formula I wherein Y is C₁-C₄ alkyl.

In an aspect, the invention provides a compound of the Formula I wherein Y is halo, in particular fluorine or chlorine.

In an aspect, the invention provides a compound of the Formula I wherein Y is alkoxy, in particular methoxy or ethoxy.

In an aspect, the invention provides a compound of the Formula I wherein Z is oxygen.

In an embodiments, the invention provides compounds of the formula I wherein X is hydrogen, Y is hydrogen and Z is ═O.

In an aspect, the invention provides a compound of the Formula I wherein at least one of R¹, R², R³, R⁴, R⁵, R⁶, R⁷, R⁸, R⁹, and R¹⁰ is optionally substituted alkyl, alkoxy, halo, nitro, or carboxyl.

In an aspect, the invention provides a compound of the Formula I wherein at least two of R¹, R², R³, R⁴, R⁵, R⁶, R⁷, R⁸, R⁹, and R¹⁰ is optionally substituted alkyl, alkoxy, halo, nitro, or carboxyl.

In an aspect, the invention provides a compound of the Formula I wherein at least one, two or three of R¹, R², R³, R⁴, R⁵, R⁶ R⁷, R⁸, R⁹, and R¹⁰ is optionally substituted hydroxyl, O-alkyl or alkoxy, O-aryl or aryloxy, or halo.

In an aspect, the invention provides a compound of the Formula I wherein 1 to 3 of R¹, R², R³, R⁴, and R⁵ are optionally substituted hydroxyl, alkoxy or halo.

In an aspect, the invention provides a compound of the Formula I wherein 1 to 3 of R⁶, R⁷, R⁸, R⁹, and R¹⁰ are optionally substituted hydroxyl, alkoxy or halo.

In an aspect, the invention provides a compound of the Formula I wherein 1 or 2 of R¹, R², R³, R⁴, and R⁵ are optionally substituted hydroxyl, alkoxy or halo.

In an aspect, the invention provides a compound of the Formula I wherein 1 or 2 of R⁶, R⁷, R⁸, R⁹, and R¹⁰ are optionally substituted hydroxyl, alkoxy or halo.

In an aspect, the invention provides a compound of the Formula I wherein 1 to 3 of R¹, R², R³, R⁴, and R⁵ are optionally substituted alkoxy or halo.

In an aspect, the invention provides a compound of the Formula I wherein 1 to 3 of R⁶, R⁷, R⁸, R⁹, and R¹⁰ are optionally substituted alkoxy or halo.

In an aspect, the invention provides a compound of the Formula I wherein 1 to 3 of R¹, R², R³, R⁴, and R⁵ are optionally substituted alkoxy, in particular methoxy, ethoxy or propoxy.

In an aspect, the invention provides a compound of the Formula I wherein 1 to 3 of R⁶, R⁷, R⁸, R⁹, and R¹⁰ are optionally substituted alkoxy, in particular methoxy, ethoxy or propoxy.

In an aspect, the invention provides a compound of the Formula I wherein 1 to 3 of R¹, R², R³, R⁴, and R⁵ are halo, in particular fluorine or chlorine.

In an aspect, the invention provides a compound of the Formula I wherein 1 to 3 of R⁶, R⁷, R⁸, R⁹, and R¹⁰ are halo, in particular fluorine or chlorine.

In an aspect, the invention provides a compound of the Formula I wherein 1 or 2 of R¹, R², R³, R⁴, and R⁵ are carboxyl.

In an aspect, the invention provides a compound of the Formula I wherein 1 or 2 of R⁶, R⁷, R⁸, R⁹, and R¹⁰ are carboxyl.

In an aspect, the invention provides a compound of the Formula I wherein 1 or 2 of R¹, R², R³, R⁴, and R⁵ are nitro.

In an aspect, the invention provides a compound of the Formula I wherein 1 or 2 of R⁶, R⁷, R⁸, R⁹, and R¹⁰ are nitro.

In an aspect, the invention provides a compound of the formula I wherein X is hydrogen, Y is hydrogen, Z is ═O, R¹ to R¹⁰ are independently optionally substituted hydroxyl, O-alkyl or alkoxy, O-aryl or aryloxy, or halo.

In an aspect, a compound of the formula I is provided wherein (a) 1 to 3 of R¹, R², R³, R⁴, and R⁵ are optionally substituted hydroxyl, alkoxy or halo, Z is ═O, X is hydrogen and Y is hydrogen or alkyl; (b) 1 to 3 of R⁶, R⁷, R⁸, R⁹, and R¹⁰ are optionally substituted hydroxyl, alkoxy or halo, Z is ═O, X is hydrogen and Y is hydrogen or alkyl; (c) 1 to 3 of R¹, R², R³, R⁴, and R⁵ are optionally substituted hydroxyl, alkoxy or halo, 1 to 3 of R⁶, R⁷, R⁸, R⁹, and R¹⁰ are optionally substituted hydroxyl, alkoxy or halo, Z is ═O, X is hydrogen and Y is hydrogen or alkyl.

In another aspect of the invention a compound of the Formula I is provided wherein 1 to 3 of R¹, R², R³, R⁴, and R⁵ are optionally substituted C₁-C₄ alkyl, F or Br, —NH₂, —NO₂, —CN, COOH, or C₁-C₄ alkoxy. In a particular aspect one or both of R³ and R⁴ are optionally substituted C₁-C₄ alkyl, F or Br, —NH₂, —NO₂, —CN, COOH, or C₁-C₄ alkoxy

In another aspect of the invention a compound of the Formula I is provided wherein 1 or 2 of R⁶, R⁷, R⁸, R⁹, and R¹⁰ are optionally substituted C₁-C₄ alkyl, F or Br, —NH₂, —NO₂, —CN, COOH, or C₁-C₄ alkoxy. In a particular aspect of the invention a compound of the Formula I is provided wherein 1 or 2 of R⁶, R⁷ and R¹⁰ are optionally substituted C₁-C₄ alkyl, F or Br, —NH₂, —NO₂, —CN, COOH, or C₁-C₄ alkoxy.

In another aspect of the invention a compound of the Formula I is provided wherein R⁶ and R⁷ form an aryl group, in particular a phenyl group.

In another aspect of the invention a compound of the Formula I is provided wherein R⁶ and R⁷ form a heterocyclic group, in particular a 5-6 membered monocyclic ring with 1-2 oxygen or nitrogen atoms.

In another aspect of the invention a compound of the Formula I is provided wherein 1 or 2 of R⁶, R⁷, R⁸, R⁹, and R¹⁰ are propoxy, —OCH₂CN, —OCH₂CH═CH₂, —CH₂CH═CH₂ and —OCH₂CHOHCH₂NHPrO.

In a particular aspect of the invention a compound of the Formula I is provided wherein 1 or 2 of R⁶, R⁷, R⁸, R⁹, and R¹⁰ are —OPR-n, —OPR-i, —OCH₂CN, —OCH₂CH═CH₂, and —CH₂CH═CH₂.

In aspects of the invention, a hydrazide compound comprises a compound set out in Table 1.

In other aspects of the invention, a hydrazide compound is a compound of the formula I and does not include a compound set out in Table 1.

In embodiments, the hydrazide compound is a compound of the formula II (compound 1 in Table 1, 3,4-dihydroxy-N′-(2-hydroxybenzylidene)benzohydrazide):

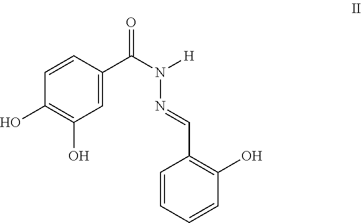

A compound of the formula II can provide a practical template that can be used to produce a vast number of structurally diverse, yet synthetically accessible, vacuolar-type (H+)-ATPase inhibitors and therapeutic compounds. Thus, the invention includes a functional derivative of a compound of the formula II. A “functional derivative” refers to a compound that possesses a biological activity (either functional or structural) that is substantially similar to the biological activity of a compound of the formula II. The term “functional derivative” is intended to include “variants” “analogs” or “chemical derivatives” of a compound of the formula II. The term “variant” is meant to refer to a molecule substantially similar in structure and function to a compound of the formula II or a part thereof. A molecule is “substantially similar” to a compound of the formula II if both molecules have substantially similar structures or if both molecules possess similar biological activity. The term “analog” refers to a molecule substantially similar in function to a compound of the formula II. The term “chemical derivative” describes a molecule that contains additional chemical moieties which are not normally a part of the base molecule.

Hydroxyls or carbons in compounds of the formula II may be substituted with one or more radicals disclosed herein, including without limitation, alkyl, alkenyl, alkynyl, alkylene, alkenylene, alkoxy, alkenyloxy, acyl, acyloxy, sulfonyl, sulfinyl, sulfenyl, to sulfoxide, sulfate, sulfonate, amino, imino, azido, thiol, nitro, ureido, cyano, halo, carboxyl, carbonyl, carbamoyl, or carboxamide.

A hydrazide compound of the invention also includes pharmaceutically acceptable salt(s) of a compound of the formula I or II. In aspects of the invention, an acid addition salt, in particular a halide salt, of a compound of the formula I or II is provided.

A hydrazide compound, in particular a compound of the Formula I or II contains one or more asymmetric centers and may give rise to enantiomers, diasteriomers, and other stereoisomeric forms which may be defined in terms of absolute stereochemistry as (R)— or (S)—. Thus, hydrazide compounds include all possible diasteriomers and enantiomers as well as their racemic and optically pure forms. Optically active (R)- and (S)-isomers may be prepared using chiral synthons or chiral reagents, or resolved using conventional techniques. When a hydrazide compound contains centers of geometric asymmetry, and unless specified otherwise, it is intended that the compounds include both E and A geometric isomers. All tautomeric forms are also included within the scope of a hydrazide compound employed in the present invention.

Solvates of the compounds of the formula I or II formed with water or common organic solvents are intended to be encompassed within the present invention. Thus, a hydrazide compound, in particular a compound of the formula I or II can exist in unsolvated as well as solvated forms with pharmaceutically acceptable solvents such as water, methanol, ethanol, and the like. The solvated forms may be considered equivalent to the unsolvated forms for the purposes of the present invention. In addition, hydrate forms of the hydrazide compounds and their salts are encompassed within this invention.

The amount of solvent used to make solvates can be determined by routine testing. For example, a monohydrate of a compound of the formula I or II can have about 1.5 equivalent of H₂O or 0.5 equivalent of methanol for each equivalent of a compound of the invention. However, more or less solvent may be used depending on the choice of solvate desired.

In particular embodiments, a solvate of a hydrazide compound comprises two compounds of the formula II, three water molecules and one methanol molecule.

Dehydrate, co-crystals, anhydrous, or amorphous forms of the hydrazide compounds are also encompassed in the present invention. Crystalline compounds of the formula I or II can be in the form of a free base, a salt, or a co-crystal. Free base compounds can be crystallized in the presence of an appropriate solvent in order to form a solvate. Acid salt compounds of the formula I or II (e.g. HCl, HBr, benzoic acid) can also be used in the preparation of solvates. For example, solvates can be formed by the use of acetic acid or ethyl acetate. The solvate molecules can form crystal structures via hydrogen bonding, van der Waals forces, or dispersion forces, or a combination of any two or all three forces.

Hydrazide compounds may be amorphous or may have different crystalline polymorphs, possibly existing in different solvation or hydration states. By varying the form of a drug, it is possible to vary the physical properties thereof. For example, crystalline polymorphs typically have different solubilities from one another, such that a more thermodynamically stable polymorph is less soluble than a less thermodynamically stable polymorph. Pharmaceutical polymorphs can also differ in properties such as shelf-life, bioavailability, morphology, vapor pressure, density, color, and compressibility.

Hydrazide compounds can include pharmaceutically acceptable co-crystals or co-crystal salts. A pharmaceutically acceptable co-crystal includes a co-crystal that is suitable for use in contact with the tissues of a subject or patient without undue toxicity, irritation, allergic response and has the desired pharmacokinetic properties. A co-crystal former which is generally a pharmaceutically acceptable compound, may be, for example, benzoquinone, terephthalaldehyde, saccharin, nicotinamide, acetic acid, formic acid, butyric acid, trimesic acid, 5-nitroisophthalic acid, adamantane-1,3,5,7-tetracarboxylic acid, formamide, succinic acid, fumaric acid, tartaric acid, malic acid, malonic acid, benzamide, mandelic acid, glycolic acid, fumaric acid, maleic acid, urea, nicotinic acid, piperazine, p-phthalaldehyde, 2,6-pyridinecarboxylic acid, 5-nitroisophthalic acid, citric acid, and the alkane- and arene-sulfonic acids such as methanesulfonic acid and benezenesulfonic acid.

A hydrazide compound may be in the form of a prodrug that is converted in vivo to an active compound. In a compound of the Formula I one or more radical may comprise a cleavable group that is cleaved after administration to a subject to provide an active (e.g., therapeutically active) compound, or an intermediate compound that subsequently yields the active compound. A cleavable group can be an ester that is removed either enzymatically or non-enzymatically. Examples of prodrugs include, but are not limited to esters (e.g., acetate, formate, and benzoate derivatives), carbamates (e.g. N,N-dimethylaminocarbonyl) of hydroxy functional groups on compounds of the formula I or II and the like.

In general, all physical forms of hydrazide compounds are intended to be within the scope of the present invention.

A hydrazide compound, in particular a compound of the formula I or II, may optionally comprise a carrier interacting with one or more radicals in the compound. A carrier may be a polymer or peptide, or derivatives or combinations thereof, and it may be optionally substituted, for example, with one or more alkyl, halo, hydroxyl, halo, or amino. A carrier may be directly or indirectly covalently attached to a hydrazide compound. A carrier may be substituted with substituents described herein including without limitation one or more alkyl, amino, nitro, halogen, thiol, thioalkyl, sulfate, sulfonyl, sulfinyl, sulfoxide and hydroxyl groups. In aspects of the invention the carrier is a polymer, in particular a PEG. In aspects of the invention the carrier is an amino acid including alanine, glycine, praline, methionine, serine, threonine, asparagine, alanyl-alanyl, prolyl-methionyl, or glycyl-glycyl. A carrier can also include a molecule that targets a hydrazide compound, in particular a compound of the formula I or II to a particular tissue or organ. Thus, a carrier may facilitate or enhance transport of a hydrazide compound, in particular a compound of the formula I or II to a target therapeutic site.

Hydrazide compounds of the present invention may be obtained by one of ordinary skill in the art by isolation from natural sources; chemical synthesis using well-known and readily available chemical reactions, reagents, and procedures; by semisynthesis; or the like.

Hydrazide compounds, in particular compounds of the Formula I or II can be prepared using reactions and methods generally known to the person of ordinary skill in the art, having regard to that knowledge and the disclosure of this application. The reactions are performed in a solvent appropriate to the reagents and materials used and suitable for the reactions being effected. It will be understood by those skilled in the art of organic synthesis that the functionality present on the compounds should be consistent with the proposed reaction steps. This will sometimes require modification of the order of the synthetic steps or selection of one particular process scheme over another in order to obtain a desired hydrazide compound. It will also be recognized that another major consideration in the development of a synthetic route is the selection of the protecting group used for protection of the reactive functional groups present in the compounds. An authoritative account describing the many alternatives to the skilled artisan is Greene and Wuts (Protective Groups In Organic Synthesis, Wiley and Sons, 1991).

Analogs and derivatives of a hydrazide compound can be prepared using conventional procedures. For example, one or more hydroxyl groups of a compound of the formula II can be converted to an ester (e.g., CO₂R,′) by reaction with a suitable esterifying agent such as an anhydride (e.g., (R′(CO)₂O) or an acid chloride (e.g., R′(CO)Cl), or the like, or converted to a sulfonate (e.g., SO₂R′) by reaction with a suitable sulfonating agent such as a sulfonyl chloride (e.g., R′SO₂Cl), or the like, wherein R′ is any suitable substituent including, for example, a substituent described herein.

One or more hydroxyl groups of a compound of the formula II may be converted to a halogen atom using a suitable halogenating agent such as an N-halosuccinimide such as N-iodosuccinimide, N-bromosuccinimide, N-chlorosuccinimide or the like, in the presence of an appropriate activating agent (e.g., a phosphine or the like). One or more hydroxyl groups may also be converted to an ether by reacting one or more hydroxyls with an alkylating agent in the presence of a suitable base. Examples of appropriate alkylating agents include, without limitation, an alkyl or aryl sulfonate, an alkyl or aryl halide, or the like. One or more suitably activated hydroxyls may also be converted to the corresponding thiol, cyano, halo, or amino derivative by displacement with a nucleophile. Examples of suitable nucleophiles include, without limitation, a thiol, a cyano, a halide ion, an amine (e.g., NH₂R″, wherein R″ is as described herein), or the like.

Functional groups such as, for example, amines can be obtained by a variety of methods known in the art. Amines can be obtained by hydrolysis of one or more amides or by reacting one or more suitable oxo groups (e.g., an aldehyde or a ketone) with one or more appropriate amines under suitable conditions (e.g., reductive animation conditions, or the like). Amines, in turn, can be converted to a number of other useful derivatives, including without limitation, amides, sulfonamides and the like. Other modifications may be achieved by incorporating synthetic, semisynthetic or naturally occurring materials including without limitation, one or more amino acids, into the structure of one or more hydrazide compounds. Other synthetic modifications or transformations can be accomplished, other than those described herein, using routine chemistry well known in the art.

The starting materials and reagents used in preparing the hydrazide compounds are either available from commercial suppliers or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

The starting materials, intermediates, and hydrazide compounds may be isolated and purified using conventional techniques, such as precipitation, filtration, distillation, crystallization, chromatography, and the like. The hydrazide compounds may be characterized using conventional methods, including physical constants and spectroscopic methods, in particular HPLC.

Hydrazide compounds which are basic in nature can form a wide variety of different salts with various inorganic and organic acids. In practice is it desirable to first isolate a hydrazide compound from the reaction mixture as a pharmaceutically unacceptable salt and then convert the latter to the free base compound by treatment with an alkaline reagent and subsequently convert the free base to a pharmaceutically acceptable acid addition salt. The acid addition salts of the base compounds of the hydrazide compounds are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

Hydrazide compounds which are acidic in nature are capable of forming base salts with various pharmacologically acceptable cations. These salts may be prepared by conventional techniques by treating the corresponding acidic compounds with an aqueous solution containing the desired pharmacologically acceptable cations and then evaporating the resulting solution to dryness, preferably under reduced pressure. Alternatively, they may be prepared by mixing lower alkanolic solutions of the acidic compounds and the desired alkali metal alkoxide together and then evaporating the resulting solution to dryness in the same manner as before. In either case, stoichiometric quantities of reagents are typically employed to ensure completeness of reaction and maximum product yields.

V-ATPase inhibitory activity of the hydrazide compounds of the present invention can be confirmed using any suitable method known in the art, for example, the assay methods disclosed herein. Suitable assay methods for measuring V-ATPase inhibitory activity are also described in the literature (see for example, Chan et al., Anal. Biochem., 157, 375-380 (1986); Bowman et al., Proc. Natl. Acad. Sci. USA, 85, 7972-7976 (1988); Drose et al., Biochemistry, 32, 3902-3906 (1993); Drose and Altendorf, J. Exp. Biol., 200, 1-8 (1997); Gagliardi et al., J. Med. Chem., 41, 1883-1893 (1998); Gagliardi et al., J. Med. Chem., 41, 1568-1573 (1998); Boyd, PCT International Patent Application No. PCT/US00/05582; and references cited therein). A compound may be selected based upon the level of V-ATPase inhibition, and/or a particular kind or location of intracellular organelle or plasma membrane V-ATPase selectively or preferentially inhibited by the compound. For example, the methods described herein are particularly useful for identifying compounds that selectively inhibit human osteoclast V-ATPase activity and therefore are expected to be particularly useful in treating bone disorders such as osteoporosis. A compound may also be selected based upon other pharmacological, toxicological, pharmaceutical or other pertinent considerations that are well-known to those skilled in the art.

Therapeutic efficacy of selected hydrazide compounds may be tested in in vitro and in vivo systems well-known to those skilled in the art. For example, the compounds may be tested in animal models of bone disease or cancer (see for example Niikura K, et al, J Toxicol Sci. 2005 December; 30(4):297-304; Niikura K., Med. Chem. 2009 July 24; Ostrov D A, et al, Cancer Chemother Pharmacol. 2007 September; 60(4):555-62. Epub 2006 December 23; Niikura K. et al, Bone. 2007 April; 40(4):888-94, Niikura K. et al, Br J. Pharmacol. 2004 June; 142(3):558-66.)

### Compositions and Kits

One or more hydrazide compound, in particular a compound of the formula I or II, may be formulated into a pharmaceutical composition for administration to a subject. Therefore, the invention provides formulations including without limitation pills, tablets, caplets, soft and hard gelatin capsules, lozenges, sachets, cachets, vegicaps, liquid drops, elixirs, suspensions, emulsions, solutions, syrups, aerosols (as a solid or in a liquid medium) suppositories, sterile injectable solutions, and/or sterile packaged powders, which contain a hydrazide compound in particular a pure or substantially pure hydrazide compound.

Pharmaceutical compositions of the present invention or fractions thereof comprise suitable pharmaceutically acceptable carriers, excipients, and vehicles selected based on the intended form of administration, and consistent with conventional pharmaceutical practices. Particular compositions of the invention may contain a hydrazide compound that is pure or substantially pure. Suitable pharmaceutical carriers, excipients, and vehicles are described in the standard text, Remington: The Science and Practice of Pharmacy (21st Edition. 2005, University of the Sciences in Philadelphia (Editor), Mack Publishing Company), and in The United States Pharmacopeia: The National Formulary (USP 24 NF19) published in 1999.

A composition of the invention may include at least one buffering agent or solution. Suitable buffering agents include, but are not limited to hydrochloric, hydrobromic, hydroiodic, sulfuric, phosphoric, formic, acetic, propionic, succinic, glycolic, glucoronic, maleic, furoic, citric, glutamic, benzoic, anthranilic, salicylic, phenylacetic, mandelic, embonic, pamoic, methanesulfonic, ethanesulfonic, pantothenic, benzenesulfonic, stearic, sulfanilic, algenic, galacturonic acid and mixtures thereof. Additional agents that may be included are one or more of pregelatinized maize starch, polyvinyl pyrrolidone, hydroxypropyl methylcellulose, lactose, microcrystalline cellulose, calcium hydrogen phosphate, magnesium stearate, talc, silica, potato starch, sodium starch glycolate, sodium lauryl sulfate, sorbitol syrup, cellulose derivatives, hydrogenated edible fats, lecithin, acacia, almond oil, oily esters, ethyl alcohol, fractionated vegetable oils, methyl, propyl-p-hydroxybenzoates, sorbic acid and mixtures thereof. Buffering agents may additionally comprise one or more of dichlorodifluoromethane, trichloro fluoromethane, dichlorotetra fluoroethane, carbon dioxide, poly (N-vinyl pyrrolidone), poly (methylmethacrylate), polylactide, polyglycolide and mixtures thereof. In some aspects, a buffering agent may be formulated as at least one medium including without limitation a suspension, solution, or emulsion. In other aspects, a buffering agent may additionally comprise a formulatory agent including without limitation a pharmaceutically acceptable carrier, excipient, suspending agent, stabilizing agent or dispersing agent.

In aspects of the invention, a pharmaceutical composition is provided for oral administration of one or more hydrazide compounds for treatment of a disease or disorder. By way of example for oral administration in the form of a capsule or tablet, the active component can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbital, and the like. For oral administration in a liquid form, the drug component may be combined with any oral, non-toxic, pharmaceutically acceptable inert carrier such as ethanol, glycerol, water, and the like. Suitable binders (e.g. gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions. Compositions as described herein can further comprise wetting or emulsifying agents, or pH buffering agents.

In aspects of the invention, a composition of the invention is a liquid solution, suspension, emulsion, tablet, pill, capsule, sustained release formulation, or powder. The compositions can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Various delivery systems are known and can be used to administer a composition of the invention, e.g. encapsulation in liposomes, microparticles, microcapsules, and the like.

Formulations for parenteral administration may include aqueous solutions, syrups, aqueous or oil suspensions and emulsions with edible oil such as cottonseed oil, coconut oil or peanut oil. Dispersing or suspending agents that can be used for aqueous suspensions include synthetic or natural gums, such as tragacanth, alginate, acacia, dextran, sodium carboxymethylcellulose, gelatin, methylcellulose, and polyvinylpyrrolidone.

Compositions for parenteral administration may include sterile aqueous or non-aqueous solvents, such as water, isotonic saline, isotonic glucose solution, buffer solution, or other solvents conveniently used for parenteral administration of therapeutically active agents. A composition intended for parenteral administration may also include conventional additives such as stabilizers, buffers, or preservatives, e.g. antioxidants such as methylhydroxybenzoate or similar additives.

Compositions of the invention can be formulated as pharmaceutically acceptable salts as described herein.

A hydrazide compound, in particular a compound of the formula I or II, or a composition of the invention may be sterilized by, for example, filtration through a bacteria retaining filter, addition of sterilizing agents to the compounds or composition, irradiation of the compounds or composition, or heating the compounds or composition. Alternatively, the compounds or compositions may be provided as sterile solid preparations e.g. lyophilized powder, which are readily dissolved in sterile solvent immediately prior to use.

After pharmaceutical compositions have been prepared, they can be placed in an appropriate container and labeled for treatment of an indicated condition. For administration of a composition of the invention, such labeling would include amount, frequency, and method of administration.

According to the invention, a kit is provided. In an aspect, the kit comprises a hydrazide compound or a formulation or composition of the invention in kit form. The kit can be a package which houses a container which contains hydrazide compounds or formulations/compositions of the invention and also houses instructions for administering the compounds or formulations/compositions to a subject. The invention further relates to a commercial package comprising hydrazide compounds or formulations/compositions of the invention together with instructions for their use. In particular a label may include amount, frequency, and method of administration.

The invention also provides a pharmaceutical pack or kit comprising one or more containers filled with one or more of the ingredients of a composition of the invention to provide a beneficial effect including a therapeutic effect. Associated with such container(s) can be various written materials such as instructions for use, or a notice in the form prescribed by a governmental agency regulating the labeling, manufacture, use or sale of pharmaceuticals or biological products, which notice reflects approval by the agency of manufacture, use, or sale for human administration.

The invention also relates to articles of manufacture and kits containing materials useful for treating a disease or disorder disclosed herein. An article of manufacture may comprise a container with a label. Examples of suitable containers include bottles, vials, and test tubes which may be formed from a variety of materials including glass and plastic. A container holds a hydrazide compound or formulations/compositions of the invention which are effective for treating a disease or disorder disclosed herein. The label on the container indicates that the hydrazide compounds or formulations/compositions of the invention are used for treating a disease or disorder disclosed herein and may also indicate directions for use. In aspects of the invention, a medicament or formulation in a container may comprise any of the medicaments or formulations disclosed herein.

In aspects of the invention, a kit of the invention comprises a container described herein. In particular aspects, a kit of the invention comprises a container described herein and a second container comprising a buffer. A kit may additionally include other materials desirable from a commercial and user standpoint, including, without limitation, buffers, diluents, filters, needles, syringes, and package inserts with instructions for performing any methods disclosed herein (e.g., methods for treating a disorder disclosed herein). A medicament or formulation in a kit of the invention may comprise any of the formulations or compositions disclosed herein.

In aspects of the invention, the kits may be useful for any of the methods disclosed herein, including, without limitation treating a subject suffering from a disease or disorder disclosed herein. Kits of the invention may contain instructions for practicing any of the methods described herein.

### Administration

A hydrazide compound and composition of the present invention can be administered by any means that produces contact of the active agent(s) with the agent's sites of action in the body of a subject or patient to produce a therapeutic effect, in particular a beneficial effect, in particular a sustained beneficial effect. A hydrazide compound or composition of the invention can be formulated for sustained release, for delivery locally or systemically. It lies within the capability of a skilled physician or veterinarian to select a form and route of administration that optimizes the effects of the compositions and treatments of the present invention to provide therapeutic effects, in to particular beneficial effects, more particularly sustained beneficial effects.

Hydrazide compounds and compositions may be administered in oral dosage forms such as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular forms, all utilizing dosage forms well known to those of ordinary skill in the pharmaceutical arts. Hydrazide compounds and compositions of the invention may be administered by intranasal route via topical use of suitable intranasal vehicles, or via a transdermal route, for example using conventional transdermal skin patches. A dosage protocol for administration using a transdermal delivery system may be continuous rather than intermittent throughout the dosage regimen. A sustained release formulation can also be used for the therapeutic agents.

In aspects of the invention the hydrazide compounds or compositions of the invention are administered by peripheral administration, in particular by intravenous administration, intraperitoneal administration, subcutaneous administration, intramuscular administration, oral administration, topical administration, transmucosal administration, or pulmonary administration.

A therapeutically effective dose of a hydrazide compound or composition of the invention for the treatment of a particular disorder or condition to provide effects, in particular beneficial effects, more particularly sustained beneficial effects, will depend on the nature of the disorder, and can be determined by standard clinical techniques. The precise dose to be employed in the formulation will also depend on the route of administration, and the seriousness of the disease or disorder, and should be decided according to the judgment of the practitioner and each patient's circumstances.

Suitable dosage ranges for administration are particularly selected to provide therapeutic effects, in particular beneficial effects, more particularly sustained beneficial effects. A dosage range is generally effective for triggering the desired biological responses. The dosage ranges for a hydrazide compound are generally about 0.01 mg to about 3 g per kg, 0.01 mg to about 2 g per kg, 0.5 mg to about 2 g per kg, about 1 mg to about 1 g per kg, about 1 mg to about 500 mg per kg, about 1 mg to about 200 mg per kg, about 1 mg to about 100 mg per kg, about 1 mg to about 50 mg per kg, or about 10 mg to about 100 mg per kg, of the weight of a subject, once, twice, or more per day, preferably once daily.

In aspects of the invention the dosages ranges are about 0.01 to 3000 mg/kg, 0.01 to 2000 mg/kg, 0.5 to 2000 mg/kg, about 0.5 to 1000 mg/kg, 0.1 to 1000 mg/kg, 0.1 to 500 mg/kg, 0.1 to 400 mg/kg, 0.1 to 300 mg/kg, 0.1 to 200 mg/kg, 0.1 to 100 mg/kg, 0.1 to 50 mg/kg, 0.1 to 20 mg/kg, 0.1 to 10 mg/kg, 0.1 to 6 mg/kg, 0.1 to 5 mg/kg, 0.1 to 3 mg/kg, 0.1 to 2 mg/kg, 0.1 to 1 mg/kg, 1 to 1000 mg/kg, 1 to 500 mg/kg, 1 to 400 mg/kg, 1 to 300 mg/kg, 1 to 200 mg/kg, 1 to 100 mg/kg, 1 to 50 mg/kg, 1 to 20 mg/kg, 1 to 10 mg/kg, 1 to 6 mg/kg, 1 to 5 mg/kg, or less than or about 10 mg/kg, 5 mg/kg, 2.5 mg/kg, 1 mg/kg, or 0.5 mg/kg twice daily or less.

In embodiments of the invention, the dosages ranges are about 0.1 to 1000 mg/kg, 0.1 to 500 mg/kg, 0.1 to 400 mg/kg, 0.1 to 300 mg/kg, 0.1 to 200 mg/kg, 0.1 to 100 mg/kg, 0.1 to 75 mg/kg, 0.1 to 50 mg/kg, 0.1 to 25 mg/kg, 0.1 to 20 mg/kg, 0.1 to 15 mg/kg, 0.1 to 10 mg/kg, 0.1 to 9 mg/kg, 0.1 to 8 mg/kg, 0.1 to 7 mg/kg, 0.1 to 6 mg/kg, or 0.1 to 5 mg/kg.

A composition or treatment of the invention may comprise a unit dosage of a hydrazide compound to provide beneficial effects, in particular one or more of the beneficial effects set out herein. A “unit dosage” or “dosage unit” refers to a unitary i.e., a single dose which is capable of being administered to a patient, and which may be readily handled and packed, remaining as a physically and chemically stable unit dose comprising the active agent as such or a mixture with one or more solid or liquid pharmaceutical excipients, carriers, or vehicles.

A hydrazide compound can be provided (preferably once daily or twice daily), in a single dosage unit or multiple dosage units (i.e., tablets or capsules) having about 10 to about 1000 mg, 10 to about 500 mg, 10 mg, 25 mg, 50 mg, 75 mg, 100 mg, 150 mg, 200 mg, 250 mg or 500 mg.

The dosage regimen of the invention will vary depending upon known factors such as the pharmacodynamic characteristics of the agents and their mode and route of administration; the species, age, sex, health, medical condition, and weight of the patient, the nature and extent of the symptoms, the kind of concurrent treatment, the frequency of treatment, the route of administration, the renal and hepatic function of the patient, and the desired effect.

Thus, a subject may be treated with a hydrazide compound or a composition of the invention on substantially any desired schedule. A hydrazide compound or composition of the invention may be administered one or more times per day, in particular 1 or 2 times per day, once per week, once a month, twice a month or continuously. However, a subject may be treated less frequently, such as every other day or once a week, or more frequently. A hydrazide compound or a composition of the invention may be administered to a subject for about or at least about 24 hours, 2 days, 3 days, 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, or 2 weeks to 24 months, periodically or continuously.

Hydrazide compounds, compositions and treatment methods described herein are indicated as therapeutic agents or methods either alone or in combination with other therapeutic agents or other forms of treatment. They may be combined or formulated with one or more therapies or agents used to treat a condition described herein. Compositions of the invention may be administered concurrently, separately, or sequentially with other therapeutic agents or therapies. Therefore, compounds of the formula I or II may be co-administered with one or more additional therapeutic agents for treating disorders disclosed herein as well as agents that are used for the treatment of complications resulting from or associated with a disease or disorder disclosed herein, or general medications that treat or prevent side effects. In embodiments of the invention, the additional therapeutic agent is a vacuolar-type (H+)-ATPase-inhibiting compound (e.g., a concanamycin, a bafilomycin, a benzolactone enamide, such as a salicylihalamide or a lobatamide, enoxacin, and doxycycline). One or more additional anti-resorptive compounds or anticancer compounds, other than a compound of the present invention, also can be included. The additional therapeutic agent can be a bone enhancing agent such as a synthetic hormone, a natural hormone, oestrogen, calcitonin, tamoxifen, a biphosphonate, a biphosphonate analog, vitamin D, a vitamin D analog, a mineral supplement, a statin drug, a selective oestrogen receptor modulator or sodium fluoride.

The compounds of the present invention can be utilized in a variety of therapeutic and non-therapeutic applications. It will be appreciated that one or more compounds of the present invention can be used, for example, as a control in diagnostic kits, bioassays, or the like. Preferably, the method of the present invention is applied therapeutically, for example, toward the treatment or prevention of a disease or disorder contemplated herein, (e.g. osteoporosis, cancer) or toward the treatment or prevention of a condition (e.g., an abnormal condition or a disease) treatable by the inhibition of vacuolar-type (H+)-ATPase.

### Assay Systems

The present disclosure contemplates materials and methods for detecting and inhibiting the interaction of V-ATPase subunit proteins, preferably the a3 and B2 subunit isoforms. In an aspect the V-ATPase polypeptides are of mammalian origin. In an exemplified embodiment the disclosure describes materials and methods for detecting the interaction of the murine V-ATPase subunit isoform proteins a3 and B2. A method of the present disclosure for detecting or determining the interaction of a first V-ATPase polypeptide with a second V-ATPase polypeptide comprises contacting the V-ATPase polypeptides and determining whether the polypeptides interact, using a system where if interaction does occur then a detectable signal or change is induced in the assay system.

In one embodiment, the method is used to determine the extent to which a first V-ATPase subunit polypeptide (e.g. a3) interacts with a second V-ATPase subunit polypeptide (e.g. B2) either in their native form or as fragments thereof, or with various modifications to either subunit protein, such as amino acid or polypeptide substitutions, additions, deletions, mutations, chemical modification, etc. The subject disclosure also concerns materials and methods for screening for drugs or compositions that inhibit, or modify the degree of interaction of V-ATPase subunit proteins. In one embodiment, the assay is used to screen a large number of compounds in a high throughput format with the ultimate aim of discovering drugs or compositions for reducing or preventing the interaction of V-ATPase subunits. In one embodiment, drugs or compositions so discovered are used to inhibit V-ATPase-mediated acid secretion of mammalian cells.

In an aspect, the disclosure provides a method for detecting or determining the interaction of V-ATPase subunit polypeptides comprising:

(a) providing a first V-ATPase subunit polypeptide, or portion thereof;

(b) providing a second V-ATPase subunit polypeptide or portion thereof, and

(c) incorporating said first V-ATPase subunit polypeptide, either in solution or soluble suspension into a vessel, or immobilized passively or covalently onto a suitable carrier, and exposing the first polypeptide to the second V-ATPase subunit polypeptide, in solution or soluble suspension, to determine if the second polypeptide interacts with the first polypeptide, and

(d) detecting and quantifying the interaction of the polypeptides.

The subunit polypeptides may be natural or genetically engineered polypeptides and may additionally comprise amino-terminal or carboxy-terminal fusion protein(s) or epitope tag polypeptide(s), or internal epitope tag polypeptide(s). Examples of fusion protein partners are glutathione-5-transferase and thioredoxin. Examples of epitope tags are hexahistidine (His₆, H₆) and diglycinylhexahistidinyldiglycine (Gly₂His₆Gly₂, G₂H₆G₂).

Examples of suitable carriers include, but are not limited to, glass, quartz or plastic vessels, glass, synthetic polymers, biopolymer beads, filters, gold, silicon, plasmon-bearing or semiconductor surfaces on microchips, or resonant quartz crystals.

In step (d) the interaction may be detected and quantified using biochemical or physical methods known in the art, for example, by using methods including, but not restricted to, enzyme linked immunosorbent assay (ELISA), surface plasmon resonance (SPR), nuclear magnetic resonance (NMR), mass spectrometry (MS), analytical ultracentrifugation (AUC), or dynamic light scattering (DLS).

Two hybrid systems which may be employed herein are described for example in U.S. Pat. Nos. 5,283,173 and 5,468,614. Two hybrid reagents are also available from commercial suppliers such as Clontech Laboratories (Palo Alto, Calif.) and Stratagene (La Jolla, Calif.). In an embodiment, all or a portion of a first mammalian V-ATPase gene is cloned into a suitable plasmid that, when expressed in a host cell, provides a hybrid protein comprising a first V-ATPase polypeptide, or a variant or fragment thereof, and a DNA-binding domain of a transcriptional activator that can bind to a site on a detectable gene in the host cell. All or a portion of a second V-ATPase gene is also cloned into a suitable plasmid that, when expressed in the host cell, provides a hybrid protein comprising a second V-ATPase polypeptide, or a variant or fragment thereof, and a transcriptional activation domain that can activate transcription of the detectable gene in the host cell when the transcriptional activation domain is brought into close proximity with the detectable gene. Preferably, the first and second V-ATPase polypeptides are the a3 and B2 subunits; however, the use of first and second V-ATPase subunit polypeptides that are different, e.g. in amino acid sequence or length, can also be employed in the method. The sequences of the DNA and polypeptide amino acids coding for the human V-ATPase a3 subunit are described in U.S. Pat. Nos. 6,403,304 and 6,777,537. The full length sequences of murine a3 cDNA and polypeptide are shown in SEQ ID NO:1 and SEQ ID NO:2, respectively. The full length sequences of murine B2 cDNA and polypeptide are shown in SEQ ID NO:3 and SEQ ID NO:4, respectively. All V-ATPases are highly similar throughout eukaryotic organisms and there is a high degree of homology between the Mus musculus and Homo sapiens V-ATPase subunits. The expression plasmids encoding the hybrid proteins can be introduced into a host cell using standard methods known in the art, such as the many commercially available transfection agents. Preferably, the portion of the V-ATPase a3 subunit expressed in the hybrid proteins includes the amino-terminal domain, NTa3 (amino acids 1-393) or a functional fragment thereof. The polynucleotides encoding the first and second fusion proteins are exposed to conditions where the fusion proteins are expressed. If the V-ATPase polypeptides of the expressed hybrid proteins interact, then the DNA binding domain and transcriptional activation domain of the transcriptional activator are brought into close proximity, sufficient to cause transcription of the detectable gene. Where transcription or expression of the detectable gene is observed, this is indicative of interaction of the V-ATPase portion of the fusion proteins.

In one exemplified embodiment, a3 and B2 subunit interactions are detected using a yeast two hybrid system. This is described in Example 1, which details the construction of a yeast two hybrid system to discover interactions of NTa3 with other proteins expressed in RANKL-differentiated osteoclast-like RAW 264.7 cells. In this example the interaction of a3 and B2 is discovered and is shown to be significant, using assays that detect the expression of the independent reporter genes, HIS3 and lacZ. In another exemplified embodiment, the ability of ‘a’ subunits of all isoforms (a1-a4) to pull down B subunits of all isoforms (B1 and B2) when both proteins are purified, and vice versa, is demonstrated. By “pull down” it is meant that a first V-ATPase subunit is immobilized by adsorption to beads that have affinity for its fusion partner, and the beads are then used to adsorb a second V-ATPase subunit that is in solution, by virtue of the affinity for interaction between the first and second V-ATPase subunits. This occurrence can be demonstrated by gel electrophoresis and immunoblotting methods that are well known in the art, and by using appropriate controls to eliminate artefacts, as would be apparent to one skilled in the art. The data shown further verify the interaction of ‘a’ and B subunits, and especially a3 and B2 subunit isoforms.

The V-ATPase a3 and B2 subunits interact in such a manner as can be quantified by a number of assays known to the art for the determination of the extent of protein interactions. In another exemplified embodiment, the a3 and B2 subunit interactions are detected using an ELISA system. ELISA is a method that can quantify binding interactions and elucidate saturation binding curves. The interaction of the immobilized first polypeptide and the soluble second polypeptide in the ELISA system causes a measurably greater detection of the second polypeptide than that observed where the first polypeptide and the second polypeptide do not interact or interact at a reduced level. The detectable interaction can be between any portion of either the a3 or B2 protein that retains the capability of detectable interaction. Typically, a3-B2 interaction is detected directly, by means of a physical tag, e.g. a fluorophore, or indirectly, e.g. by the action of an enzyme tag on a suitable chromogenic substrate. Suitable detection methods are well known in the art.

Methods for detecting protein interactions mediated by small molecule ligands have been described in the art (Berlin, 1997). In one exemplified embodiment, the detection method is as described in Example 1. Here the amino-terminal domain of murine a3, NTa3 (containing amino acids 1-393) and the full length murine B2 (amino acids 1-511) were cloned into the pET32 and pGEX vectors, respectively, to produce TRX and GST fusion proteins by expression in E. coli. These proteins were purified by affinity chromatography on nickel (II) or glutathione Sepharose 4B beads, respectively. Purified fusion proteins were used in the ELISA method by coating commercial ELISA plates with TRX-NTa3 (the ligand) and probing with soluble GST-B2 (the analyte). Detection of bound analyte was performed by probing with an antibody directed against GST, followed by a second enzyme-conjugated antibody directed against the first antibody. The second antibody amount, which is proportional to the amount of analyte bound to the plate, was determined by a color reaction using a chromogenic compound that is converted to a colored compound by the action of the enzyme conjugated to the second antibody. Data derived from ELISA analysis can be used to compare relative affinities of binding interactions. This is shown in Example 1, which details the methods of the ELISA system. The data shown again verify the interaction of the a3 and B2 subunits and further delineate that the binding is between the N-terminal domain of a3 and the C-terminal domain of B2.

In additional contemplated embodiments, the interaction of a first V-ATPase polypeptide with a second V-ATPase polypeptide can be detected in a host cell by the interaction of signal transduction fusion proteins, or by the interaction of proteins resulting in cleavage of a ubiquitin fusion protein. These methods of detecting protein-protein interactions, by SOS recruitment (Aronheim et al., 1997) or by a split ubiquitin sensor (Johnsson and Varshaysky, 1997), respectively, are well known to those skilled in the art. The preferred host cell for these embodiments is yeast. In another embodiment, the interaction of a first V-ATPase polypeptide with a second V-ATPase polypeptide is detected by interaction of signal transduction fusion proteins within a bacterial cell. Methods for detecting protein-protein interactions in bacteria and mammalian cells are also known by those skilled in the art (Karimova et al., 1998).

A V-ATPase portion of a fusion protein can contain one or more mutations of the wildtype amino acid sequence. The mutations contemplated can include amino acid substitutions, deletions and insertions. Any mutation, including e.g. mutations to V-ATPase already known in the art and associated with osteopetrosis, can be prepared in the sequence of the V-ATPase polypeptide and used in the methods of the present disclosure. The V-ATPase portion of the fusion protein can include the entire coding sequence of the protein or a fragment thereof. The V-ATPase portion of the fusion protein can include sequences from widely divergent species, since V-ATPase subunit protein sequences are highly conserved. In an exemplified embodiment, the V-ATPase subunit protein sequences are derived from the mouse, Mus musculus (M. musculus).

Many methods for detection of a bound second fusion protein are available and are well known in the art. Biochemical or physical methods other than ELISA, as described herein, including but not restricted to surface plasmon resonance (SPR), nuclear magnetic resonance (NMR), mass spectrometry (MS), analytical ultracentrifugation (AUC), or dynamic light scattering (DLS) are contemplated herein.

The subject disclosure also concerns materials and methods for screening compositions and drugs in a high throughput format to identify and select compounds that inhibit or prevent the interaction of V-ATPase subunit interactions. The present invention encompasses compounds, drugs or compositions discovered by utilizing the assay methods disclosed herein.

In an exemplified embodiment, an ELISA system is used to discover inhibitors of V-ATPase subunit a3 and B2 interaction. Thus, a method is disclosed for detecting a modulator (e.g. an inhibitor) of the interaction of a first V-ATPase polypeptide with a second V-ATPase polypeptide by (a) providing a first fusion protein comprising all or a portion of a first V-ATPase polypeptide which is immobilized (e.g., by passive adsorption in a well of a multi-well plastic dish) and (b) providing a solution containing a suitable concentration of a composition or drug from a group of test compounds and (c) providing a second fusion protein comprising all or a portion of a second V-ATPase polypeptide that is in solution, mixed with the test compound, and (d) contacting the first fusion protein and the second fusion protein in the presence of the test compound under conditions where if the first fusion protein and the second fusion protein interact then they form a stable complex which can be separated from unbound second fusion protein and (e) detecting the bound second fusion protein by immunochemical agents directed towards the fusion-partner moiety of the second fusion protein.

“Detecting” refers to determining the amount of second fusion protein bound to the immobilized first fusion protein using quantitative or semi-quantitative methods known in the art. In an embodiment, detection is accomplished by the additional binding of an antibody, specifically directed toward the fusion-partner moiety of the second fusion protein. The antibody is conjugated with an enzyme and can be quantified using a chromogenic substrate followed by spectrophotometry, the amount of color produced being proportional to the amount of the second V-ATPase polypeptide bound in the assay well. Reduction in amount of detectable second V-ATPase polypeptide bound, compared with controls where only vehicle is added to the well instead of a test compound, indicates that the test compound inhibits the interaction between the first and second V-ATPase polypeptides.

The first and second fusion proteins required to perform the ELISA system in a high-throughput fashion may be prepared by engineering DNA sequences that code for the fusion protein for insertion into an expression vector, such as a plasmid or a virus. Protein expression is performed by transformation or infection of suitable host cells, followed by growth and induction of expression, by methods well known in the art. The host cells for expression of a3, B2, or fragments thereof can be any suitable prokaryotic or eukaryotic cell, including bacterial, yeast, insect or mammalian cells. In a preferred embodiment, the host cell is a bacterial cell. More preferably, the bacterial cell is Escherichia coli (E. coli).

In an embodiment the disclosure provides a method of identifying a drug or composition that modulates (e.g. reduces, nullifies of inhibits) interaction of V-ATPase subunit polypeptides comprising:

(a) introducing in a protein-protein interaction method disclosed herein a natural or synthetic test compound prior to and/or during the exposure of the first V-ATPase subunit polypeptide to the second V-ATPase subunit polypeptide, and

(b) quantifying the reduction in the degree of interaction between the first V-ATPase subunit polypeptide and second V-ATPase subunit polypeptide in the presence of said test compound, and comparing with the degree of interaction in the absence of said test compound, thereby determining whether the said test compound modulates the interaction of V-ATPase subunit polypeptides.

In embodiments of assay methods disclosed herein, the first V-ATPase subunit polypeptide, or portion thereof is the amino-terminal domain of the mouse (Mus musculus, M. musculus) Atp6i (a.k.a. Tcirg1, Atp6v0a3, etc.) gene product, namely the V-ATPase V₀ polypeptide subunit a, isoform 3 (NTa3; amino acids 1-393, with respect to the initial methionine of the natural gene product, and with reference to the National Center for Biotechnology Information (NCBI) reference sequence collection accession no. NM_{—}016921.2 (the full length amino acid sequence of which is defined in SEQ ID NaI)).

In embodiments of assay methods disclosed herein, the second V-ATPase subunit polypeptide is the full-length M. musculus Atp6v1b2 gene product, namely the V-ATPase V₁ polypeptide subunit B, isoform 2 (B2; amino acids 1-511, with respect to the initial methionine of the natural gene product, and with reference to the NCBI reference sequence collection accession no. NM_{—}007509 (the full length amino acid sequence of which is defined in SEQ ID NO:2)).

In embodiments of assay methods disclosed herein, the V-ATPase subunit polypeptides are variants that are not of M. musculus origin, or are chimaeras or mutations, having greater than 30%, 40%, 50%, 60%, 70%, 80%, 90% or 95% homology to the polypeptides of SEQ 1N NO 1 or 2. In an assay method disclosed herein the V-ATPase subunit polypeptides comprise truncations of natural or variant V-ATPase polypeptides. In an assay method disclosed herein an expression host is used to produce the first and second polypeptides. In embodiments, the host is E. coli.

A screening method generally comprises performing a series of controls to confirm that the inhibition of the interaction of the first V-ATPase subunit polypeptide (e.g., NTa3) and second V-ATPase subunit polypeptide (e.g., B2) by the test compound is specific to the interaction (e.g., a3-B2 interaction). For example, the method may comprise confirming that the test compound does not appreciably inhibit test cell growth, or a1-B2, a2-B2, or a4-B2 V-ATPase subunit interactions, where a1, a2 and a4 are isoforms 1, 2 and 4 of the V-ATPase ‘a’ subunit, respectively, with interactions of subunits being indicated here by a hyphen).

Test compounds include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], and small organic or inorganic molecules. The agents or compounds may be endogenous physiological compounds or natural or synthetic compounds.

A test compound may be selected from the group consisting of a naturally occurring organic chemical compound, or a synthetic organic chemical compound, or a polypeptide or a biologically active fragment thereof, or a non-peptidyl-backbone peptide mimic (peptidomimetic), or an antibody or antigen-binding fragment thereof, or a polynucleotide or polynucleotide mimetic, or a polynucleotide-polypeptide hybrid.

A method disclosed herein may be used to select a lead candidate for further development as a potential therapeutic agent in the treatment of a disease or condition disclosed herein such as pathological bone loss, which includes but is not restricted to osteoporosis, inflammatory arthritis, periodontal disease and metastatic bone cancer in human medicine and veterinary medicine, and as a treatment to prevent invasion and metastasis in cancer in human medicine and veterinary medicine. In embodiments a lead compound is selected based on its ability to cause significant reduction in the degree of interaction between the subunit polypeptides. In embodiments, the lead compounds are drugs, synthetic chemical compounds, naturally occurring chemical compounds, polypeptides and biologically active fragments thereof, antibodies or antigen-binding fragments thereof, polynucleotides and other agents identified using the disclosed methods that inhibit a3-B2 interaction.

A lead compound may be tested for its ability to inhibit the interaction of V-ATPase subunit polypeptides a3 and B2 using methods disclosed herein. In particular, a lead compound may be contacted with target cells such as RANKL-differentiated RAW 264.7 mouse osteoclast-like tissue culture cells or mammalian osteoclasts derived from precursor cells in bone marrow or spleen, differentiated with M-CSF and RANKL. Preferably the target cells are mammalian osteoclasts within the living organism of their origin.

Further to their detectable inhibition of a3 and B2 subunit interactions in an ELISA system, lead compounds can then be secondarily tested for activity to inhibit V-ATPase function, and consequently acid secretion, in mammalian cells. Drugs or compositions that inhibit V-ATPase function in vitro can be further evaluated to confirm in vivo efficacy in treating a disease or disorder disclosed herein (e.g. clinical bone loss disease or preventing tumor metastasis). Thus, the subject disclosure also concerns materials and methods for identifying drugs or compositions useful in treating bone loss pathology and cancer.

The invention will be described in greater detail by way of specific examples. The following examples are offered for illustrative purposes, and are not intended to limit the invention in any manner.

### Example 1

The work presented here approaches the problem of further characterizing V-ATPase structure and function by implementing yeast two hybrid studies using a cDNA library derived from the murine RAW 264.7 cell line, which is capable of differentiating into osteoclasts in the presence of RANKL {Raschke, 1978; Hsu, 1999; Collin-Osdoby, 2003}. This system is of particular interest because of the high level of expression of V-ATPase, which is responsible for the proton secretion into the extracellular lacunae of osteoclasts through their specialized ruffled border. This function is required to dissolve bone {Blair, 1989; Vaananen, 1990}, and disruption results in the sclerosing bone disorder, osteopetrosis {Del Fattore, 2008; Kane, 2007}, while excessive activity results in pathological bone loss, as in osteoporosis. Further understanding of the structure and function of V-ATPases will aid the development of targeted therapeutics for the treatment of bone loss diseases, such as osteoporosis. {Xu, 2007}

### Experimental Procedures

Materials and reagents—PBS was 0.2 g/l KCl, 0.2 g/l KH₂PO₄, 8.0 g/l NaCl, 1.15 g/l Na₂HPO₄, pH 7.4. TBST was 20 mM Tris-HCl, 0.9% (w/v) NaCl, 0.05% (w/v) Triton X-100, pH 7.40 at 25° C. Gelatin for blocking in pulldown assays and ELISA was EIA grade (Bio-Rad, cat. no. 170-6537). Glutathione Sepharose 4B was from GE Healthcare (Baie d'Urfe, Quebec; cat. no. 17-0756-01). Ni-NTA agarose was from Qiagen (Mississauga, Ontario; cat. no. 30210). Anti-GST antibody was rabbit polyclonal GST(Z-5), from Santa Cruz Biotechnology (Santa Cruz, Calif.; cat. no. sc-459). Horseradish peroxidase-conjugated second antibody (HRP-GAR) was polyclonal goat anti-rabbit IgG from Santa Cruz (cat. no. sc-2004).
Cell culture—The S. cerevisiae strain YRG-2 (Stratagene) was maintained on YPAD agar plates, as recommended by the supplier. Transformed yeast in two hybrid experiments were grown on SD agar plates, or in SD liquid media, with amino acid dropouts appropriate to the experiment, according to supplier formulations and recommendations. Cloning and plasmid production were done in the E. coli strain DH5α (a common lab strain), pET vector protein expression in the strain BL21(DE3) Rosetta (Novagen, a division of EMD Chemicals, Gibbstown, N.J.), and pGEX vector protein expression in the strain K12 KS1000 (New England Biolabs, Pickering, Ontario). Murine macrophage RAW 264.7 cells (American Type Culture Collection (ATCC), cat. no. TIB-71) were grown in Dulbecco's modified Eagle's medium (DMEM) with high glucose (4.5 g/l), L-glutamine, sodium bicarbonate, and without sodium pyruvate (Sigma-Aldrich, St. Louis, Mo.; cat. no. D5796), supplemented with 100 u/ml penicillin, 100 μg/ml streptomycin and 10% (v/v) fetal bovine serum (Invitrogen Canada, Burlington, Ontario; cat. no. 10437-028). Differentiation of RAW 264.7 cells to osteoclasts was performed by exposure to 100 ng/ml soluble recombinant RANKL in medium for three days. This resulted in a mix of cells, containing 1 to 20 nuclei, >90% positive for tartrate-resistant acid phosphatase (TRAP) staining.
Yeast two hybrid assays—The commercial HybriZAP 2.1 system (Stratagene, cat. nos. 235612 and 235614) was used throughout. Initial screening was done with a cDNA library constructed according to supplier recommendation (HybriZAP-2.1 XR Library Construction Kit and HybriZAP-2.1 XR cDNA Synthesis Kit, Instruction Manual, Revision B.01), using oligo-dT magnetic bead-purified mRNA (Absolutely mRNA purification kit, Stratagene; cat. no. 400806) from RANKL-differentiated RAW 264.7 cells (described above). After reverse transcription of mRNA, second strand synthesis, ligation of linkers, and restriction enzyme digestion (as per suppliers instructions), size-selected cDNA was inserted between EcoRI and XhoI sites in the HybriZAP 2.1 lambda phage vector. The primary lambda phage library was mass excised to yield a pAD-GAL4-2.1 cDNA phagemid library. The library was screened in YRG-2 host yeast (genotype Mat .alpha., ura3-52, his3-200, ade2-101, lys2-801, trpl-901, leu2-3, 112, gal4-542, gal80-538, LYS2::UAS.sub.GAL1-TATA .sub.GAL1-HIS3 URA::UAS.sub.GAL4-17 mers(×3) TATA .sub.CYC1-lacZ; Stratagene) with a bait construct comprising the N-terminal, cytoplasmic domain of the murine V-ATPase subunit a3 (a.a. 1-393) inserted between EcoRI and SalI sites in the pBD-GAL4 Cam phagemid vector. Additionally, a 5′-ggtggt-3′ linker (encoding Gly-Gly) was constructed in frame between the EcoRI site and the a3 subunit start codon. Sequences for a3 were obtained by PCR using the a3-containing vector pcDNA3.1-a3 as template, a gift of Dr. Beth S. Lee (Dept. of Physiology and Cell Biology, Ohio State University).
Constructs—Full length mouse B1 subunit was cloned by PCR using as template the B1-containing vector pEF6/V5-His-TOPO-B1, a gift from Dr. Beth S. Lee (Dept. of Physiology and Cell Biology, Ohio State University). The oligonucleotide primers used are described in Table 2. The PCR product was agarose gel purified (GFX PCR DNA and gel band purification kit; GE Healthcare), digested with EcoRI and SalI and cloned into EcoRI/SalI digested pAD-GAL4 2.1 (Stratagene). To facilitate further cloning, the internal EcoRI site at nt. 319-324 (w.r.t. B1 ORF sequence) was knocked out with the silent mutation a321 g (QuickChange site-directed mutagenesis kit; Stratagene). Constructs for other V-ATPase subunits followed the same strategy, but PCR templates were from varied sources, and inserts were cloned into EcoRI/SalI sites in pBD GAL4 Cam, pET-32a(+) (Novagen), or pGEX-4T-1 (GE Healthcare), as elaborated in Table 2. All expression clones contained sequences for a Gly-Gly flexible coupler (G₂) between their N-terminus and the C-terminus of the fusion protein partner (either thioredoxin (TRX) or glutathione-S-transferase (GST), respectively). As noted, full length B1 and B2 constructs also had a C-terminal His-tag, 6×His with flanking Gly-Gly (G₂H₆G₂), to facilitate full-length purification. Clones that were made by RT-PCR from total RNA extracts of mouse tissues were done by extraction with TRIzol Reagent (Invitrogen) and reverse transcription using the RevertAid H minus first strand cDNA synthesis kit (Fermentas; cat. no. K1632).
Purification of GST fusion proteins—Bacteria transfected with expression constructs that were growing exponentially at 37° C., in LB medium with selective antibiotics, were chilled on ice and IPTG was added to 0.2 mM. Cells were incubated further with shaking at 16° C. for 16 h. Cells were harvested by centrifugation at 4° C. and pellets were resuspended in 4 ml ice cold PBS, containing 0.2 mg/ml lysozyme per 200 ml of original culture volume. The suspension was incubated on ice for 30 min. then mixed with 2.5 volumes of ice cold 0.2% v/v Triton X-100. This was sonicated on ice for 4×15 seconds with 30 seconds cooling between bursts. DNase and RNase were added to 5 μg/ml each, from 10 mg/ml stocks, and the lysate was incubated a further 10 min. on ice then centrifuged at 20,000 g for 15 min. The supernatant was mixed with 10 ml, per liter of starting culture, with a 50% slurry of Glutathione Sepharose 4B in PBS. After 1 h incubation at 4° C. with rocking, beads were washed 3×5 min. with PBS and collected by centrifugation at 500 g for 5 mM. The beads were transferred to a Poly-Prep chromatography column (Bio-Rad) and washed with 2×20 ml PBS and eluted with 10 mM reduced glutathione in PBS. Fractions were collected and analyzed by SDS-PAGE. GST-B1(GH) and GST-B2(GH) were repurified on to Ni-NTA agarose beads, as described for the TRX fusion protein purification.
Purification of TRX fusion proteins—This procedure was similar to purification of GST fusion proteins with the following exceptions. Bacterial pellets were resuspended in ice cold lysis buffer consisting of 50 mM NaH₂PO₄, 300 mM NaCl and 10 mM imidazole, pH 8.0 at 25° C. at a ratio of 4 ml per 60 ml of original culture volume. After lysis, cleared supernatants were mixed with 2 ml of a 50% slurry of Ni-NTA agarose beads in lysis buffer at 4° C. for 1 h with rocking. Beads were washed 3×5 min. with a 10 ml wash buffer containing 50 mM NaH₂PO₄, 300 mM NaCl and 20 mM imidazole, pH8.0 and packed in a Poly-Prep chromatography column. The column was washed twice with 20 ml of wash buffer and eluted with 50 mM sodium phosphate, 300 mM NaCl, 50 mM imidazole, pH 8.0. A second elution was performed at 150 mM imidazole conc. Fractions were analyzed by SDS-PAGE.
Pulldown assays—Glutathione Sepharose 4B beads (0.5 ml packed volume) were combined with 4 ml of cleared bacterial lysate containing 0.35 mg/ml total protein and PMSF to 2 mM (from 0.2 M stock in anhydrous ethanol), from either GST-B1 or GST-B2 expression cultures. After coating at 4° C. for 1 hour with rocking, the beads were washed 5 times with 10 ml PBS and 125 μl of coated beads was mixed with 400 μl of cleared supernatants of TRX-NTa1-4 expression cultures containing 0.13 mg/ml protein. After a 1 h incubation at 4° C. with rocking, beads were washed 5 times with 1 ml ice cold PBS. Pelleted beads were eluted with 100 μl of SDS sample buffer and 10 μl aliquots were analyzed by SDS-PAGE.
Blot overlays—Bacteria growing exponentially in selective media (1.5 ml culture) were induced with 0.2 mM IPTG for 3 h at 25° C., then pelleted and dissolved in 50 μl of 1 M NaOH. The solution was immediately neutralized with 50 μl of 1 M acetic acid, and 1 μl each of RNase and DNase stock (10 mg/ml) was added. After 10 min. incubation at 4° C., SDS sample buffer was added and samples were heated at 100° C. for 5 min. Samples were microfuged 10 min. at 14,000 g and clear supernatants containing 5 μg protein were run on 8% SDS-PAGE and transferred to nitrocellulose. Blots were blocked with 1% gelatin in TBST and overlaid with 1 μg/ml purified GST-B2(GH), 16 h at 4° C. The blot was washed 3×5 min. in TBST, then probed with anti-GST antibody (1:2500), 2 h at 25° C., washed, then probed with HRP-GAR (1:2500), washed, and developed using enhanced chemiluminescence substrate (Western Lightning Plus, Perkin Elmer) and imaged using a Molecular Imager Gel Doc XR+ system (Bio-Rad).
ELISA solutions—Protein Coating buffer was 10 mM sodium phosphate, pH 7.0; Tris-buffered saline (TBS) was 20 mM Tris-HCl, 0.9% (w/v) NaCl, pH 7.40 at 25° C.; Blocking Buffer was 1% w/v gelatin in TBS containing 0.05% (w/v) Triton X-100 and 0.1% (w/v) phenol, pH 7.40; Wash Buffer was TBST; TMB stock was 4 mg/ml 3,3′,5,5′-tetramethybenzidine (Sigma-Aldrich) in dimethylsulfoxide (DMSO)/ethanol (1:9), Developer was 40 μg/ml TMB (from stock), 0.01% hydrogen peroxide, 0.1 M sodium acetate, pH 6.0; Stop Solution was 1 N sulfuric acid; anti-GST antibody was a 1:4000 dilution of GST(Z-5) stock in Blocking Buffer; HRP-GAR was a 1:4000 dilution from stock in Blocking Buffer.
In vitro ELISA-based binding assays—Volumes are per well. All washes were done with 100 μl Wash Buffer (see ELISA solutions). High protein-binding polystyrene 384-well plates (Greiner Microlon 781097; Sigma-Aldrich cat. no. M6936) were coated with ligand protein (20 μl Protein Coating Buffer containing 10 μg/ml TRX-NTa3), overnight at 4°. All further steps were at 25° C. Coated plates were washed twice, blocked with 30 μl Blocking Buffer for 1 h, and again washed twice. For binding experiments, wells were then incubated with analyte protein (20 μl Blocking Buffer with 5 mM MgCl₂, containing GST-B2(GH), concentrations as described), for 1 h. Plates were washed three times, followed by incubation with 20 μl anti-GST antibody in Blocking Buffer for 30 min. Plates were again washed three times and incubated with 20 μl of HRP-GAR in Blocking Buffer for 30 min. Plates were then washed five times, followed by addition of 30 μl of Developer. After 15 min. of incubation, the reaction was stopped with 30 μl of Stop Solution. Absorbance was quantified using a Perkin Elmer Envision Multilabel Reader at 450 nm, with subtraction of a reference absorbance at 600 nm.

The results are illustrated and discussed below and in the Figures.

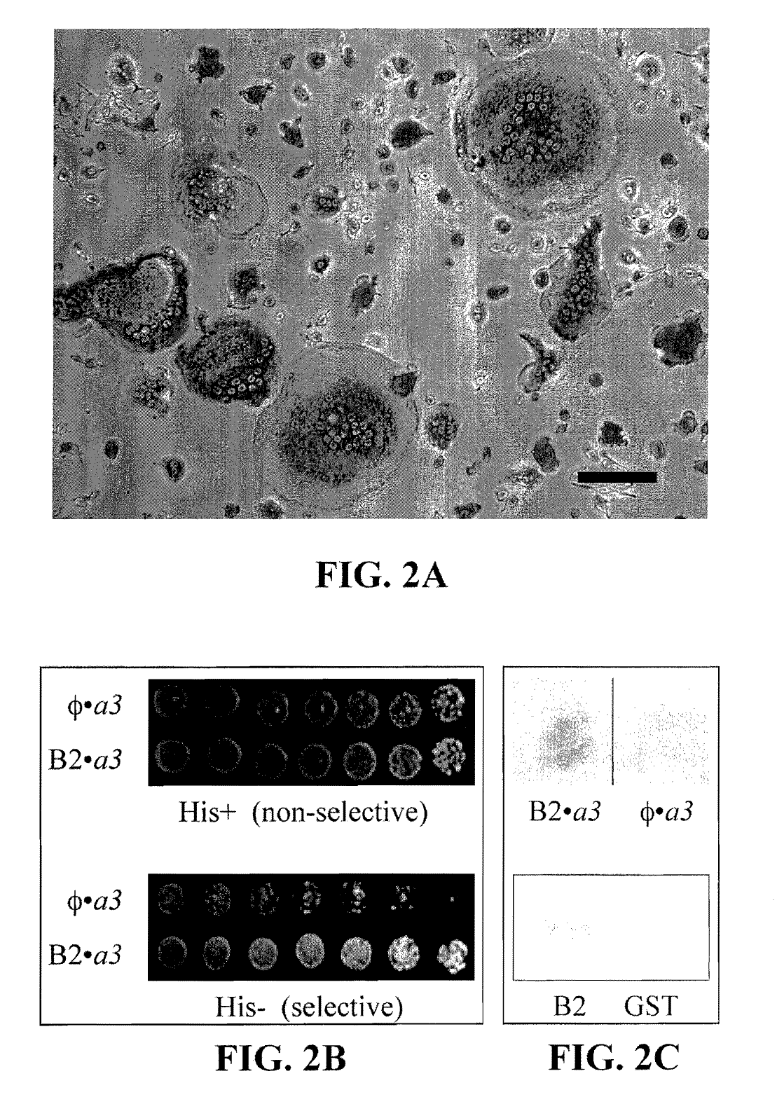

The initial intention of this work was to determine the set of protein binding partners that interact specifically with the a3 subunit that is highly expressed as part of the plasma membrane V-ATPase of osteoclasts. Crucial interactions with it could potentially be exploited as molecular targets for the treatment of bone-loss disease. Therefore, a mouse cDNA library was constructed from RANKL-differentiated RAW264.7 cells, representing a range of osteoclasts from single TRAP-positive cells to cells containing over 20 nuclei (FIG. 2A), in the HybriZap 2.1 phagemid vector system (Stratagene). The cDNA library was probed in YRG-2 yeast using a construct of NTa3 (the N-terminal domain of a3, a.a. 1-393; see Table 2) in the yeast two-hybrid GAL4 binding domain vector pBD GAL4 Cam (Stratagene). The outcome of this screening was difficult to interpret because of unresolved issues with a high background of self-activation with all a subunit constructs attempted; however, a strong interaction that was consistently above background was found with the full-length B2 subunit of V-ATPase. This interaction was confirmed using the second reporter gene, lacZ, and by affinity pulldowns of NTa3 with B2-coated glutathione-Sepharose 4B beads (Amersham) (FIG. 2B).

Further confirmation of in interaction was sought by purifying proteins and observing their interactions in a cell free system. To obtain purified protein, constructs were made in bacterial expression vectors. B1 and B2 subunit isoform sequences were cloned into pGEX-4T-1 to express the subunits as fusions with glutathione-S-transferase (GST) and N-terminal domains a1-a4 subunits were cloned into pET32a(+) to express the subunit domains as fusions with thioredoxin (TRX). The construction of these expression plasmids is described in Table 2. Henceforth, whenever interactions between ‘a’ and ‘B’ subunits are referred to, as e.g. the interaction between a3 and B2, it is to be understood that the interaction is inferred from experimental interactions between fusion proteins; namely, B subunit isoforms with amino-terminal GST fusions, and N-terminal domains of ‘a’ subunits (NTa) with N-terminal TRX fusions. Appropriate control experiments demonstrate that subunit interaction referred to herein are between the V-ATPase subunit polypeptides and not the fusion partners, GST and TRX. Purified proteins were prepared as described under Materials and Methods, but can be obtained by many methods that would be immediately apparent to those skilled in the art and are contemplated within the scope of the invention. Subsequently, a second method of confirmation of the interaction between NTa3 and B2 was performed using affinity pulldowns of NTa3 with B2-coated glutathione-Sepharose 4B beads (see below).

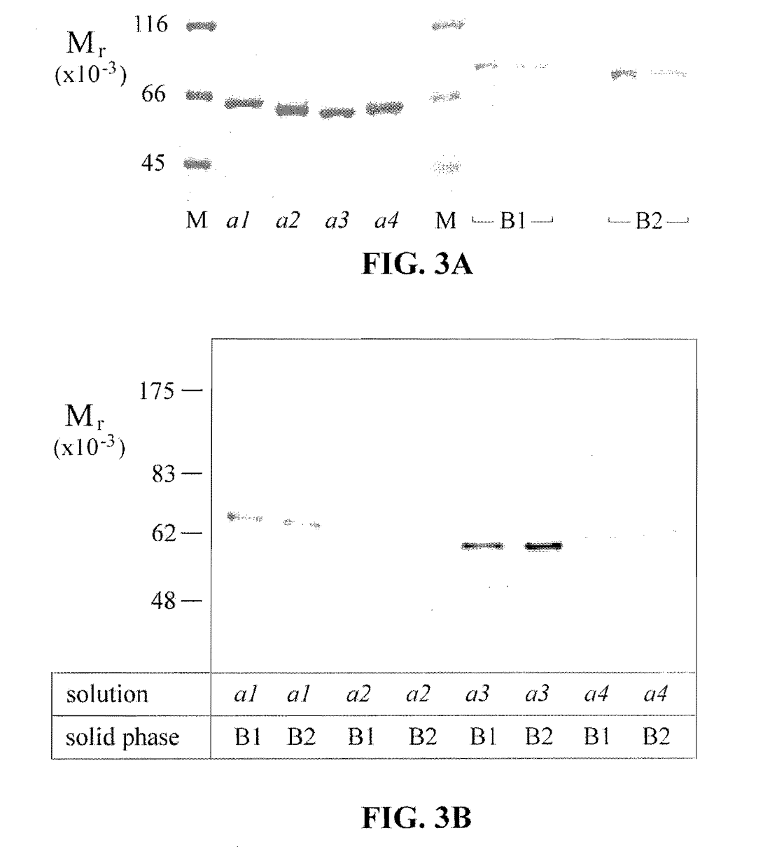

It was of interest to determine whether there were differences among the four a subunits isoforms (a1-4) in terms of their interactions with the two β isoforms (B1 and B2). TRX-fusion proteins, NTa1, NTa2, NTa3 and NTa4 were expressed and purified (FIG. 3A; see also Experimental Procedures). Affinity pulldowns, as shown in FIG. 3B, were performed, showing that a1, a3 and a4 were efficiently pulled down with both B1 and B2, with somewhat less apparent affinity for a2; however, differences between the abilities of B1 and B2 to pull down the four isoforms of the NTa domain seemed negligible in replicate experiments.

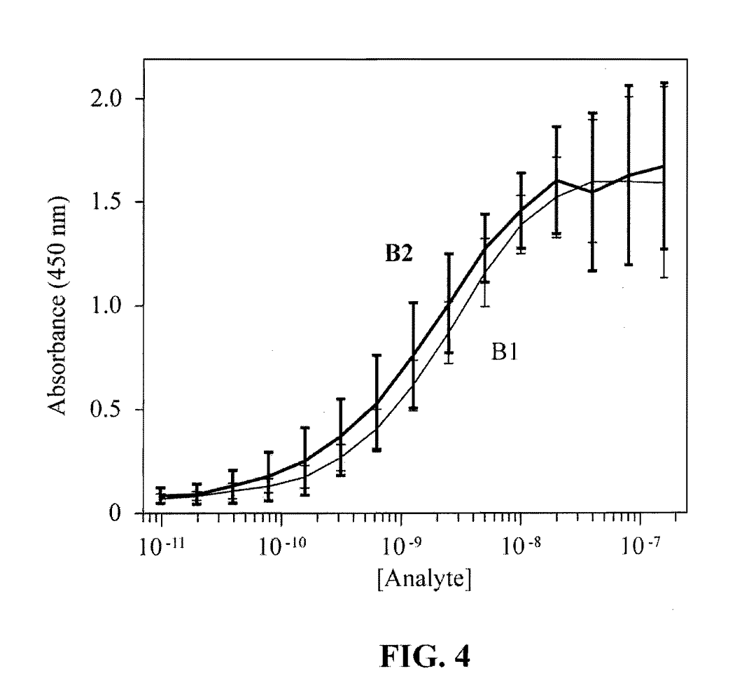

In order to make more quantifiable determinations of relative binding, an enzyme linked immunosorbent assay (ELISA) was designed to compare saturation curves for the binding of NTa3, the N-terminal domain of the osteoclast specific isoform, with B1 and B2. Saturation curves, shown in FIG. 4, again indicated that differences between NTa3/B1 binding and NTa3/B2 binding were not significantly different. Apparent K_{d} values were 2.3±0.9 nM (n=6) for NTa3/B1 and 2.4±1.2 nM (n=12) for NTa3/B2

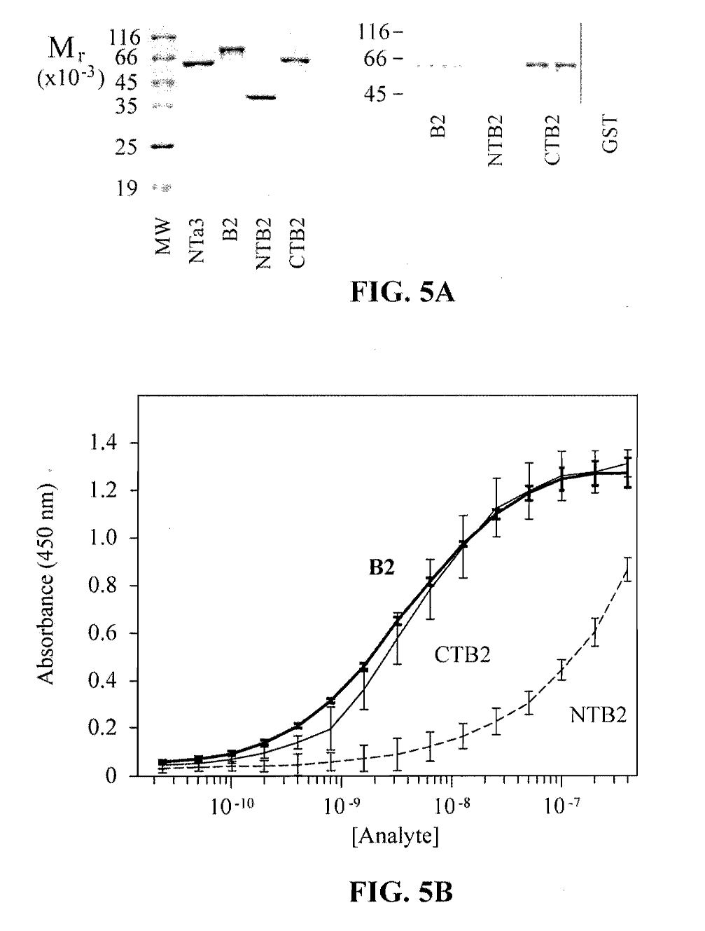

Since a3 and B2 subunit isoforms are expressed in the plasma membranes of active osteoclasts, attention was focused on that pair, and experiments were done to further delineate the sites of binding. Holliday and co-workers split B2 into an N-terminal domain (a.a. 1-117) and a C-terminal domain (a.a. 118-511) and showed that actin binding was confined to the N-terminal domain. In the present work, similar constructs were prepared (Table 2) to express GST fusions of N-terminal and C-terminal domains of B2, and ELISA analysis was performed. In contrast to actin binding, a3 binding to the B2 subunit was confined to the C-terminal domain (FIGS. 5A and 5B). Binding of NTa3 to the C-terminal domain of B2 had an apparent K_{d} of 4.1±1.7 nM (n=6), which was not significantly different from binding to the full-length subunit, whereas binding to the N-terminal domain was reduced significantly, with a K_{d} of 230±50 nM, n=6).

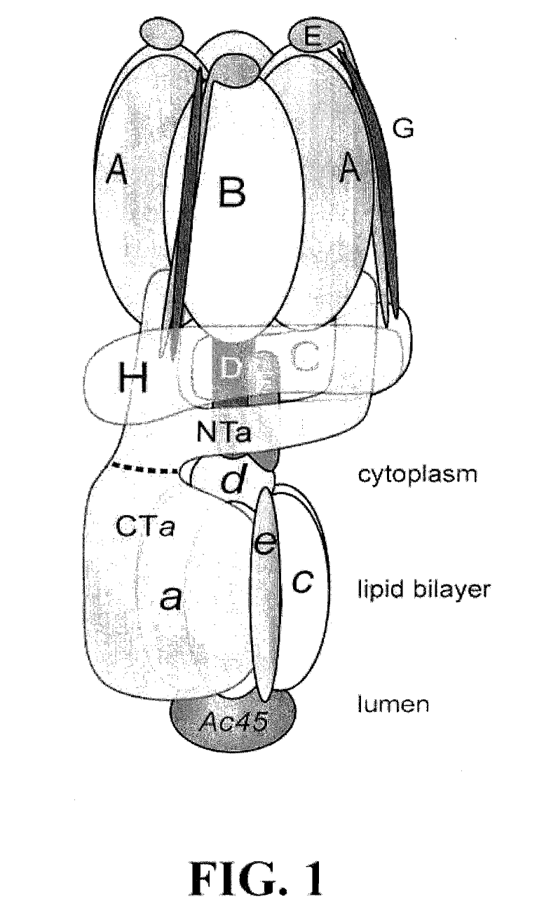

The present work identified binding sites for N-terminal domains of a subunit isoforms in the C-terminal domains of B1 and B2 subunit isoforms of mouse V-ATPase. While true affinities are difficult to determine by ELISA, apparent dissociation constants for the purpose of comparison can be calculated from half-maximal binding values derived from saturation curves. For the experiments presented here, the calculated apparent K_{d} values, 2.3 nM for NTa3/B1 and 2.4 nM for NTa3/B2 were not significantly different. Since ELISA tends to underestimate binding affinities, these values likely represent minimum affinities, suggesting that NTa3/B binding is a high affinity interaction having biological significance. In comparison, the reported affinity for F-actin binding to the B2 subunit is approximately 20-fold lower, with a K_{d} of 55 nM {Lee, 1999 #1472}. Likewise, binding of NTa3 to the C-terminal domain of B2 was not significantly different from binding to the full-length subunit, whereas binding to the N-terminal domain was reduced approx. 60-fold, with a K_{d} 0.23±0.05 μM, indicating negligible interaction with NTa3 in comparison with the contribution of the C-terminal domain. Based on this study a proposed conformational structure for V-ATPase is shown in FIG. 1.

### Example 2

### ELISA System for Screening for Modulators of the V-ATPase N-terminal Domain a3 Subunit and B Subunits or C-Terminal Domains Thereof

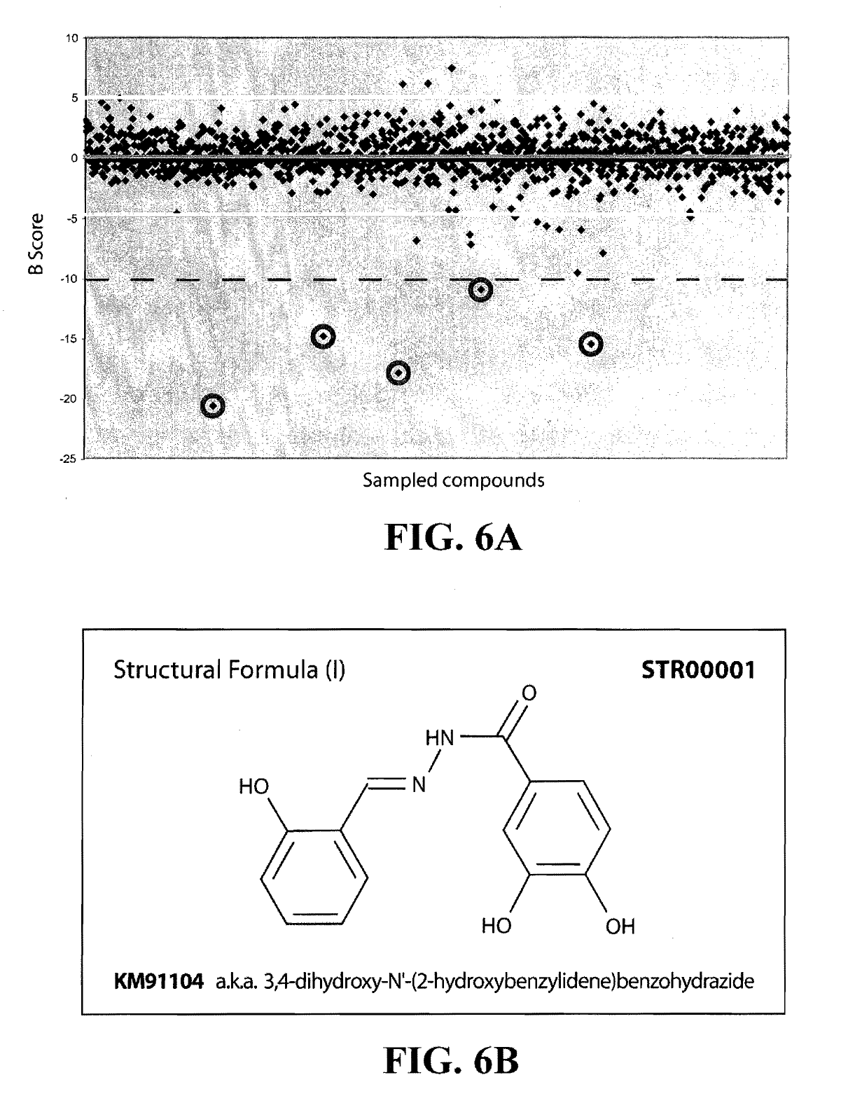

The ELISA assay described in Example 1 was used with the exception that it was modified to add test compounds. A lead compound (see Example 3) was selected from a library of 10,000 compounds (DiverSet, Chembridge Inc.). Results for screening are shown in FIG. 6A, with lead candidate (described in FIG. 6B) identification by B score

### Example 3

### In vitro Testing of 3,4-dihydroxy-N′-(2-hydroxybenzylidene)benzohydrazide

The lead compound 3,4-dihydroxy-N′-(2-hydroxybenzylidene)benzohydrazide identified in the ELISA system described in Example 2 was tested in in vitro assays. The toxicity of the lead compound on the murine RAW264.7 cells was first examined. Positive results led us to the next set of experiments. Assays were carried out to examine the effect of this compound on osteoclast formation and maturation. Resorption assays were carried out to identify the potential of the lead compound as an antiresorptive. The resorption assay revealed that the lead compound inhibited resorption without affecting osteoclast formation.

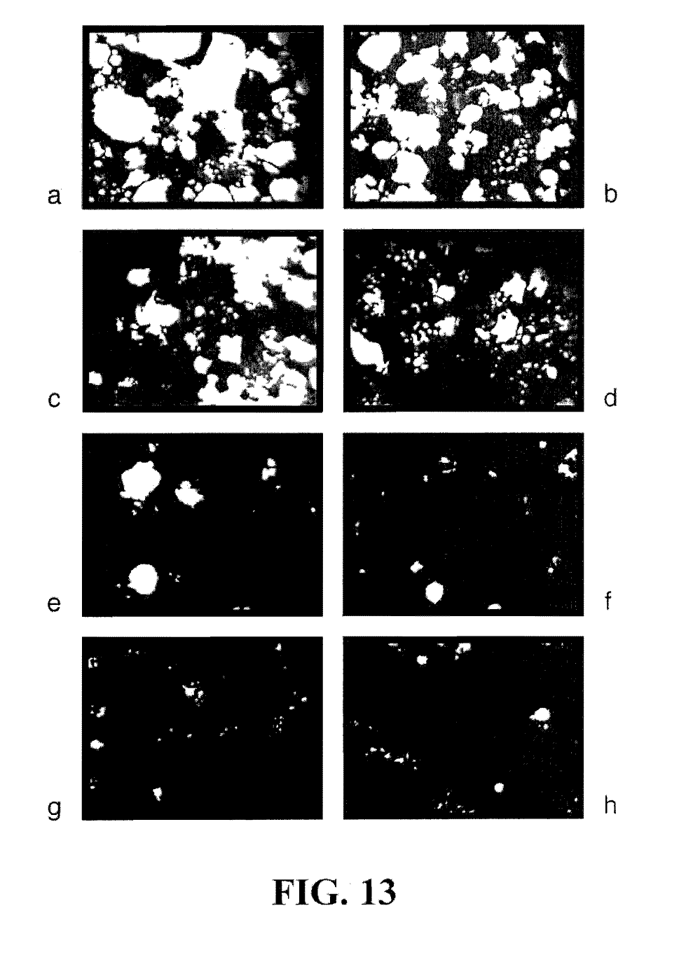

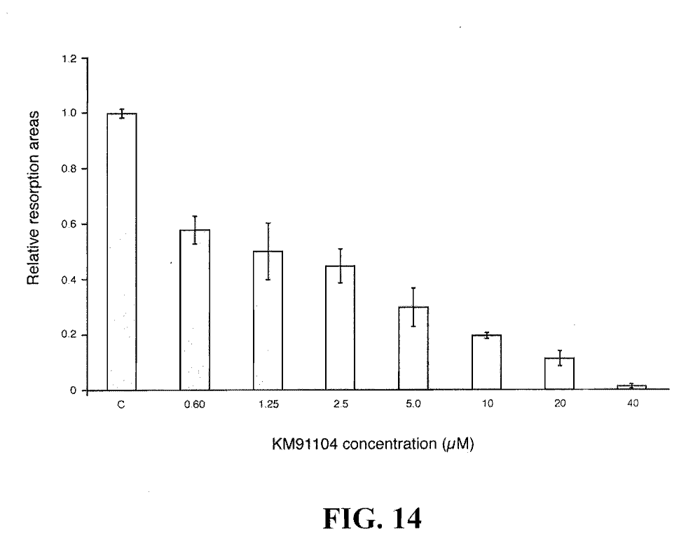

Bone resorption (FIGS. 13 and 14) was assayed using the following method. RAW 264.7 cells were differentiated on synthetic mineralized surfaces in 96 well Corning Osteo Assay Surface plates with 100 ng/ml RANK ligand. Wells were simultaneously treated with the lead compound. Cells were cultured for 5 days in 2004 of α-MEM. The complete medium was changed on the third day. On day 5, cells were removed using 50% bleach solution. Plates were dried and imaged using digital brightfield photomicrograpy. Image analysis was carried out using ImageJ.

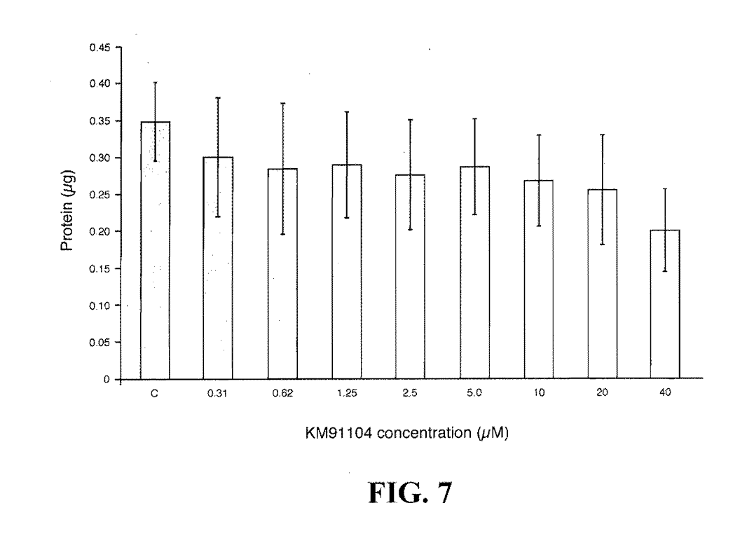

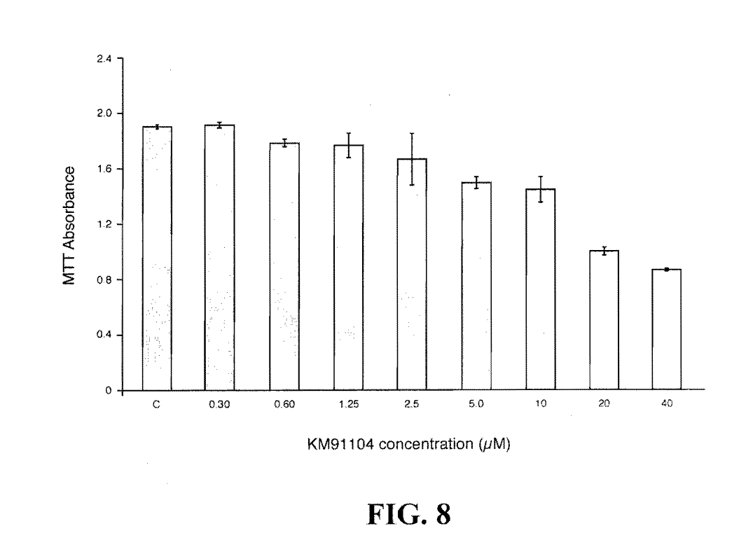

As discussed in more detail below and as illustrated in FIGS. 7 to 12 the compound was found to have low toxicity, with little effect on osteoclast formation and differentiation, yet eliciting a significantly reduced acid secretion. These properties are deemed ideal for a therapeutic for the treatment of pathological bone loss and prevention of cancer metastasis.

The following is a summary of the results:

FIG. 7 illustrates that <20 μM of the lead compound does not affect total protein.

FIGS. 13 and 14 illustrate that the lead compound prevents bone resorption in vitro.

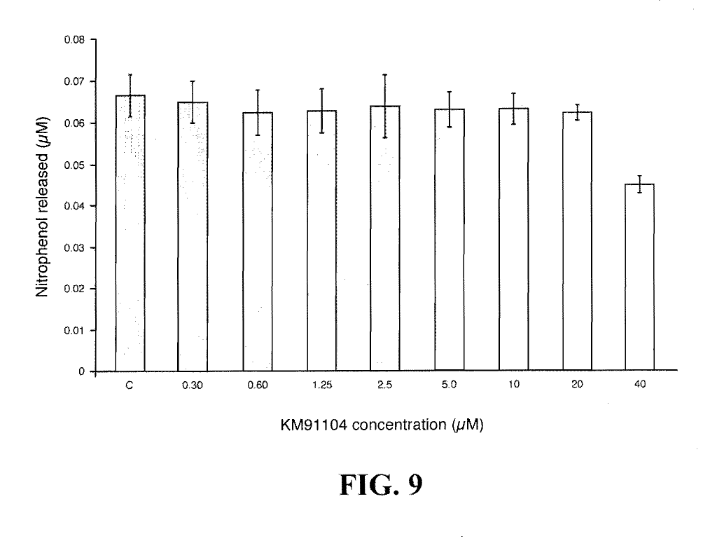

FIG. 9 illustrates that <20 μM of the lead compound does not effect mature osteoclast formation.

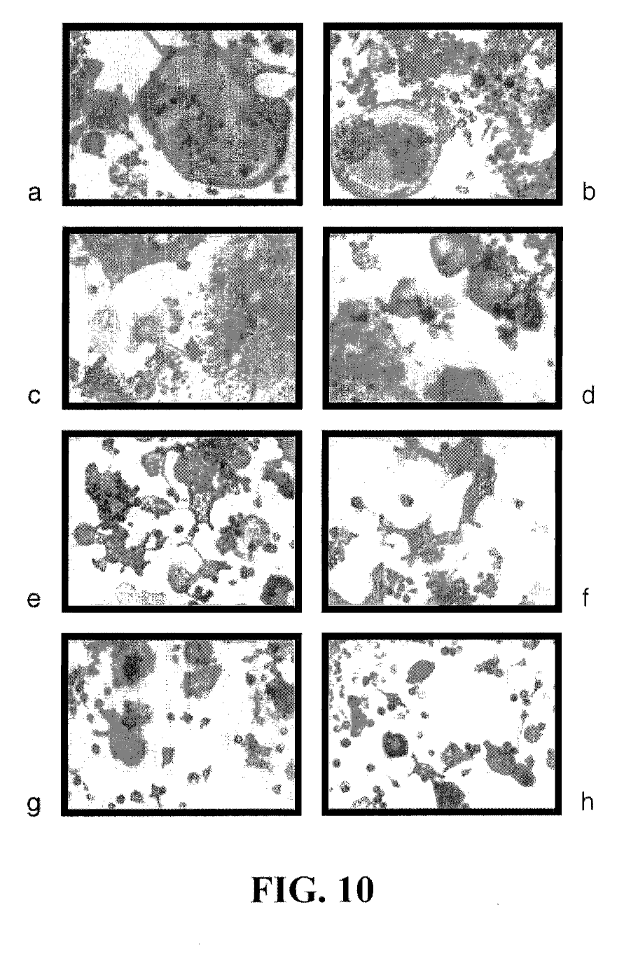

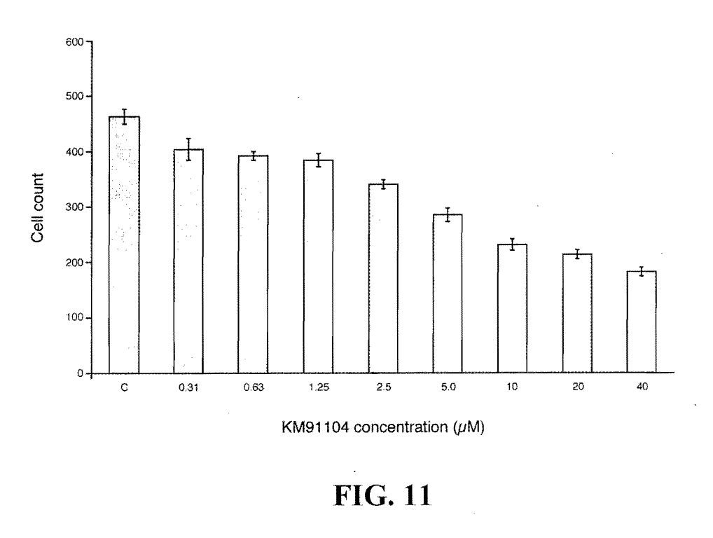

FIGS. 10 and 11 illustrate that <5 μM of the lead compound does not affect osteoclast fusion.

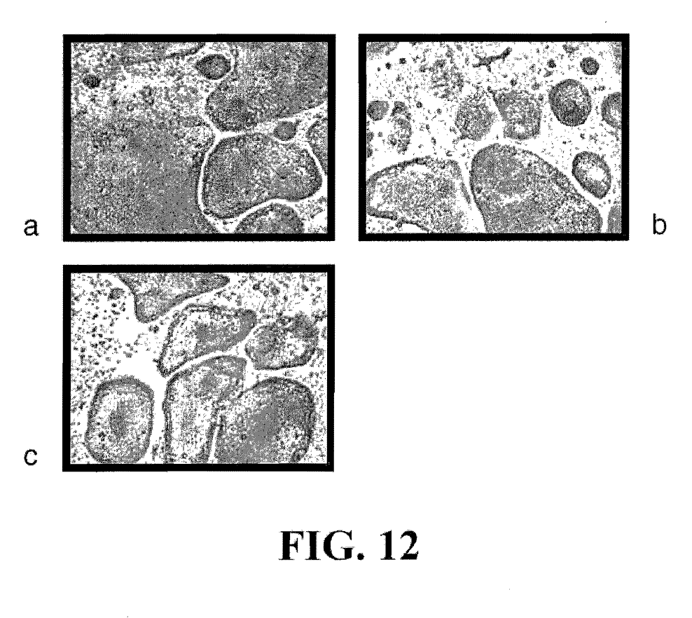

FIG. 12 illustrates that 1.25 μM of the lead compound does not affect BMM derived osteoclast maturation.

Thus the lead compound inhibits acid secretion in murine osteoclasts, preventing them from resorbing bone. It has also been shown to inhibit acid secretion from osteoclasts, but it has limited effect on their ability to differentiate or undergo fusion at a concentration that is efficacious in inhibiting acid secretion. Further the compound has no discernable effect on growth or metabolic activity at a concentration that is efficacious in inhibiting acid secretion.

The lead compound will be further validated in human osteoclasts differentiated from CD14+ monocytes sorted from peripheral blood mononuclear cells. Selectivity of inhibitors towards osteoclasts will be studied using an in situ cytochemical assay for bafilomycin-sensitive V-ATPase activity, using sections of osteoclastoma and control tissues. The compound or an analog or derivative thereof is expected to inhibit osteoclast V-ATPase at orders of magnitude lower concentrations than V-ATPases in control tissues. In vivo animal studies will be combined with toxicology studies as part of the pre-clinical development. To address prevention of bone loss in inflammatory arthritis, an adjuvant-induced rat model, or anti-collagen antibody treated mouse model will be used.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the methods etc. which are reported therein which might be used in connection with the invention. Nothing herein is to be construed as an admission that the invention is not entitled to antedate such disclosure by virtue of prior invention.

TABLE 1

| Compound No./Name | Chemical Structure |
|---|---|
| 1 3,4-dihydroxy-N′-(2- hydroxybenzylidene)benzohydrazide | 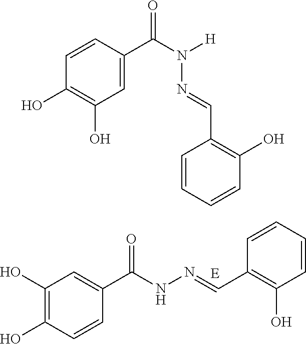 |
| 2 3,4-Dihydroxy-N′-(2- hydroxybenzylidene)benzohydrazide-methanol- water (2/1/3) | 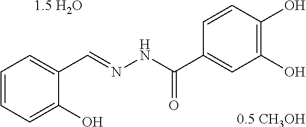 |
| 3 Benzoic acid, 3,4-dihydroxy-, 2-[(2,5- dihydroxyphenyl)methylene]hydrazide | 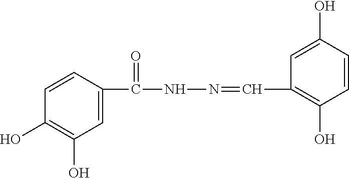 |
| 4 Benzoic acid, 3,4,5-trihydroxy-, 2-[(2- hydroxyphenyl)methylene]hydrazide | 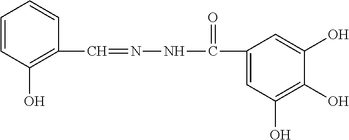 |
| 5 Benzoic acid, 4-hydroxy-3-methoxy-, 2-[(2- hydroxyphenyl)methylene]hydrazide | 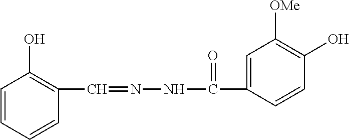 |
| 6 Benzoic acid, 3,4-dihydroxy-, 2-[(5-fluoro-2- hydroxyphenyl)methylene]hydrazide | 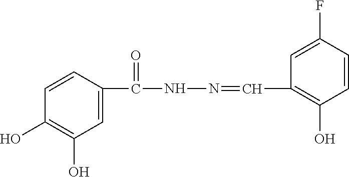 |
| 7 Benzoic acid, 3,4-dihydroxy-, 2-[1-[2-[2- hydroxy-3-[(1- methylethyl)amino]propoxy]phenyl]ethylidene] hydrazide, hydrochloride (1:1) | 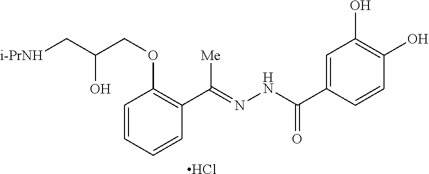 |
| 8 Benzoic acid, 4-hydroxy-3-methoxy-, 2-[(2- ethoxyphenyl)methylene]hydrazide | 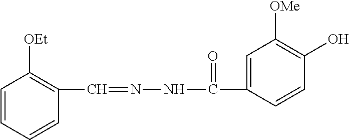 |
| 9 Benzoic acid, 3,4-dimethoxy-, 2-[(2-hydroxy-5- methylphenyl)methylene]hydrazide | 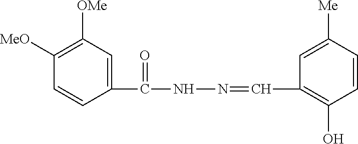 |
| 10 Benzoic acid, 4-hydroxy-3-methoxy-, 2-[(2- hydroxy-1-naphthalenyl)methylene]hydrazide, compd. with methanol | 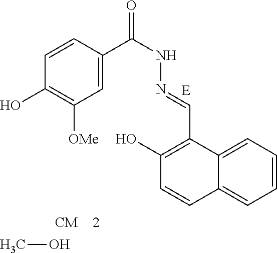 |
| 11 Benzoic acid, 3,4-dimethoxy-, 2-[(2- methoxyphenyl)methylene]hydrazide | 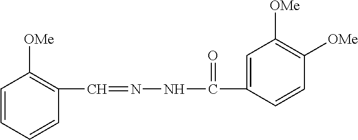 |
| 12 Benzoic acid, 3,4-dihydroxy-, 2-[(2-hydroxy-5- methylphenyl)methylene]hydrazide | 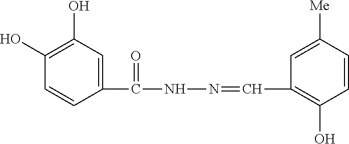 |
| 13 | 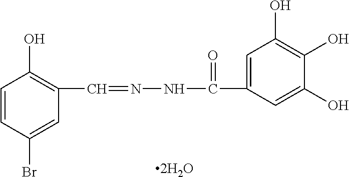 |
| 14 Benzoic acid, 3,4-dimethoxy, 2-[1-(2- hydroxyphenyl)ethylidene]hydrazide | 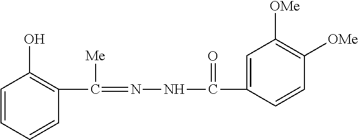 |
| 15 Benzoic acid, 4-hydroxy-3-methoxy-, 2-[(2,4- dihydroxyphenyl)methylene]hydrazide | 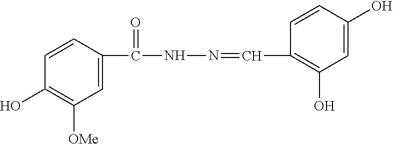 |
| 16 Benzoic acid, 3,4,5-trihydroxy-, (2E)-2-[(2,4- dimethoxyphenyl)methylene]hydrazide, compd. with ethanol (1:1) | 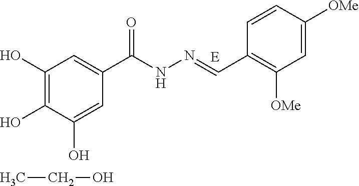 |
| 17 Benzoic acid, 3,4,5-trihydroxy-, 2-[1-(2- hydroxyphenyl)ethylidene]hydrazide | 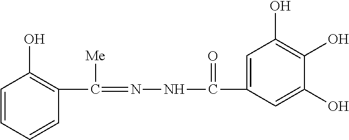 |
| 18 Benzoic acid, 3,4,5-trihydroxy-, 2-[(2,4- dihydroxyphenyl)methylene]hydrazide | 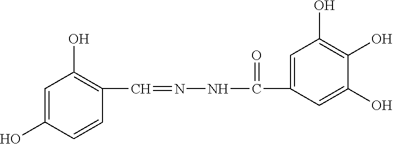 |
| 19 Benzoic acid, 3,4-dihydroxy-, (2E)-2-[(2- hydroxyphenyl)methylene]hydrazide, compd. with methanol, hydrate (2:1:3) | 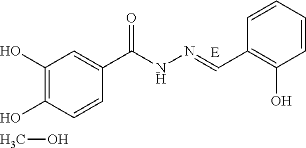 |
| 20 Benzoic acid, 3,4-dimethoxy-, 2-[(2- hydroxyphenyl)methylene]hydrazide | 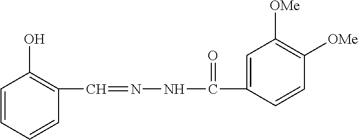 |
| 21 Benzoic acid, 3,4,5-trihydroxy-, 2-[(2- methoxyphenyl)methylene]hydrazide | 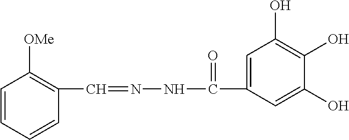 |
| 22 Benzoic acid, 3,4-dihydroxy-, (2E)-2-[(2- hydroxyphenyl)methylene]hydrazide | 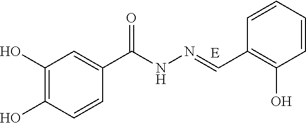 |
| 23 Benzoic acid, 3,4-dihydroxy-, 2-[1-(2- hydroxyphenyl)ethylidene]hydrazide | 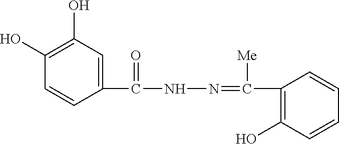 |
| 24 Benzoic acid, 4-hydroxy-3-methoxy-, 2-[(2,5- dihydroxyphenyl)methylene]hydrazide | 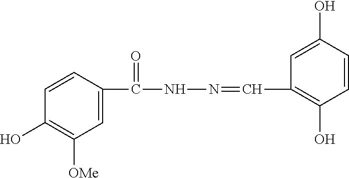 |
| 25 | 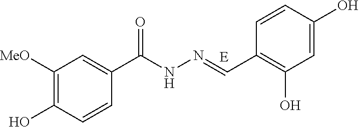 |
| 26 Benzoic acid, 3,4-dihydroxy-, 2-[(2- hydroxyphenyl)methylene]hydrazide | 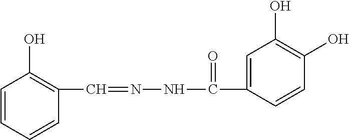 |
| 27 Benzoic acid, 3,4-diethoxy-, 2-(2- hydroxybenzoyl)hydrazide | 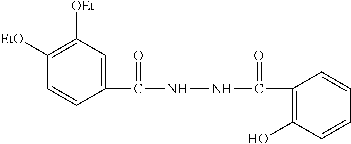 |
| 28 Benzoic acid, 3,4-dimethoxy-, 2-[[2-(2-propen-1- yloxy)phenyl]methylene]hydrazide | 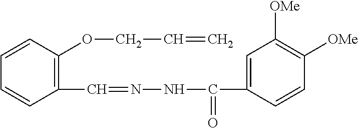 |
| 29 Benzoic acid, 3,4-dimethoxy-, 2-[(2,4- dihydroxyphenyl)methylene]hydrazide | 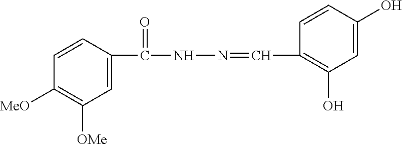 |
| 30 Benzoic acid, 2-hydroxy-3-methyl-, 2-(3,4- dimethoxybenzoyl)hydrazide | 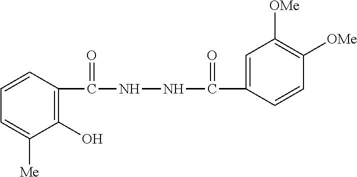 |
| 31 Benzoic acid, 3,4,5-trihydroxy-, (2E)-2-[(2,4- dimethoxyphenyl)methylene]hydrazide | 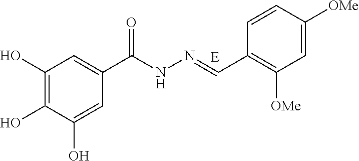 |
| 32 Benzoic acid, 3,4-dihydroxy-, 2-[(2-methoxy-1- naphthalenyl)methylene]hydrazide | 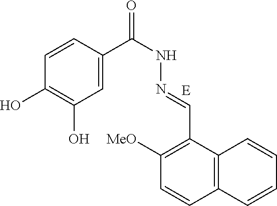 |
| 33 Benzoic acid, 3,4,5-trihydroxy-, 2-[(2- methoxyphenyl)methylene]hydrazide | 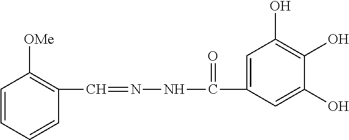 |
| 34 Benzoic acid, 3,4-dimethoxy-, 2-[(5-chloro-2- hydroxyphenyl)methylene]hydrazide | 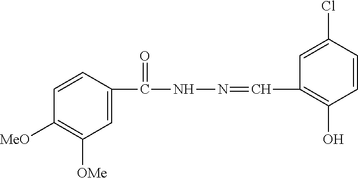 |
| 35 Benzoic acid, 3,4-dihydroxy-, 2-[(2-methoxy-1- naphthalenyl)methylene]hydrazide | 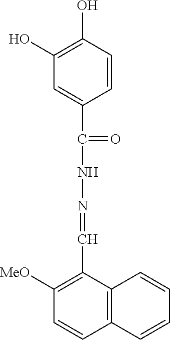 |
| 36 Benzoic acid, 4-hydroxy-3-methoxy-, (2E)-2- [(2,4-dimethoxyphenyl)methylene]hydrazide | 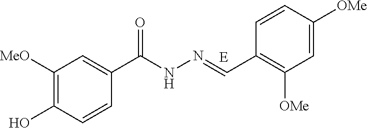 |
| 37 Benzoic acid, 4-hydroxy-3,5-dimethoxy-, 2-[(2- methoxyphenyl)methylene]hydrazide | 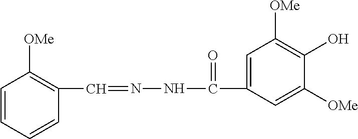 |
| 38 Benzoic acid, 3,4-dimethoxy-, 2-[(4-ethoxy-2- hydroxyphenyl)methylene]hydrazide | 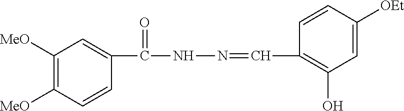 |
| 39 Benzoic acid, 3,4-dimethoxy-, 2-[(2- ethoxyphenyl)methylene]hydrazide | 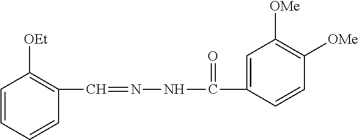 |
| 40 Benzoic acid, 3,4,5-trimethoxy-, 2-[(2- hydroxyphenyl)methylene]hydrazide | 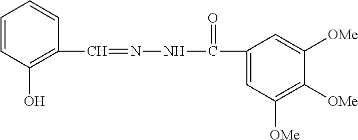 |
| 41 Benzoic acid, 4-ethoxy-3-methoxy-, 2-(2- methoxybenzoyl)hydrazide | 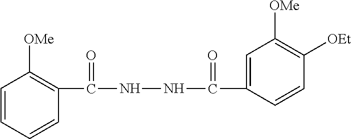 |
| 42 Benzoic acid, 4-ethoxy-3-methoxy-, 2-(2- methoxybenzoyl)hydrazide | 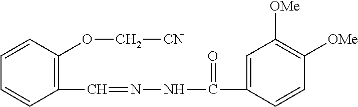 |
| 43 Benzoic acid, 4-hydroxy-3-methoxy-, 2-[1-(2- hydroxyphenyl)ethylidene]hydrazide | 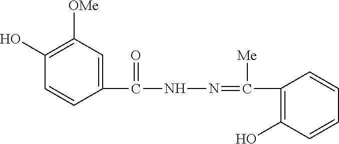 |
| 44 Benzoic acid, 3,4-dimethoxy-, 2-(2- hydroxybenzoyl)hydrazide | 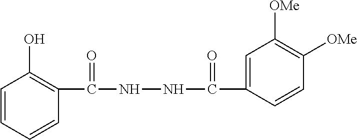 |
| 45 Benzoic acid, 3,4-dihydroxy-, (2E)-2-[(2- hydroxyphenyl)methylene]hydrazide, compd. with methanol, hydrate (2:1:3) | 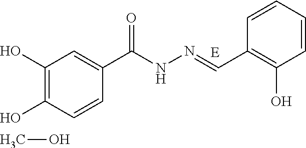 |
| 46 Benzoic acid, 3-methoxy-4-(1-methylethoxy)-, 2- [(2,4-dihydroxyphenyl)methylene]hydrazide | 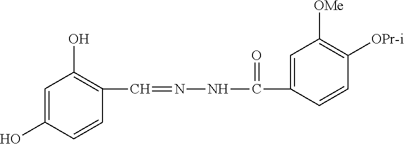 |
| 47 Benzoic acid, 3,4-dimethoxy-, 2-[1-(2- hydroxyphenyl)propylidene]hydrazide | 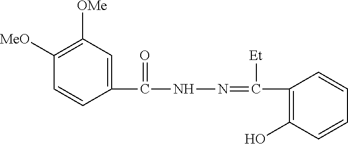 |
| 48 Benzoic acid, 4-hydroxy-3-methoxy-, 2-[1-(2,4- dihydroxyphenyl)ethylidene]hydrazide | 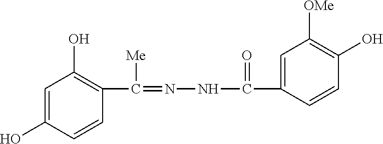 |
| 49 Benzoic acid, 3,4-dihydroxy-, 2-[1-(2- hydroxyphenyl)ethylidene]hydrazide | 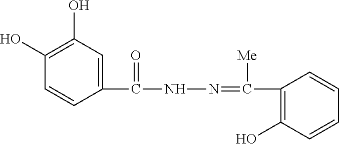 |
| 50 Benzoic acid, 4-hydroxy-3-methoxy-, 2-[(3- hydroxy-2-methoxyphenyl)methylene]hydrazide | 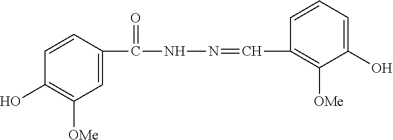 |
| 51 Benzoic acid, 3,4-dimethoxy-, 2-[(2,4,6- trihydroxyphenyl)methylene]hydrazide | 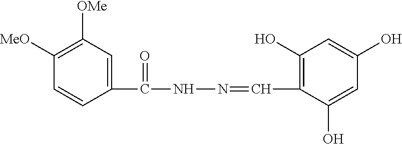 |
| 52 Benzoic acid, 3,4-dimethoxy-, 2-[1-(2- methoxyphenyl)ethylidene]hydrazide | 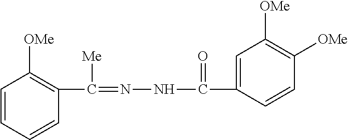 |
| 53 Benzoic acid, 3,4-dimethoxy-, 2-[(2,4- dimethoxyphenyl)methylene]hydrazide | 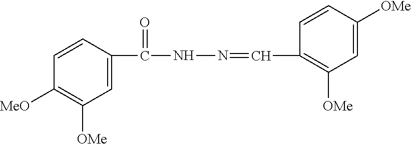 |
| 54 Benzoic acid, 3,4,5-trihydroxy-, 2-[(2- hydroxyphenyl)methylene]hydrazide | 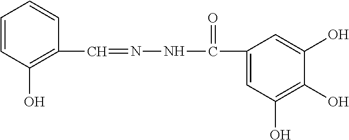 |
| 55 Benzoic acid, 3,4,5-trimethoxy-, (2E)-2-[(2- hydroxyphenyl)methylene]hydrazide | 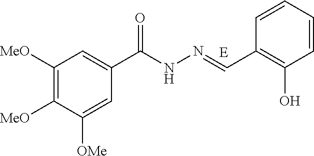 |
| 56 Benzoic acid, 3,4-dimethoxy-, 2-[(2-hydroxy-4- methoxyphenyl)methylene]hydrazide | 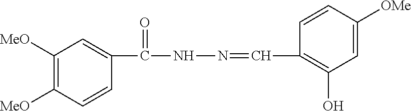 |
| 57 Benzoic acid, 4-hydroxy-3-methoxy-, 2-[(2- hydroxyphenyl)methylene]hydrazide | 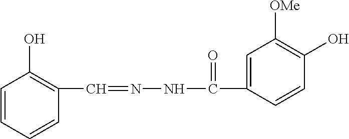 |
| 58 Benzoic acid, 3,4-dimethoxy-, 2-(2- methoxybenzoyl)hydrazide | 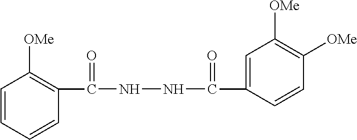 |
| 59 Benzoic acid, 3,4-dihydroxy-, (2E)-2-[(2- hydroxyphenyl)methylene]hydrazide | 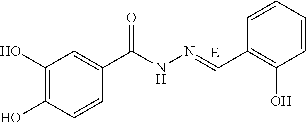 |
| 60 Benzoic acid, 3-methoxy-4-(1-methylethoxy)-, 2- [(2-hydroxyphenyl)methylene]hydrazide | 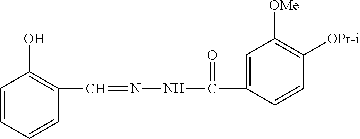 |
| 61 Benzoic acid, 3,4,5-trihydroxy-, 2-[(2- ethoxyphenyl)methylene]hydrazide | 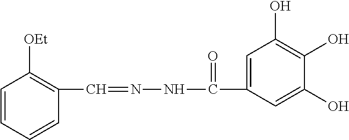 |
| 62 Benzoic acid, 4-hydroxy-3-methoxy-, 2-[(2,4- dimethoxyphenyl)methylene]hydrazide | 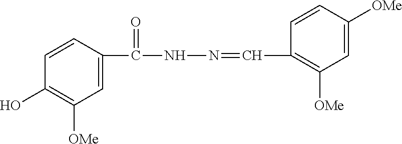 |
| 63 Benzoic acid, 3,4-dihydroxy-, 2-[(2- hydroxyphenyl)methylene]hydrazide | 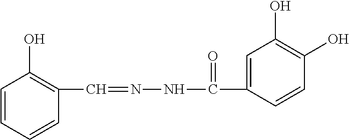 |
| 64 | 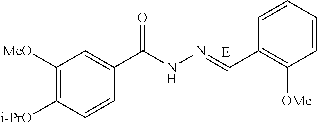 |
| 65 Benzoic acid, 3,4,5-trihydroxy-, 2-[(2-hydroxy-3- nitrophenyl)methylene]hydrazide | 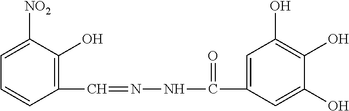 |
| 66 Benzoic acid, 3,4,5-trimethoxy-, 2-[(2,3- dihydroxyphenyl)methylene]hydrazide | 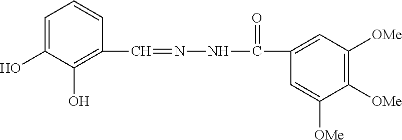 |
| 67 Benzoic acid, 3,4-dimethoxy-, 2-[(2,3- dimethoxyphenyl)methylene]hydrazide | 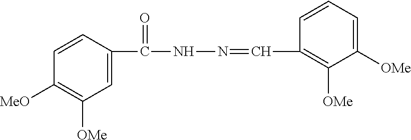 |
| 68 Benzoic acid, 3,4-dihydroxy-, 2-[1-[2-[2- hydroxy-3-[(1- methylethyl)amino]propoxy]phenyl]ethylidene] hydrazide, hydrochloride (1:1) | 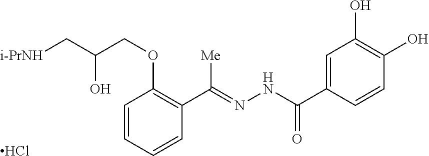 |
| 69 Benzoic acid, 3,4-dimethoxy-, 2-[(2,5- dihydroxyphenyl)methylene]hydrazide | 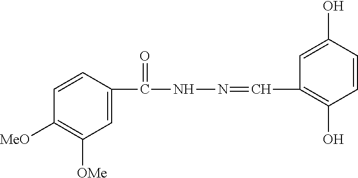 |
| 70 Benzoic acid, 3,4,5-trihydroxy-, 2-[(2,4- dihydroxyphenyl)methylene]hydrazide | 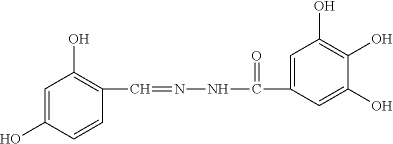 |
| 71 Benzoic acid, 4-hydroxy-3-methoxy-, 2-[(5- chloro-2-hydroxyphenyl)methylene]hydrazide | 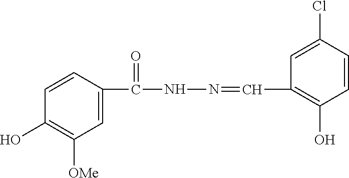 |
| 72 Benzoic acid, 3,4,5-trihydroxy-, 2-[1-(2- hydroxyphenyl)ethylidene]hydrazide | 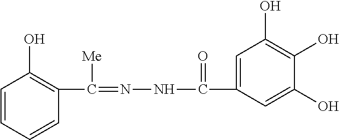 |
| 73 Benzoic acid, 3-methoxy-4-propoxy-, 2-(2- hydroxybenzoyl)hydrazide | 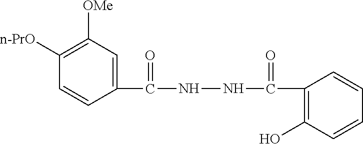 |
| 74 Benzoic acid, 3,4-dimethoxy-, 2-[1-(5-fluoro-2- hydroxyphenyl)ethylidene]hydrazide | 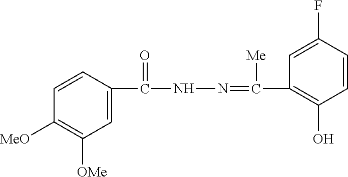 |
| 75 Benzoic acid, 3,4,5-trihydroxy-, 2-[(1-hydroxy-2- naphthalenyl)methylene]hydrazide | 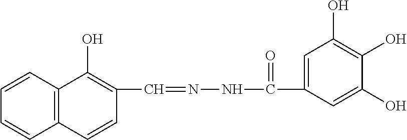 |
| 76 Benzoic acid, 4-hydroxy-3-methoxy-, 2-[(2,3- dimethoxyphenyl)methylene]hydrazide | 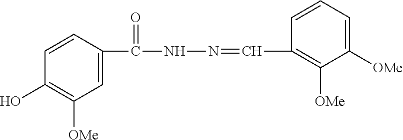 |
| 77 Benzoic acid, 3,4-dihydroxy-, 2-[(2-hydroxy-5- nitrophenyl)methylene]hydrazide | 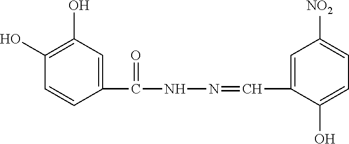 |
| 78 Benzoic acid, 3,4-dimethoxy-, 2-[1-(2,5- dihydroxyphenyl)ethylidene]hydrazide | 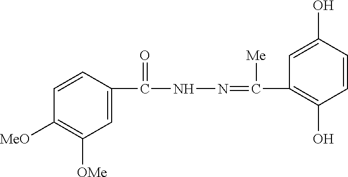 |
| 79 Benzoic acid, 3,4,5-trimethoxy-, 2-(2- hydroxybenzoyl)hydrazide | 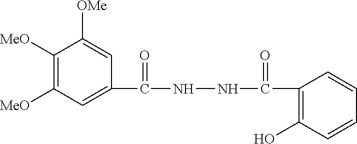 |
| 80 Benzoic acid, 3,4-dimethoxy-, 2-[1-(2-hydroxy- 4-methoxyphenyl)ethylidene]hydrazide | 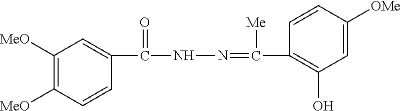 |
| 81 Benzoic acid, 3,4-dimethoxy-, 2-[(2- methoxyphenyl)methylene]hydrazide | 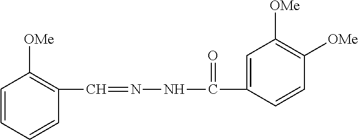 |
| 82 Benzoic acid, 4-hydroxy-3-methoxy-, 2-[(2- hydroxy-1-naphthalenyl)methylene]hydrazide, compd. with methanol (1:1) | 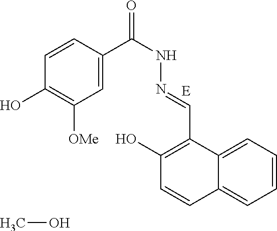 |
| 83 Benzoic acid, 3,4-dihydroxy-, 2-[(2,5- dihydroxyphenyl)methylene]hydrazide | 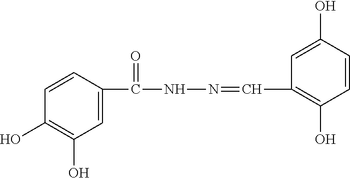 |
| 84 Benzoic acid, 3,4,5-trihydroxy-, 2-[1-(2- hydroxyphenyl)propylidene]hydrazide | 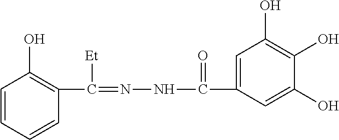 |
| 85 Benzoic acid, 4-hydroxy-3-methoxy-, 2-[(2,5- dimethoxyphenyl)methylene]hydrazide | 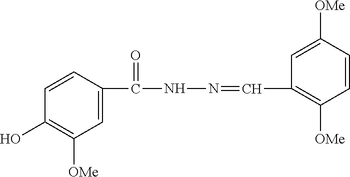 |
| 86 Benzoic acid, 3,4,5-trihydroxy-, 2-[(2-hydroxy-3- methoxyphenyl)methylene]hydrazide | 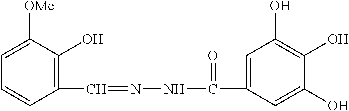 |
| 87 Benzoic acid, 3,4-dimethoxy-, 2-[1-(2-hydroxy- 5-methoxyphenyl)ethylidene]hydrazide | 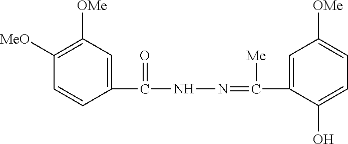 |
| 88 Benzoic acid, 3,4,5-trihydroxy-, 2-(2- methoxybenzoyl)hydrazide | 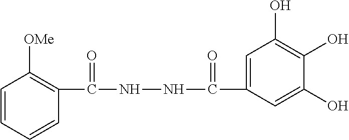 |
| 89 Benzoic acid, 3,4,5-trihydroxy-, 2-[(2,5- dimethoxyphenyl)methylene]hydrazide | 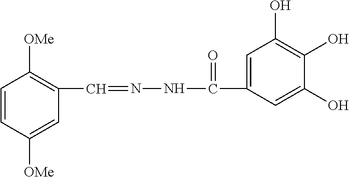 |
| 90 Benzoic acid, 4-hydroxy-3-methoxy-, 2-[(2- hydroxy-3-methoxyphenyl)methylene]hydrazide | 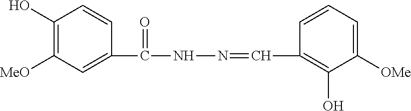 |
| 91 Benzoic acid, 3,4,5-trimethoxy-, 2-[(2- methoxyphenyl)methylene]hydrazide | 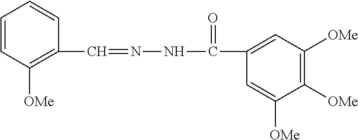 |
| 92 Benzoic acid, 3,4-dimethoxy-, (2Z)-2-[[2-(2- propyn-1-yloxy)phenyl]methylene]hydrazide | 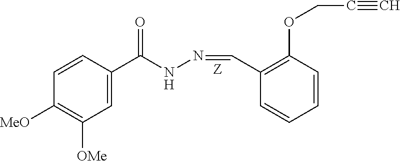 |
| 93 Benzoic acid, 3,4-dimethoxy-, 2-(2- ethoxybenzoyl)hydrazide | 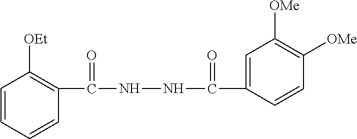 |
| 94 Benzoic acid, 3,4-dimethoxy-, 2-[(2-hydroxy-5- methoxyphenyl)methylene]hydrazide | 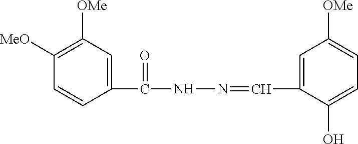 |
| 95 Benzoic acid, 3,4,5-trimethoxy-, 2-[1-(2- hydroxyphenyl)ethylidene]hydrazide | 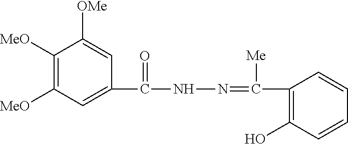 |
| 96 Benzoic acid, 4-hydroxy-3-methoxy-, (2E)-2-[(2- hydroxy-1-naphthalenyl)methylene]hydrazide | 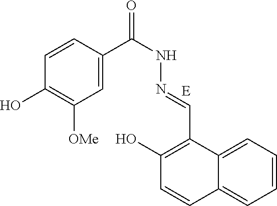 |
| 97 Benzoic acid, 3,4-dihydroxy-, 2-[(5-fluoro-2- hydroxyphenyl)methylene]hydrazide | 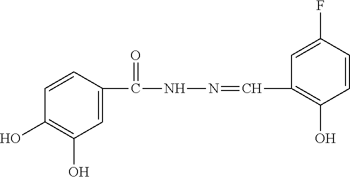 |
| 98 Benzoic acid, 3,4,5-trihydroxy-, 2-[1-(2,4- dihydroxyphenyl)ethylidene]hydrazide | 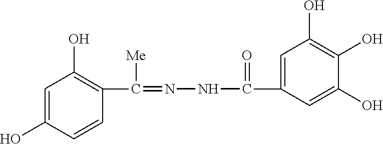 |
| 99 Benzoic acid, 4-hydroxy-3-methoxy-, 2-[(2- hydroxy-5-nitrophenyl)methylene]hydrazide | 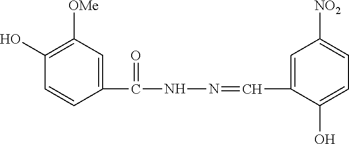 |
| 100 Benzoic acid, 3,4-dimethoxy-, 2- [(2-hydroxy-3- methoxyphenyl)methylene]hydrazide | 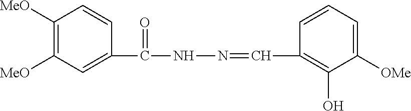 |
| 101 Benzoic acid, 4-hydroxy-3-methoxy-, 2-[(4- hydroxy-2-methoxyphenyl)methylene]hydrazide | 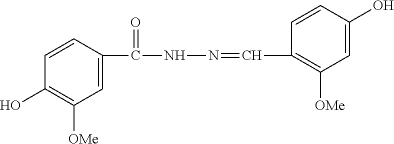 |
| 102 Benzoic acid, 3,4,5-trihydroxy-, 2-(2- ethoxybenzoyl)hydrazide | 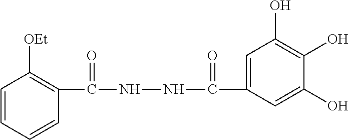 |
| 103 Benzoic acid, 3,4,5-trihydroxy-, 2-[(2,3- dimethoxyphenyl)methylene]hydrazide | 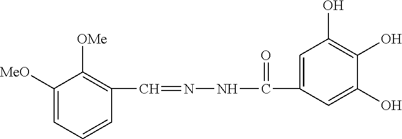 |
| 104 Benzoic acid, 3,4,5-trihydroxy-, 2-[(2-hydroxy- 1-naphthalenyl)methylene]hydrazide | 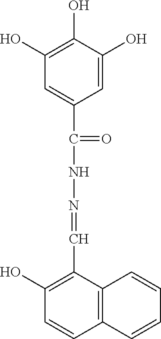 |
| 105 Benzoic acid, 3,4,5-trimethoxy-, 2-[(2,4- dihydroxyphenyl)methylene]hydrazide | 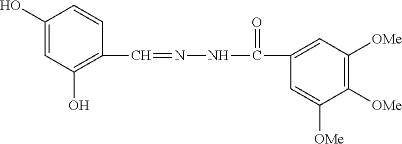 |
| 106 Benzoic acid, 3,4-dihydroxy-, 2-[(5-carboxy-2- hydroxyphenyl)methylene]hydrazide | 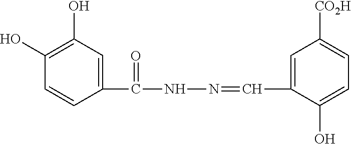 |
| 107 Benzoic acid, 4-hydroxy-3-methoxy-, 2-[(2,4- dihydroxyphenyl)methylene]hydrazide | 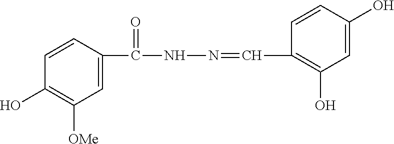 |
| 108 Benzoic acid, 3,4-dimethoxy-, 2-[1-(2,4- dihydroxyphenyl)ethylidene]hydrazide | 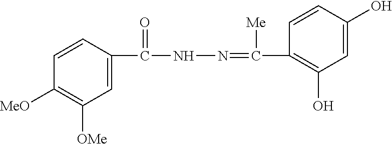 |
| 109 Benzoic acid, 3,4-dimethoxy-, 2-[1-(2- hydroxyphenyl)ethylidene]hydrazide | 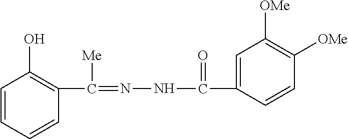 |
| 110 | 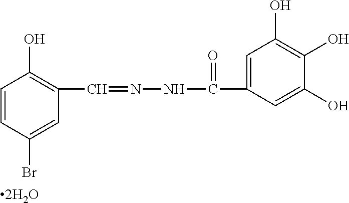 |
| 111 Benzoic acid, 3,4-dimethoxy, 2-[(2-hydroxy-5- methylphenyl)methylene]hydrazide | 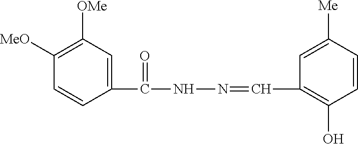 |
| 112 Benzoic acid, 3,4-dimethoxy, 2-[[2-(1- methylethoxy)phenyl]methylene]hydrazide | 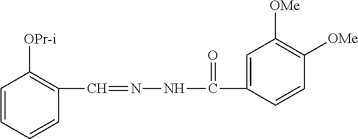 |
| 113 Benzoic acid, 4-hydroxy-3-methoxy-, 2-[(2- ethoxyphenyl)methylene]hydrazide | 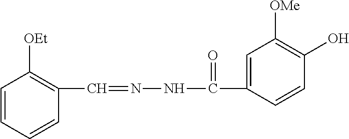 |
| 114 Benzoic acid, 3,4-dimethoxy-, 2-[[2-hydroxy-3- (2-propen-1-yl)phenyl]methylene]hydrazide | 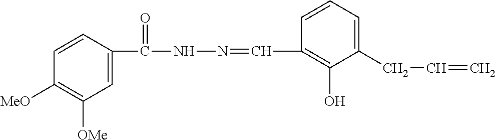 |
| 115 | 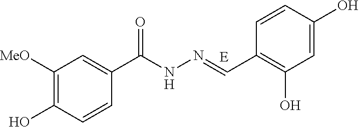 |
| 116 Benzoic acid, 4-hydroxy-3,5-dimethoxy-, 2-[1- (2-hydroxyphenyl)ethylidene]hydrazide | 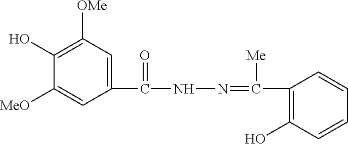 |
| 117 Benzoic acid, 3,4-dimethoxy-, 2-[(2,3,4- trihydroxyphenyl)methylene]hydrazide | 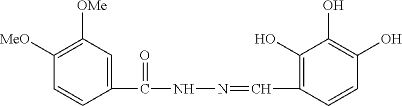 |
| 118 Benzoic acid, 3,4,5-trihydroxy-, 2-[(2,4- dimethoxyphenyl)methylene]hydrazide | 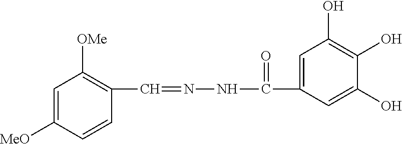 |
| 119 Benzoic acid, 3,4-dihydroxy-, 2-[(4-hydroxy-1,3- benzodioxol-5-yl)methylene]hydrazide | 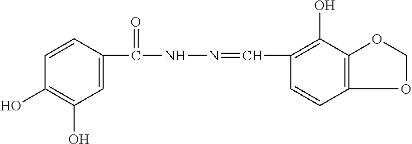 |
| 120 Benzoic acid, 3-methoxy-4-(1-methylethoxy)-, 2- (2-hydroxybenzoyl)hydrazide | 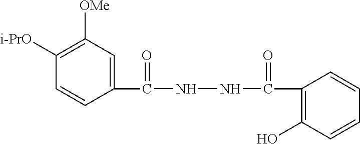 |
| 121 Benzoic acid, 4-hydroxy-3-methoxy-, 2-[(2- hydroxy-4-methoxyphenyl)methylene]hydrazide | 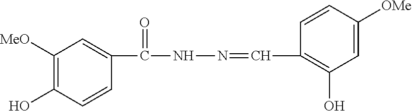 |
| 122 Benzoic acid, 4-hydroxy-3-methoxy-, 2-[(2,5- dihydroxyphenyl)methylene]hydrazide | 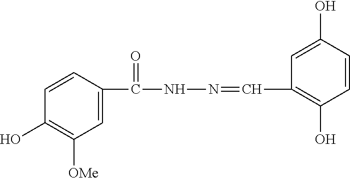 |
| 123 Benzoic acid, 3,4-dimethoxy-, 2-[(2,5- dimethoxyphenyl)methylene]hydrazide | 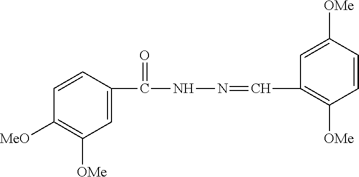 |
| 124 Benzoic acid, 3,4,5-trihydroxy-, (2E)-2-[(2,4- dimethoxyphenyl)methylene]hydrazide, compd. with ethanol (1:1) | 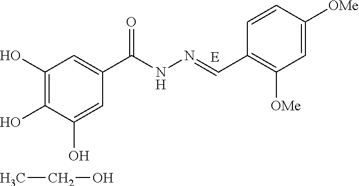 |
| 125 Benzoic acid, 3,4-dihydroxy-, 2-[(2-hydroxy-5- methylphenyl)methylene]hydrazide | 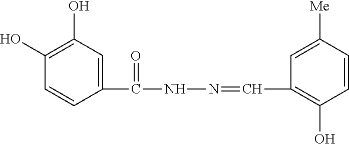 |
| 126 Benzoic acid, 3,4,5-trihydroxy-, 2-[(2-hydroxy-5- nitrophenyl)methylene]hydrazide | 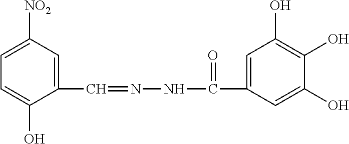 |
| 127 Benzoic acid, 3,4-dimethoxy-, 2-[(2-hydroxy-5- nitrophenyl)methylene]hydrazide | 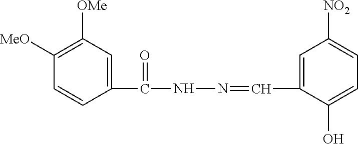 |
| 128 Benzoic acid, 3,4-dimethoxy-, 2-[(2- hydroxyphenyl)methylene]hydrazide | 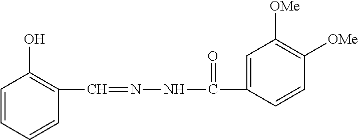 |
| 129 Benzoic acid, 4-hydroxy-3-methoxy-, (2E)-2- [2,3-dimethoxyphenyl)methylene]hydrazide | 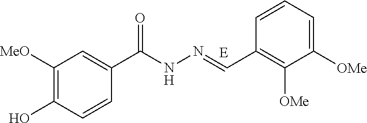 |
| 130 Benzoic acid, 4-hydroxy-3,5-dimethoxy-, 2-[(2- ethoxyphenyl)methylene]hydrazide | 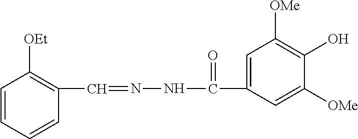 |
| 131 Benzoic acid, 3,4-dimethoxy-, 2-[(3-ethoxy-2- hydroxyphenyl)methylene]hydrazide | 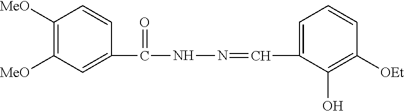 |
| 132 Benzoic acid, 3,4-dimethoxy-, 2-[(2,3- dihydroxyphenyl)methylene]hydrazide | 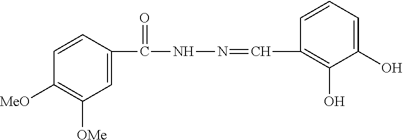 |
| 133 Benzoic acid, 3,4,5-trihydroxy-, 2-[(2,3,4- trihydroxyphenyl)methylene]hydrazide | 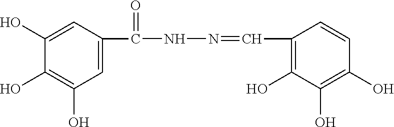 |
| 134 Benzoic acid, 3,4,5-trimethoxy-, 2-[(2,3,4- trihydroxyphenyl)methylene]hydrazide | 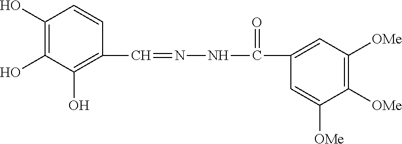 |
| 135 Benzoic acid, 3,4,5-trimethoxy-, 2-[(5-bromo-2- methoxyphenyl)methylene]hydrazide | 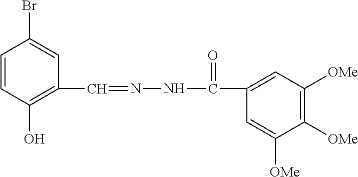 |
| 136 Benzoic acid, 3,4,5-trimethoxy-, 2-[5- (dimethylamino)-2-hydroxybenzoyl]hydrazide | 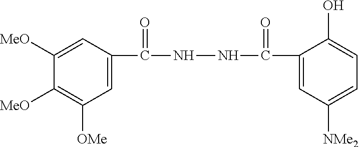 |
| 137 Benzoic acid, 3,4,5-trimethoxy-, 2-[(2,4,5- trihydroxyphenyl)methylene]hydrazide | 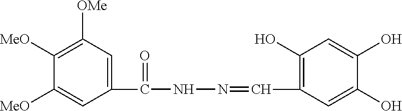 |
| 138 Benzoic acid, 3,4,5-trimethoxy-, 2-[(5-bromo-2- propoxyphenyl)methylene]hydrazide | 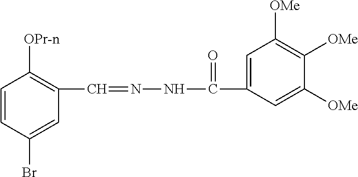 |
| 139 Benzoic acid, 3,4-dihydroxy-, 2-[(2- hydroxyphenyl)methylene]hydrazide | 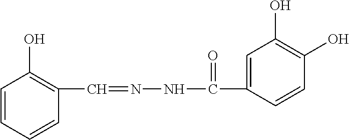 |
| 140 Benzoic acid, 3,4,5-trihydroxy-, 2-[(5-chloro-2- hydroxy-3-methoxyphenyl)methylene]hydrazide | 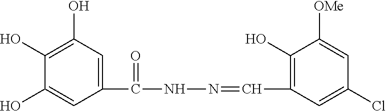 |
| 141 Benzoic acid, 3,4-dihydroxy-, 2-[(2-hydroxy-3,5- diiodophenyl)methylene]hydrazide | 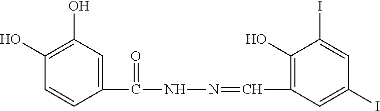 |
| 142 Benzoic acid, 3,4,5-trimethoxy-, 2-[(2,3- dimethoxyphenyl)methylene]hydrazide | 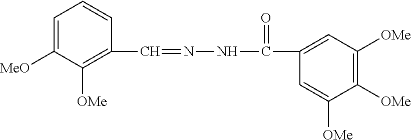 |
| 143 Benzoic acid, 3,4-dimethoxy-, 2-[[4- (diethylamino)-2- hydroxyphenyl]methylene]hydrazide | 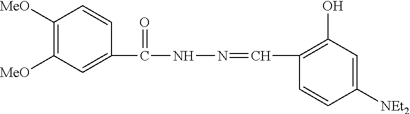 |
| 144 Benzoic acid, 3,4,5-trimethoxy-, 2-[(5-chloro-2- hydroxyphenyl)methylene]hydrazide | 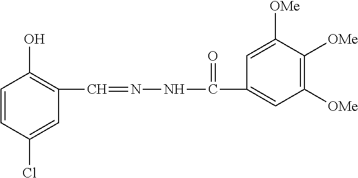 |
| 145 Benzoic acid, 3,4-dihydroxy-, 2-[(2-hydroxy-5- methylphenyl)methylene]hydrazide | 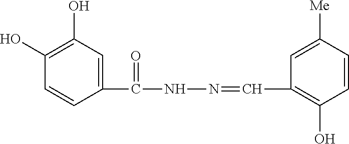 |
| 146 Benzoic acid, 4-hydroxy-3-methoxy-, 2-[(5- chloro-2-hydroxyphenyl)methylene]hydrazide | 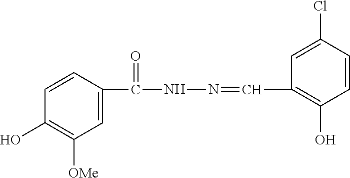 |
| 147 Benzoic acid, 3,4,5-trihydroxy-, 2-[(2,4,5- trimethoxyphenyl)methylene]hydrazide | 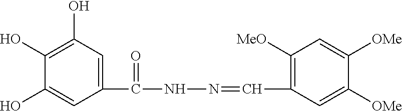 |
| 148 Benzoic acid, 3-methoxy-4-(2-propen-1- yloxy)-, 2-[(2-methoxy-1- naphthalenyl)methylene]hydrazide | 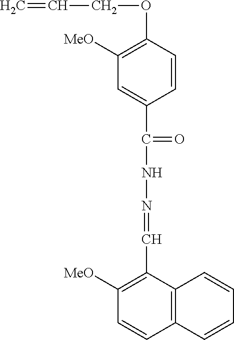 |
| 149 Benzoic acid, 3,4-dihydroxy-, 2-[(5-fluoro-2- hydroxyphenyl)methylene]hydrazide | 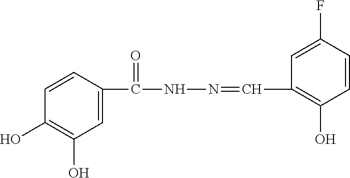 |
| 150 Benzoic acid, 4-hydroxy-3-methoxy-, 2-[(2,5- dihydroxyphenyl)methylene]hydrazide | 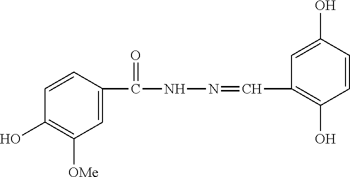 |
| 151 Benzoic acid, 3,4,5-trimethoxy-, 2-[(2,5- dimethoxyphenyl)methylene]hydrazide | 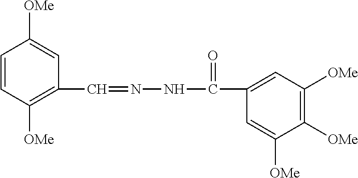 |
| 152 Benzoic acid, 3,4-dihydroxy-, 2-[(2-hydroxy-5- nitrophenyl)methylene]hydrazide | 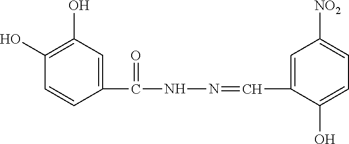 |
| 153 Benzoic acid, 3,4-dimethoxy-, 2-[(2,5- dihydroxyphenyl)methylene]hydrazide | 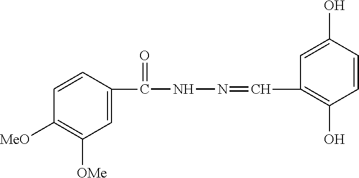 |
| 154 Benzoic acid, 3,4,5-trihydroxy-, 2-[1-(2,4- dihydroxyphenyl)ethylidene]hydrazide | 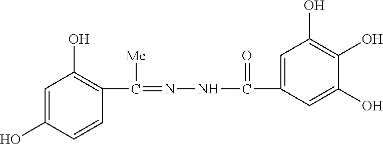 |
| 155 Benzoic acid, 3,4,5-trimethoxy-, 2-[(2,4- dimethoxyphenyl)methylene]hydrazide | 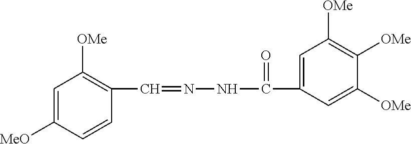 |

TABLE 2

| Subunit (domain)^{a} | | |
|---|---|---|
| [Template, PCR primers (sense/antisense)] | Expression plasmid^{b} | Product (a.a)^{c} |
| a1 (NTa1 domain) | pET32a-NTa1 | TRX-G₂-NTa1⁽¹⁻³⁹⁷⁾ |
| [RAW 264.7 cDNA, | | |
| 5′-gaattcggtggtatgggggagcttttccggagtg-3′ | | |
| (SEQ ID NO: 5) | | |
| 5′-gtcgactagatgacagtgtacggagctgggttaatctctc-3′] | | |
| (SEQ ID NO: 6) | | |
| a2 (NTa2 domain) | pET32a-NTa2 | TRX-G₂-NTa2⁽¹⁻⁴⁰²⁾ |
| [mouse brain cDNA, | | |
| 5′-gaattcggtggtatgggctctctcttccgcagc-3′ | | |
| (SEQ ID NO: 7) | | |
| 5′-gtcgactagatgatggtaaagagagctgggttcacttctctgtag-3′] | | |
| (SEQ ID NO: 8) | | |
| a3 (NTa3 domain) | pET32a-NTa3 | TRX-G₂-NTa3⁽¹⁻³⁹³⁾ |
| [pcDNA 3.1-a3, | | |
| 5′-gaattcggtggtatgggctctatgttccggagtgaagag-3′ | | |
| (SEQ ID NO: 9) | | |
| 5′-gtcgacattaggtgtagggagcagggttaacttccc-3′] | | |
| (SEQ ID NO: 10) | | |
| a4 (NTa4 domain) | pET32a-NTa4 | TRX-G₂-NTa4⁽¹⁻³⁹⁹⁾ |
| [mouse kidney cDNA, | | |
| 5′-gaattcggtggtatggcatctgtgtttcgaagtgaggagatg-3′ | | |
| (SEQ ID NO: 11) | | |
| 5′-gtcgactagatgatagtgtagggagctgggtttatctctcg-3′] | | |
| (SEQ ID NO: 12) | | |
| B1 (full length) | pGEX-4T1-B1(GH) | GST-G₂-B1⁽¹⁻⁵¹³⁾-G₂H₆G₂ |
| [pEF6/V5-His-TOPO-B1, | | |
| 5′-gaattcggtggtatggccacaacagtagac-3′ | | |
| (SEQ ID NO: 13) | | |
| 5′-gtcgacttaaccaccgtggtgatggtgatgatgaccgccgagcgccgtgtcggat | | |
| gcggg-3′] (SEQ ID NO: 14) | | |
| B2 (full length) | pGEX-4T1-B2(GH) | GST-G₂-B2⁽¹⁻⁵¹¹⁾-G₂H₆G₂ |
| [RAW 264.7 cDNA, | | |
| 5′-gaattcggtggtatggcgttgcgag-3′ | | |
| (SEQ ID NO: 15) | | |
| 5′-gtcgacttaaccaccgtggtgatggtgatgatgaccgccgtgttttgcagagtctc | | |
| gagggtaaaattc-3′] (SEQ ID NO: 16) | | |
| B2 (NTB2 domain) | pGEX-4T1-NTB2 | GST-G₂-B2⁽¹⁻¹¹²⁾ |
| [pAD-B2, | | |
| 5′-gaattcggtggtatggcgttgcgag-3′ | | |
| (SEQ ID NO: 17) | | |
| 5′-gtcgactaacaggatgttttcttggcgtctataccagatg-3′] | | |
| (SEQ ID NO: 18) | | |
| B2 (CTB2 domain) | pGEX-4T1-CTB2 | GST-G₂-B2⁽¹⁴⁸⁻⁵¹¹⁾ |
| [pAD-B2, | | |
| 5′-gaattcggaggagaagacttccttgacatcatgggtcag-3′ | | |
| (SEQ ID NO: 19) | | |
| 5′-gtcgacctagtgttttgcagagtctcga-3′] | | |
| (SEQ ID NO: 20) | | |

^{a}All a subunits were cloned partially, as their hydrophilic amino-terminal (NT) cytoplasmic domains; B subunits were full length; all V-ATPase subunits were of mouse origin; all PCR primers had a small, random 5′ extension (not shown) to improve restriction enzyme cleavage.
^{b}pET constructs were in pET32a(+), pGEX constructs in pGEX-4T-1; PCR products were EcoRI/SalI digested and ligated into EcoRI/SalI digested vectors; all constructs were verified by full-length insert sequencing.
^{c}Expressed protein domain organization is indicated - TRX and GST are N-terminal thioredoxin (with His-tag and S-tag domains) and glutathione-S-transferase fusions, respectively; G₂ is a Gly-Gly coupler; and G₂H₆G₂ is a C-terminal His-tag extension flanked by Gly-Gly sequences; in superscript parentheses are shown the amino acid ranges of the expressed subunits (homologous target protein subunit sequence only, numbered w.r.t. the natural subunit amino-terminal methionine).

### FULL CITATIONS FOR REFERENCES

Aguiar Bujanda, D. et al. “Assessment of Renal Toxicity and Osteonecrosis of the Jaw in Patients Receiving Zoledronic Acid for Bone Metastasis” Ann. Oncol., 2007, pp. 556-560, vol. 18.
Akesson, K. et al. “Periarticular Bone in Rheumatoid Arthritis versus Arthrosis. Histomorphometry in 103 Hip Biopsies” Acta. Orthop. Scand., 1994, pp. 135-138, vol. 65.
Anastasilakis, A. D. et al. “Oral Bisphosphonate Adverse Effects in 849 Patients with Metabolic Bone Diseases” Hormones (Athens), 2007, pp. 233-241, vol. 6.
Aota, S. et al. “Effects of Indomethacin Administration on Bone Turnover and Bone Mass in Adjuvant-Induced Arthritis in Rats” Calcif. Tissue Int., 1996, pp. 385-391, vol. 59.
Aronheim, A. et al. “Isolation of an AP-1 Repressor By A Novel Method for Detecting Protein-Protein Interactions” Mol. Cell. Biol., 1997, pp. 3094-3102, vol. 17.
Baron, R. et al. “Cell-Mediated Extracellular Acidification and Bone Resorption: Evidence for a Low pH in Resorbing Lacunae and Localization of a 100-kD Lysosomal Membrane Protein at the Osteoclast Ruffled Border” J Cell Biol, 1985, pp. 2210-2222, vol. 101.
Berlin, V. “Detecting Protein Interactions Mediated by Small Molecule Ligands” The Yeast Two-Hybrid System, 1997, pp. 259-272, Oxford University Press, New York.
Bogoch, E. et al. “Intertrochanteric Fractures of the Femur in Rheumatiod Arthritis Patients” Clin. Ortho. Rel. Res., 1993, pp. 181-186, vol. 294.
Bordier, P. et al. “Effectiveness of Parathyroid Hormone, Calcitonin and Phosphate on Bone Cells in Paget's Disease” Am. J. Med., 1974, pp. 850-857, vol. 56.
Bowman, B. J. et al. “Isolation of Genes Encoding the Neurospora Vacuolar ATPase. Analysis of vma-2 Encoding the 57-kDa Polypeptide and Comparison to vma-1” J. Biol. Chem., 1988, pp. 14002-14007, vol. 263.
Bowman, E. J. et al. “Bafilomycins: A Class of Inhibitors of Membrane ATPases from Microorganisms, Animal Cells, and Plant Cells” Proc. Natl. Acad. Sci., 1988, pp. 972-7976, vol. 85.
Brooks, J. K. et al. “Osteonecrosis of the Jaws Associated with Use of Risedronate: Report of 2 New Cases” Oral Surg. Oral Med. Oral Pathol. Oral Radiol. Endod., 2007, pp. 780-786, vol. 103.
Chen, S. H. et al. “Vacuolar H+-Atpase Binding to Microfilaments: Regulation in Response to Phosphatidylinositol 3-Kinase Activity and Detailed Characterization of the Actin-Binding Site in Subunit B” J. Biol. Chem., 2004, pp. 7988-7998, vol. 279.
Collin-Osdoby, P. et al. “RANKL-Mediated Osteoclast Formation from Murine RAW 264.7 Cells” Methods Mol. Med., 2003, pp. 153-166, vol. 80.
Day, J. S. et al. “Bisphosphonate Treatment Affects Trabecular Bone Apparent Modulus through Micro-Architecture rather than Matrix Properties” J. Orthop. Res., 2004, pp. 465-471, vol. 22.
Forgac, M. “Vacuolar ATPases: Rotary Proton Pumps in Physiology and Pathophysiology” Nat. Rev. Mol. Cell. Biol., 2007, pp. 917-929, vol. 8.
Frattini, A. et al. “Defects in TCIRG1 Subunit of the Vacuolar Proton Pump are Responsible for a Subset of Human Autosomal Recessive Osteopetrosis” Nat. Genet., 2000, pp. 343-346, vol. 25.
Gagliardi, S. et al. “5-(5,6-Dichloro-2-Indolyl)-2-Methoxy-2,4-Pentadienamides: Novel and Selective Inhibitors of the Vacuolar H+-Atpase of Osteoclasts with Bone Antiresorptive Activity” J. Med. Chem., 1998, pp. 1568-1573, vol. 41.
Garcia Saenz, J. A. et al. “Osteonecrosis of the Jaw as an Adverse Bisphosphonate Event: Three Cases of Bone Metastatic Prostate Cancer Patients Treated with Zoledronic Acid” Med. Oral Pathol Oral. Cir. Bucal., 2007, pp. E351-356, vol. 12.
Grynpas, M. D. et al. “The Effect of Rheumatoid Arthritis on Subchondral Bone” Orthopaedic Transactions, 1993, pp. 365-366, vol. 17.
Holliday, L. S. et al. “The Amino-Terminal Domain of the B Subunit of Vacuolar H+-ATPase Contains a Filamentous Actin Binding Site” J. Biol. Chem., 2000, pp. 32331-32337, vol. 275.
Johnsson, N. et al. “Split Ubiquitin—A Sensor of Protein Interactions In Vitro” Variations on the Two-Hybrid Theme, 1997, pp. 316-332, Oxford University Press, New York.
Karimova G. et al. “A Bacterial Two-Hybrid System Based on a Reconstituted Signal Transduction Pathway” Proc. Natl. Acad. Sci., 1998, pp. 5752-5756, vol. 95.
Kawasaki-Nishi, S. et al. “Yeast V-ATPase Complexes Containing Different Isoforms of the 100-kDa a-Subunit Differ in Coupling Efficiency and In Vivo Dissociation” J. Biol. Chem., 2001, pp. 17941-17948, vol. 276.
Kaye, M. et al. “Osteoclast Enlargement in Endstage Renal Disease” Kidney Int., 1985, pp. 574-581, vol. 27.
Komatsubara, S. et al. “Suppressed Bone Turnover by Long-Term Bisphosphonate Treatment Accumulates Microdamage but Maintains Intrinsic Material Properties in Cortical Bone of Dog Rib” J. Bone Miner. Res., 2004, pp. 999-1005, vol. 19.
Kominsky, S. L. et al. “A ‘Bone’ Fide Predictor of Metastasis? Predicting Breast Cancer Metastasis to Bone. J. Clin. Oncol., 2006, pp. 2227-2229, vol. 24.
Kornak, U. et al. “Mutations in the a3 Subunit of the Vacuolar H(+)-ATPase Cause Infantile Malignant Osteopetrosis” Hum. Mol. Genet., 2000, pp. 2059-2063, vol. 9.
Laitala, T. et al. “Inhibition of Bone Resorption In Vitro by Antisense RNA and DNA Molecules Targeted Against Carbonic Anhydrase II or Two Subunits of Vacuolar H(+)-ATPase”J. Clin. Invest., 1994, pp. 2311-2318, vol. 93.
Landolt-Marticorena, C. et al. “Substrate- and Inhibitor-Induced Conformational Changes in the Yeast V-ATPase Provide Evidence for Communication Between the Catalytic and Proton-Translocating Sectors” J. Biol. Chem., 1999, pp. 26057-26064, vol. 274.
Landolt-Marticorena, C. et al. “Evidence That the NH.sub.2 Terminus of Vph1p, an Integral Subunit of the V0 Sector of the Yeast V-ATPase, Interacts Directly with the vma1p and vma13p Subunits of the V1 Sector” J. Biol. Chem., 2000, pp. 15449-15457, vol. 275.
Lee, B. S. et al. “Osteoclasts Express the B2 Isoform of Vacuolar H(+)-ATPase Intracellularly and on Their Plasma Membranes” Am. J. Physiol., 1996, pp. C382-388, vol. 270.
Leng, X. H. et al. “Site-Directed Mutagenesis of the 100-kDa Subunit (vph1p) of the Yeast Vacuolar (H+)-ATPase” J. Biol. Chem., 1996, pp. 22487-22493, vol. 271. [published erratum appears in J. Biol. Chem., 1996, p. 28725, vol. 271.]
Li, Y.-P. et al. “Atp6i-Deficient Mice Exhibit Severe Osteopetrosis due to Loss of Osteoclast-Mediated Extracellular Acidification” Nature Genetics, 1999, pp. 447-451, vol. 23.
Makris, G. P. et al. “Quantitative Relationship Between Osteoclasts, Osteoclast Nuclei and the Extent of the Resorbing Surface in Hamster Periodontal Disease” Arch. Oral Biol., 1982, pp. 965-969, vol. 27.
Manolson, M. F. et al. “Identification of 3-O-(4-Benzoyl)Benzoyladenosine 5′-Triphosphate- and N,N′-Dicyclohexylcarbodiimide-Binding Subunits of a Higher Plant H+-Translocating Tonoplast ATPase” J. Biol. Chem., 1985, 12273-12279, vol. 260.
Manolson, M. F. et al. “cDNA Sequence and Homologies of the ‘57-kDa’ Nucleotide-Binding Subunit of the Vacuolar ATPase from Arabidopsis” J. Biol. Chem., 1988, pp. 17987-17994, vol. 263.
Manolson, M. F. et al. “STV1 Gene Encodes Functional Homologue of 95-kDa Yeast Vacuolar H(+)-ATPase Subunit Vph1p” J. Biol. Chem., 1994, pp. 14064-14074, vol. 269.
Manolson, M. F. et al. “The a3 Isoform of the 100-kDa V-ATPase Subunit is Highly but Differentially Expressed in Large (>or=10 Nuclei) and Small (<or=Nuclei) Osteoclasts” J. Biol. Chem., 2003, pp. 49271-49278, vol. 278.
Marx, R. E. “Pamidronate (Aredia) and Zoledronate (Zometa) Induced Avascular Necrosis of the Jaws: A Growing Epidemic” J. Oral Maxillofac. Surg., 2003, pp. 1115-1117, vol. 61.
Mashiba, T. et al. “Suppressed Bone Turnover by Bisphosphonates Increases Microdamage Accumulation and Reduces Some Biomechanical Properties in Dog Rib” J. Bone Miner. Res., 2000, 613-620, vol. 15.
Mashiba, T. et al. “Effects of Suppressed Bone Turnover by Bisphosphonates on Microdamage Accumulation and Biomechanical Properties in Clinically Relevant Skeletal Sites in Beagles” Bone, 2001a, pp. 524-531, vol. 28.
Mashiba, T. et al. “Effects of High-Dose Etidronate Treatment on Microdamage Accumulation and Biomechanical Properties in Beagle Bone before Occurrence of Spontaneous Fractures” Bone, 2001b, pp. 271-278, vol. 29.
McDermott, R. S et al. “Impact of Zoledronic Acid on Renal Function in Patients with Cancer: Clinical Significance and Development of a Predictive Model” J. Support Oncol., 2006, pp. 524-529, vol. 4.
Mundy, G. R. “Metastasis: Metastasis to Bone: Causes, Consequences and Therapeutic Opportunities” Nat. Rev. Cancer, 2002, p. 584, vol. 2.
Munier, A. et al. “Zoledronic Acid and Renal Toxicity: Data from French Adverse Effect Reporting Database” Ann. Pharmacother., 2005, pp. 1194-1197, vol. 39.
Niikura, K. et al. “A Novel Inhibitor of Vacuolar ATPase, FR167356, which Can Discriminate Between Osteoclast Vacuolar ATPase and Lysosomal Vacuolar ATPase” Br. J. Pharmacol., 2004, p. 558, vol. 142.
Niikura, K. et al. “A Novel Inhibitor of Vacuolar ATPase, FR202126, Prevents Alveolar Bone Destruction in Experimental Periodontitis in Rats” J. Toxicol. Sci., 2005, pp. 297-304, vol. 30.
Niikura, K. “Effect of a V-ATPase Inhibitor, FR202126, in Syngeneic Mouse Model of Experimental Bone Metastasis” Cancer Chemother. Pharmacol., 2007, pp. 555-562, vol. 60.
Niikura, K. et al. “FR177995, a Novel Vacuolar ATPase Inhibitor, Exerts Not Only an Inhibitory Effect on Bone Destruction but also Anti-Immunoinflammatory Effects in Adjuvant-Induced Arthritic Rats” Bone, 2007, p. 888, vol. 40.
Nishi, T. et al. “Molecular Cloning and Expression of Three Isoforms of the 100-kDa a Subunit of the Mouse Vacuolar Proton-Translocating ATPase” J. Biol. Chem., 2000, pp. 6824-6830, vol. 275.
Noji, H. et al. “Direct Observation of the Rotation of F1-ATPase”. Nature, 1997, pp. 299-302, vol. 386. [see comments]
Oka, T. et al. “a4, a Unique Kidney-Specific Isoform of Mouse Vacuolar H+-ATPase Subunit a” J. Biol. Chem., 2001, pp. 40050-40054, vol. 276.
Okahashi, N. et al. “Specific Inhibitors of Vacuolar H(+)-ATPase Trigger Apoptotic Cell Death of Osteoclasts” J. Bone Miner. Res., 1997, pp. 1116-1123, vol. 12.
Pavlakis, N. et al. “Bisphosphonates for Breast Cancer” Cochrane Database Syst. Rev., 2005, p. CD003474, vol. 3.
Roodman, G. D. “Mechanisms of Bone Metastasis” N. Engl. J. Med., 2004, pp. 1655-1664, vol. 350.
Sanna, G. et al. “Bisphosphonates and Jaw Osteonecrosis in Patients with Advanced Breast Cancer” Ann. Oncol., 2006, pp. 1512-1516, vol. 17.
Scimeca, J. C. et al. “The Gene Encoding the Mouse Homologue of the Human Osteoclast-Specific 116-kDa V-ATPase Subunit Bears a Deletion in Osteosclerotic (oc/oc) Mutants” Bone, 2000, pp. 207-213, vol. 26.
Scimeca, J. C. et al. “Novel Mutations in the TCIRG1 Gene Encoding the a3 Subunit of the Vacuolar Proton Pump in Patients Affected by Infantile Malignant Osteopetrosis” Hum. Mutat., 2003, pp. 151-157, vol. 21.
Sewell, K. et al. “Osteoporosis Therapies for Rheumatoid Arthritis Patients: Minimizing Gastrointestinal Side Effects” Semin. Arthritis Rheum., 2001, pp. 288-297, vol. 30.
Shibutani, T. et al. “Experimentally Induced Periodontitis in Beagle Dogs Causes Rapid Increases in Osteoclastic Resorption of Alveolar Bone” J. Periodontol., 1997, pp. 385-391, vol. 68.
Shimizu, S. et al. “Quantitative Histologic Studies on the Pathogenesis of Periarticular Osteoporosis in Rheumatoid Arthritis” Arthritis Rheum., 1985, pp. 25-31, vol. 28.
Smith, A. N. et al. “Molecular Cloning and Characterization of Atp6n1b: A Novel Fourth Murine Vacuolar H+-ATPase a-Subunit Gene” J. Biol. Chem., 2001, pp. 42382-42388, vol. 276.
Sobacchi, C. et al. “The Mutational Spectrum of Human Malignant Autosomal Recessive Osteopetrosis” Hum. Mol. Genet., 2001, pp. 1767-1773, vol. 10.
Sun-Wada, G. H. et al. “A Proton Pump ATPase with Testis-Specific E1-Subunit Isoform Required for Acrosome Acidification” J. Biol. Chem., 2002, pp. 18098-18105, vol. 277.
Sun-Wada, G. H. et al. “Differential Expression of a Subunit Isoforms of the Vacuolar-Type Proton Pump ATPase in Mouse Endocrine Tissues” Cell Tissue Res., 2007, pp. 239-248, vol. 329.
Sundquist, K. T., et al. “Bafilomycin A1 Inhibits Bone Resorption and Tooth Eruption In Vivo” J. Bone Miner. Res., 1994, pp. 1575-1582, vol. 9.
Susani, L. et al. “TCIRG1-Dependent Recessive Osteopetrosis: Mutation Analysis, Functional Identification of the Splicing Defects, and In Vitro Rescue by U1 snRNA” Hum. Mutat., 2004, pp. 225-235, vol. 24.
Toritsuka, Y. et al. “Osteoclastogenesis in Iliac Bone Marrow of Patients with Rheumatoid Arthritis” J. Rheumatol., 1997, pp. 1690-1696, vol. 24.
Toyomura, T. et al. “Three Subunit a Isoforms of Mouse Vacuolar H(+)-ATPase. Preferential Expression of the a3 Isoform During Osteoclast Differentiation” J. Biol. Chem., 2000, pp. 8760-8765, vol. 275.
Toyomura, T. et al. “From Lysosomes to the Plasma Membrane: Localization of Vacuolar-Type H+-ATPase with the a3 Isoform During Osteoclast Differentiation” J. Biol. Chem., 2003, pp. 22023-22030, vol. 278.
Uchida, E. et al. “Purification of Yeast Vacuolar Membrane H+-ATPase and Enzymological Discrimination of Three ATP-Driven Proton Pumps In Saccharomyces cerevisiae” Met. Enzymol., 1988, pp. 544-562, vol. 157.
Vaes, G. “On the Mechanisms of Bone Resorption. The Action of Parathyroid Hormone on the Excretion and Synthesis of Lysosomal Enzymes and on the Extracellular Release of Acid by Bone Cells” J. Cell Biol., 1968, pp. 676-697, vol. 39.
Visentin, L. et al. “A Selective Inhibitor of the Osteoclastic V-H(+)-ATPase Prevents Bone Loss in Both Thyroparathyroidectomized and Ovariectomized Rats” J. Clin. Invest., 2000, pp. 309-318, vol. 106. [see comments]
Wilkens, S. et al. “Structure of the Vacuolar ATPase by Electron Microscopy” J. Biol. Chem., 1999, pp. 31804-31810, vol. 274.
Xu, T. et al. “Subunit D (Vma8p) of the Yeast Vacuolar H+-ATPase Plays a Role in Coupling of Proton Transport and ATP Hydrolysis” J. Biol. Chem., 2000, pp. 22075-22081, vol. 275.

What is claimed is:

1. A hydrazide compound of the formula I for treating a disease or disorder requiring modulation of vacuolar (H+)-ATPases 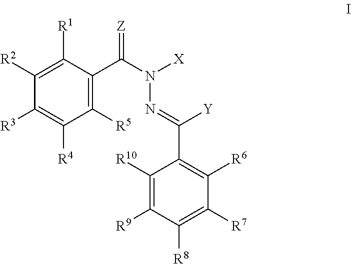
wherein
X is hydrogen or optionally substituted alkyl;
Y is hydrogen or optionally substituted alkyl, halo or alkoxy;
Z is ═O, ═S; and
R¹, R², R³, R⁴, R⁵, R⁶, R⁷, R⁸, R⁹, and R¹⁰ are independently hydrogen, optionally substituted hydroxyl, alkyl, alkenyl, alkynyl, alkylene, alkenylene, alkoxy, alkenyloxy, cycloalkyl, cycloalkenyl, cycloalkynyl, cycloalkoxy, aryl, aryloxy, arylalkoxy, aroyl, heteroaryl, heterocyclic, acyl, acyloxy, sulfonyl, sulfinyl, sulfenyl, sulfoxide, sulfate, sulfonate, amino, imino, azido, thiol, thioalkyl, thioalkoxy, thioaryl, nitro, ureido, cyano, halo, ═O, ═S, phosphonate, carboxyl, carbonyl, carbamoyl, or carboxamide, or R⁸ and R⁹ may form an aryl, cycloalkyl, heteroaryl or heterocyclic ring; or an isomer or a pharmaceutically acceptable salt thereof.

2. A hydrazide compound as claimed in claim 1, wherein the compound does not include the compounds depicted in Table 1.

3. A pharmaceutical composition comprising a hydrazide compound as claimed in claim 1 or 2, or an analog or derivative of said compound, and a pharmaceutically acceptable carrier, excipient or vehicle.

4. A method for treating and/or preventing a disorder or disease involving disruptions to normal V-ATPase activity and/or function, comprising administering a therapeutically effective amount of a compound of claim 1 or 2.

5. A method for treating and/or preventing a disorder or disease involving disruptions to normal V-ATPase activity and/or function, comprising administering a therapeutically effective amount of a composition of claim 3.

6. A method for treating and/or preventing a disorder or disease requiring modulation of V-ATPase-mediated secretion comprising administering a therapeutically effective amount of a compound of claim 1 or 2.

7. A method for treating and/or preventing a disorder or disease requiring modulation of V-ATPase-mediated secretion comprising administering a therapeutically effective amount of a composition of claim 3.

8. A method for treating and/or preventing a disorder or disease requiring regulation of intra-organellar acidification of intracellular organelles; urinary acidification; gastric acidification, bone resorption; fertility; angiogenesis; cellular invasiveness tumor cell proliferation and metastasis; and the development of drug resistance in tumor cells comprising administering a therapeutically effective amount of a compound of claim 1 or 2.

9. A method for treating and/or preventing a disorder or disease requiring regulation of intra-organellar acidification of intracellular organelles; urinary acidification; gastric acidification, bone resorption; fertility; angiogenesis; cellular invasiveness tumor cell proliferation and metastasis; and the development of drug resistance in tumor cells comprising administering a therapeutically effective amount of a composition of claim 3.

10. A method of claim 8 wherein the diseases or disorder is a bone remodeling disorder selected from the group consisting of osteoporosis, Paget's disease, achondroplasia, osteochodrytis, hyperparathyroidism, osteogenesis imperfecta, congenital hypophosphatasia, fribromatous lesions, fibrous displasia, multiple myeloma, abnormal bone turnover, osteolytic bone disease, osteomalacia, inflammatory arthritis, osteoarthritis and periodontal disease.

11. A method of claim 9 wherein the diseases or disorder is a bone remodeling disorder selected from the group consisting of osteoporosis, Paget's disease, achondroplasia, osteochodrytis, hyperparathyroidism, osteogenesis imperfecta, congenital hypophosphatasia, fribromatous lesions, fibrous displasia, multiple myeloma, abnormal bone turnover, osteolytic bone disease, osteomalacia, inflammatory arthritis, osteoarthritis and periodontal disease.

12. A method for treating and/or preventing primary osteoporosis, secondary osteoporosis, post-menopausal osteoporosis, male osteoporosissteroid induced osteoporosis, and osteoporosis caused by extended bedrest and exposure to zero gravity as experienced in space.

13. A method for increasing bone density in a mammal in need thereof comprising administering to said mammal an effective amount of a compound of claim 1 or 2.

14. A method for increasing bone density in a mammal in need thereof comprising administering to said mammal an effective amount of a composition of claim 3.

15. A method for enhancing bone formation or inhibiting bone resorption in a mammal in need thereof by administering to the mammal an effective amount of a compound of claim 1 or 2 and at least one bone enhancing agent.

16. A method for enhancing bone formation or inhibiting bone resorption in a mammal in need thereof by administering to the mammal an effective amount of a composition of claim 3 and at least one bone enhancing agent.

17. A method of claim 15 wherein the bone enhancing agent is a synthetic hormone, a natural hormone, oestrogen, calcitonin, tamoxifen, a bisphosphonate, a bisphosphonate analog, vitamin D, a vitamin D analog, a mineral supplement, a statin drug, a selective oestrogen receptor modulator and sodium fluoride.

18. A method of claim 16 wherein the bone enhancing agent is a synthetic hormone, a natural hormone, oestrogen, calcitonin, tamoxifen, a bisphosphonate, a bisphosphonate analog, vitamin D, a vitamin D analog, a mineral supplement, a statin drug, a selective oestrogen receptor modulator and sodium fluoride.

19. Use of a compound of claim 1 or 2 in the preparation of a medicament for treating and/or preventing a disease or disorder requiring modulation of V-ATPase-mediated secretion.

20. Use of a composition of claim 3 in the preparation of a medicament for treating and/or preventing a disease or disorder requiring modulation of V-ATPase-mediated secretion.

21. Use of a compound according to claim 19 wherein the disease is osteoporosis.

22. Use of a composition according to claim 20 wherein the disease is osteoporosis.

23. A kit comprising a hydrazide compound of claim 1 or 2, for preventing and/or treating a disease or disorder requiring modulation of V-ATPase-mediated secretion.

24. A kit comprising a composition of claim 3, for preventing and/or treating a disease or disorder requiring modulation of V-ATPase-mediated secretion.
